(19) United States
(12) Patent Application Publication
(10) Pub. No.: US 2006/0294612 A1
(43) Pub. Date: Dec. 28, 2006

(54) GPCR-LIKE RETINOIC ACID-INDUCED GENE 1 PROTEIN AND NUCLEIC ACID

(75) Inventors: David A. Lewin, New Haven, CT (US); Timothy A. Stewart, San Francisco, CA (US)

Correspondence Address: MERCHANT & GOULD PC, P.O. BOX 2903, MINNEAPOLIS, MN, 55402-0903, US

(73) Assignees: CuraGen Corporation, New Haven, CT (US); Genentech, Inc., South San Francisco, CA (US)

(21) Appl. No.: 11/278,477

(22) Filed: Apr. 3, 2006

Related U.S. Application Data
(62) Division of application No. 10/224,289, filed on Aug. 20, 2002, now abandoned.
(60) Provisional application No. 60/313,940, filed on Aug. 20, 2001.

(51) Int. Cl. C12Q 1/68; G01N 33/567; A01K 67/027; C07H 21/04; C12P 21/06; A61K 39/395; C07K 14/72; C07K 16/28

(52) U.S. Cl. 800/18; 435/6; 435/7.2; 435/69.1; 435/320.1; 435/325; 530/350; 530/388.22; 424/143.1; 536/23.5

(57) ABSTRACT

A novel G-protein coupled receptor-like retinoic acid induced molecule was identified as being differentially expressed (GPCR-like RAIG1) in an animal model of fasting and feeding. Compositions and methods pertaining to treatment and diagnosis of various metabolic disorders, such as cachexia and obesity.

### RELATED APPLICATIONS

This application claims priority to 60/313,940 filed Aug. 20, 2001, the entirety of which is herein incorporated by reference.

### BACKGROUND

Metabolic Disorders

Millions of people throughout the world are affected daily by metabolic disorders such as obesity, anorexia, cachexia, and diabetes. Though the causes for these disorders are as varied as the disorders themselves, many candidate genes and gene products, such as insulin, leptin, and ghrelin, have been identified as potential drug targets for treatment of these disorders.

Obesity, Anorexia, Cachexia, and Diabetes

Understanding metabolic disorders has been hampered by the absence of an animal model that immediately reflects the human situation. Human metabolic disorders do not generally follow a Mendelian inheritance pattern, wherein a single gene determines a metabolic disorder phenotype (physical manifestation of a gene's expression; Weigle and Kuijper, 1996), although there are several rodent models that do (Spiegelman and Flier, 1996; Weigle and Kuijper, 1996). Human metabolism is a quantitative trait with many genes, as well as environmental and behavioral aspects, responsible for metabolic activities and disorders (Clement et al., 1998; Montague et al., 1997; Comuzzie and Allison, 1998; Hill and Peters, 1998).

Obesity, anorexia, cachexia and diabetes are just a few examples of metabolic disorders that affect millions of people. Obesity is an excess of subcutaneous fat in proportion to lean body mass, and is related to calorie intake and use. Anorexia is a prolonged loss of appetite whereas cachexia is a general physical wasting due to malnutrition and is usually associated with chronic disease, such as certain types of cancers or infenction with the human immunodeficiency virus (HIV). Diabetes is a variable disorder of carbohydrate metabolism caused by a combination of hereditary and environmental factors and usually characterized by excessive urine production and excessive amounts of sugar in the blood and urine, as well as by thirst, hunger and loss of weight. Underlying metabolic dysfunctions contribute substantially to each of these disorders.

Fasting and Feeding

While there are many known candidate genes that may contribute to metabolic disorders (Table 1), other targets for various therapies are desirable. Optimal targets include those genes that are differentially-regulated during fasting and feeding because of their immediate relationship to food intake.

TABLE 1
SELECTED CANDIDATE GENES FOR HUMAN OBESITY/BODY
COMPOSITION (Comuzzie and Allison, 1998)

| Phenotype | Gene | Notes |
|---|---|---|
| Obesity | agouti signaling | In mutant (a^{γ}/a^{γ}) mice, agouti is |
| | polypeptide | expressed ubiquitously (instead of only |
| | | skin in wild-type mice), and antagonizes |
| | | melanocyte stimulating hormone receptor ligation. |
| | carboxypeptidase | In agouti (a^{γ}/a^{γ}) mice, a mutation in this |
| | | enzyme prevents processing of |
| | | proopiomelanocortin. |
| | leptin | In mice, encoded by ob gene; mutant |
| | | homozygotes express the obese diabetic |
| | | (db) mouse phenotype due to aberrant |
| | | leptin translational termination. |
| | leptin receptor | fa/fa (fatty) rats (Phillips et al., 1996) |
| | tubby polypeptide | May effect processing of other obesity- |
| | | related polypeptides: neuropeptide Y and |
| | | POMC (Aron et al., 1997; Guan et al., |
| | | 1998; Spiegelman and Flier, 1996; |
| | | Weigle and Kuijper, 1996). |
| | proopiomelanocortin | Knock-out mice express a phenotype |
| | (POMC) | resembling that of agouti mutants |
| | | (Yaswen et al., 1999). |
| | tumor necrosis factor-α | Genetic linkage study of Pima Indians; |
| | | upregulated in adipose tissue in obese |
| | | people and rodents (Norman et al., 1995). |
| Energy balance | uncoupling | Uncoupling proteins disengage ATP |
| | polypeptides (1, 2, 3) | synthesis from mitochondrial respiration, |
| | | thereby affecting metabolic rate |
| | | (Schrauwen et al., 1999). |
| Satiety | cholecystokinin A and | (CCK) stimulates secretion of digestive |
| | its receptor | enzyme and promotes cell growth |
| | | (Cancela, 2001). |
| Feeding | melanocortin and its | Appetite suppressants, control feeding |
| behavior | receptors (3, 4) | behavior, among many other diverse |
| | | functions (Wikberg et al., 2000). |
| Appetite | neuropeptide Y | Promote feeding, although knockout mice |
| regulation | neuropeptide Y | expressed a weaker phenotype than |
| | receptor | expected. Double mutant mice, such as |
| | | ob/ob npy-/npy- have more striking |
| | | phenotypes (Beck, 2001). |
| | ghrelin | Stimulates feeding and weight gain in |
| | | mice (Nakazato et al., 2001). |
| | other orexins | Stimulate feeding, e.g. (Sakurai et al., |
| | | 1998a; Sakurai et al., 1998b). |
| Adipocyte | peroxisome proliferator | Adipogenic transcription factor (Kersten, |
| differentiation | activated receptor-γ | 2001). |
| | β-3-adrenergic | Expressed mostly in adipocytes. May be |
| | receptor | coupled to lipolysis (Strosberg, 1997). |

Feeding behavior is dependent upon the integration of metabolic, autonomic, endocrine and environmental factors coordinated with an appropriate state of cortical arousal (wakefulness) (Willie et al., 2001). Historically, the hypothalamus has been recognized as playing a critical role in maintaining energy homeostasis by integrating environmental factors and coordinating the behavioral, autonomic, metabolic and neuroendocrine responses to these factors (Oomura, 1980; Bernardis & Bellinger 1993, 1996). Energy homeostasis is the process by which body fuel, stored in adipose tissue, is held constant over long periods of time.

Recently, researchers have greatly increased their understanding of the complex neural network that controls feeding behavior (Willie et al., 2001) (for a detailed review of peripheral and central mechanisms of feeding, see Woods et al., 1998; Elmquist et al., 1999; Kalra et al., 1999; and Salton et al., 2000). The brain controls energy homeostasis and the hormones ghrelin, leptin and insulin are crucial elements in an organism's homeostasis control system (Inui A., 2001).

Ghrelin

Ghrelin, an appetite-stimulatory peptide released from the stomach, signals to the hypothalamus when an increase in energetic demand or efficiency is encountered (Toshinai et al., 2001). Ghrelin expression is upregulated under conditions of negative energy balance and down-regulated in the setting of positive energy balance (Toshinai et al., 2001). Exogenous ghrelin administration triggers eating in rodents during the day, a time when food intake is usually nominal (Cummings et al., 2001). Based on these observations, ghrelin may act as a meal or feeding initiator.

The role ghrelin plays in energy homeostasis is best understood by examining its unique expression pattern. Ghrelin is mainly synthesized in the oxynitic glands in both the rodent and human stomach and then secreted into the systemic circulation (Tschop et al., 2001). The concentration of ghrelin peptide in stomach tissue decreases after fasting and increases after refeeding. In contrast, the plasma concentration of ghrelin increases after fasting and decreases upon refeeding. This inverse relationship seems to indicate that there is an increase in ghrelin secretion from the stomach in response to fasting, suggesting that ghrelin circulation signals the need to feed, followed by a decreased secretion upon refeeding, suggesting that satiety has been achieved (Tschop et al., 2001).

Leptin

Leptin also plays an important role in maintaining energy homeostasis. Upon cloning the leptin gene from adipocytes, researchers were able to establish that the appetite-restraining signals from adipocytes are integral components of the feedback mechanism between the peripheral nervous system and the brain in the maintenance of energy homeostasis (Toshinai et al., 2001). Leptin, encoded by the ob gene, is a circulating peptide that provides feedback information on fat stores. Gastric leptin is slightly decreased by starvation but is not significantly different in rats that have fasted for 18 hours and control animals. Upon refeeding of fasted animals, a rapid and substantial decrease was observed in gastric leptin content (Bado et al., 1998). In contrast, leptin concentration in plasma declined sharply during an 18 hour fast compared with rats fed ad libdum (Bado et al., 1998). Concomitantly, there was a threefold increase in the concentration of plasma leptin 15 minutes after the start of refeeding and a fourfold increase after 2 hours. Based on this expression pattern, a physiological role for leptin is to signal nutritional status during periods of food deprivation, thus playing a role in satiety (Inui A., 2001).

Insulin

Insulin plays an important role in maintaining energy homeostasis as a pancreatic protein that plays an essential role in the metabolism of carbohydrates and is used in the treatment and control of diabetes mellitus. Insulin helps reverse the mobilization of fuels that occurs during fasting and prepares the body for the entry of fuels from the gut.

Interactions Between Ghrelin, Leptin, and Insulin in Maintaining Energy Homeostasis

Though roles in maintaining energy homeostasis have been suggested for ghrelin, leptin and insulin, the interplay between these hormones is poorly understood. For example, although their effects on food intake are similar, leptin deficiency and insulin deficiency have opposite effects on body weight (Montague et al., 1997). Thus, while there are many candidate genes that may contribute to obesity (Table 1), therapies developed based on these genes alone are ineffective or painful. For example, leptin has entered clinical studies for treatment of obesity. In a study designed to examine the relationship between increased leptin dose and weight loss in both lean and obese adults (Heymsfield et al., 1999), only those obese subjects that received the highest dose of leptin (0.10-0.30 mg/kg/day) showed any weight loss, and some subjects actually gained weight. Furthermore, leptin administration was injected subcutaneously daily, producing enough side effects that after the first 4 weeks of the 28 week study, almost a third of the subjects declined to continue. Leptin's efficacy, when used in isolation, is at best moderate and besieged with complications. Isolated leptin administration will most likely benefit only those individuals that lack functional leptin (Farooqi et al., 1999) or suffer from other disorders, such as diabetes (Ebihara et al., 2001).

Further, it is not understood how the receptors for these hormones work in turning on or off signals such as the meal or feeding initiation signal. For example, impaired central nervous system signaling by insulin and leptin contribute to the pathogenesis of two common metabolic diseases, obesity and type II diabetes. An increased understanding into the mechanisms of regulation can lead to enhanced and effective therapies for treating metabolic disorders. The most effective therapies are likely to combine hormone products of the various genes playing a role in energy homeostasis. Therefore, optimal targets for designing effective therapies include those genes that are differentially-regulated during fasting and feeding, which signals their immediate relationship to food intake.

### SUMMARY

In a first aspect, the present invention is an isolated polypeptide having an amino acid sequence with at least 80% sequence identity to the polypeptide sequence of M. musculus GPCR-like RAIG1 (SEQ ID NO:2).

In a second aspect, the present invention is an isolated polynucleotide having a polynucleotide sequence with at least 80% sequence identity to the polynucleotide sequence of M. musculus GPCR-like RAIG1 (SEQ ID NO:1).

In a third aspect, the present invention is a method of treating metabolic disorders by modulating the activity of GPCR-like RAIG1 polypeptide.

In a fourth aspect, the present invention is a method of detecting a disorder associated with changes in GPCR-like RAIG1 gene expression by detecting a change in expression or activity of GPCR-like RAIG1 polypeptide.

In a fifth aspect, the present invention provides a method for determining whether a compound up-regulates or down-regulates the transcription of a GPCR-like RAIG1 gene by contacting the compound with a composition comprising a RNA polymerase and the gene and measuring the amount of GPCR-like RAIG1 gene transcription.

In a sixth aspect, the present invention is a transgenic non-human animal with a disrupted GPCR-like RAIG1 gene.

In a seventh aspect, the present invention is a method of screening a sample for a GPCR-like RAIG1 mutation by comparing a GPCR-like RAIG1 polynucleotide sequence in the sample with the polynucleotide sequence of GPCR-like RAIG1 (SEQ ID NO:1 or 3).

In an eighth aspect, the present invention is a method of treating a metabolic disorder by administering an antagonist or agonist to GPCR-like RAIG1.

In a ninth aspect, the present invention is directed to kits.

In a tenth aspect, the invention is a method of treating a subject with a metabolic disorder associated with dysregulated expression of GPCR-like RAIG1, said method comprising administering to the subject a substance that regulates GPCR-like RAIG1. In one embodiment, said substance is a polynucleotide or polypeptide of the invention. In another embodiment, said substance is an agonist or antagonist of the invention. In yet another embodiment, said substance is an antibody of the invention.

In an eleventh aspect, the invention provides a method for prognostic and diagnostic evaluation of a metabolic disorder and for the identificaiton of subjects exhibiting a predisposition such disorders.

In a yet another aspect, the invention provides a pharmaceutical composition for treating a metabolic disorder.

### DETAILED DESCRIPTION

GPCR-like RAIG1, a gene, has been identified that is remarkably differentially-regulated during fasting-feeding cycles, representing an important weapon in the arsenal to treat and predict treatment success in those suffering from various metabolic disorders. This gene is useful in treating metabolic disorders, as a marker for metabolic disorders for diagnosis or propensity, and as an indicator of the potential success of various treatment plans.

To identify those genes that are differentially-regulated during fasting-feeding cycles, mice were put on various feeding regimes and, at pre-determined time points, mRNA was isolated from the stomach. Expression levels in fasting and feeding mice were then assessed and compared to identify those mRNA messages that were either up- or down-regulated, using GeneCalling experiments (Shimkets et al., 1999) (see Examples) and homology searches, such as BLAST (Altschul et al., 1997), to define the encoded polypeptide. In one set of these experiments, a G-protein coupled receptor-like retinoic acid induced molecule was identified as being differentially expressed (GPCR-like RAIG1). This gene is moderately induced early in fasting, then down-regulated with extended fasting and up-regulated four-fold with feeding in recovery from fasting. The expression pattern of GPCR-like RAIG1 is shown below in Table 2.

This differentially expressed gene, its mRNA, and its polypeptide can each be manipulated in a variety of ways to treat metabolic disorders. The moderate induction of GPCR-like RAIG1 early in fasting indicates that this molecule plays a role in feeding behavior by signaling that fasting is occurring and it is time to feed. Thus GPCR-like RAIG1 is an effective appetite stimulator. Antagonists to GPCR-like RAIG1 are effective appetite suppressors. Similarly, the four-fold up-regulation of GPCR-like RAIG1 with feeding in recovery from fasting indicates that this molecule plays a role in metabolic rate, satiety, and appetite suppression, and signals for the expression and activation of molecules that play such roles. For example, if a molecule upregulated during feeding signals satiety, then increased expression of this gene, administration of the polypeptide (or its active fragments) or an agonist, to obese subjects that habitually overeat can aid the subject in diminishing the quantity of food that they need to feel satisfied. On the other hand, down regulation of this gene during fasting may represent a signal or effect on metabolic rate. For example, decreased expression of this gene, administration of the polypeptide, or an antagonist to the protein product of this gene to a subject suffering from anorexia or cachexia can aid the subject in increasing the quantity of food they need to feel satisfied.

### Embodiments

The following embodiments are given as examples of various ways to practice the invention. Many different versions will be immediately apparent to one of skill in the various arts to which this invention pertains.

Metabolic Disorder Treatment

G-Protein Coupled Receptor-like Retinoic Acid-Induced Gene 1 (GPCR-like RAIG1) can be exogenously regulated via a variety of means well-known in the art to treat or prevent metabolic disorders, including: gene therapy techniques (including cell transformation of polynucleotides encoding active portions of a gene or anti-sense oligonucleotides), small molecule antagonists and agonists, polypeptide administration (for example, in replacement therapies), antibody administration to inhibit ligand-receptor interactions, etc.

Diagnostic and Prognostic Tools

Another application of GPCR-like RAIG1 is for the prognosis and diagnosis of metabolic disorders. For example, if a subject suffering from a metabolic disorder constitutively expresses a gene that should be differentially-regulated, but is not, such as GPCR-like RAIG1, then treatments can be designed that target the expression and/or activity of that particular polypeptide. More specifically, for example, if an obese subject's expression profile (the totality of all, or preferably, a subset containing, genes known to be differentially-regulated during fasting and feeding, such as GPCR-like RAIG1) is aberrant when compared to a lean individual, then a skilled artisan can determine which genes represent therapeutic targets, thus allowing many targets to be identified simultaneously. Finally, such expression profiling can diagnose the susceptibility of a subject to become obese.

GPCR-Like RAIG1

The novel GPCR-like RAIG1 of the invention includes the polynucleotides provided in Tables 3 and 4, or fragments thereof. Mutant or variant GPCR-like RAIG1s, any of whose bases may be changed from the corresponding base shown in Tables 3 or 4 while still encoding a polypeptide that maintains the activity or physiological function of the GPCR-like RAIG1 fragment, or a fragment of such a polynucleotide, are also useful. Furthermore, polynucleotides or fragments, whose sequences are complementary to those of Tables 3 or 4 are also useful. The invention additionally includes polynucleotides or polynucleotide fragments and their complements, whose structures include chemical modifications. Such modifications include modified bases and modified or derivatized sugar phosphate backbones. These modifications are carried out at least in part to enhance the chemical stability of the modified polynucleotide such that they may be used, for example, as anti-sense binding polynucleotides in therapeutic applications. In the mutant or variant polynucleotides, and their complements, up to 20% or more of the bases may be so changed.

The invention also includes polynucleotides having 80-100% sequence identity, including 81, 82, 83, 84, 85, 86, 87, 88, 89, 90, 91, 92, 93, 94, 95, 96, 97, 98 and 99%, sequence identity to the sequences presented in Table 3, as well as polynucleotides encoding any of these polypeptides, and compliments of any of these polynucleotides. In various embodiments, polypeptides encoded by these polynucleotides have at least one, preferably all, of the native activities or physiological functions of a polypeptide comprising the polypeptide sequence encoded by the sequences presented in Table 3 or 4, and/or a polypeptide comprising the sequence presented in Table 5 or 6.

The invention also provide polypeptides having 80-100% sequence identity, including 81, 82, 83, 84, 85, 86, 87, 88, 89; 90, 91, 92, 93, 94, 95, 96, 97, 98 and 99%, sequence identity to a polypeptide encoded by the polynucleotide sequence presented in Table 3 or 4, or to the polypeptide presented in Table 5 or 6. In various embodiments, a polypeptide of the invention has at least one, preferably all, of the native activities or physiological functions of a polypeptide comprising a polypeptide sequence encoded by the sequences presented in Table 3 or 4, and/or a polypeptide comprising the sequence presented in Table 5 or 6.

The invention also provides polynucleotides that hybridize to a polynucleotide of the invention as described above. Preferably, these polynucleotides hybridize to a polynucleotide of the invention under high or moderate stringency conditions. Preferably, these polynucleotides encode polypeptides having at least one, preferably all, of the native activities or physiological functions of a polypeptide comprising the polypeptide sequence encoded by the sequences presented in Table 3 or 4, and/or a polypeptide comprising the sequence presented in Table 5 or 6.

The novel GPCR-like RAIG1 polypeptide of the invention includes the polypeptide fragments whose sequences comprise those provided in Tables 5 and 6 and fragments thereof. The invention also includes GPCR-like RAIG1 mutant or variant polypeptides, any residues of which may be changed from the corresponding residue shown in Tables 5 or 6, while still encoding a polypeptide that maintains a native activity or physiological function, or a functional fragment thereof. In the mutant or variant GPCR-like RAIG1, up to 20% or more of the residues may be so changed.

The invention further encompasses antibodies (Abs) and Ab fragments, such as F_{ab} or (F_{ab})′₂, which bind immunospecifically to any GPCR-like RAIG1 sequences of the invention.

Still further, the invention encompasses agonists and antagonists to GPCR-like RAIG1 such that ligand binding to GPCR-like RAIG1 is either enhanced or prevented, respectively.

Differentially Expressed Molecules During Fasting and Feeding

To distinguish between genes (and related polynucleotides) and the polypeptides that they encode, the abbreviations for genes are indicated by italicized (or underlined) text while abbreviations for the polypeptides are not italicized. Thus, GPCR-like RAIG1 (G-protein coupled receptor-like retinoic acid induced gene 1) or GPCR-like RAIG1 refers to the polynucleotide sequence that encodes GPCR-like RAIG1.

GPCR-Like RAIG1

In experiments examining gene expression during fasting and feeding, GPCR-like RAIG1 mRNA was found to have a complex pattern of modulation: early fasting moderately induced GPCR-like RAIG1, which was then down-regulated with extended fasting, and then up-regulated four-fold with feeding after fasting. As discussed above, this differential expression pattern clearly shows that GPCR-like RAIG1 plays an important role in metabolic signaling, such as sending a signal to feed when fasting first begins or sending a signal to stop or slow feeding once satiety has been satisfied. Table 2 sets forth the expression pattern of M. musculus GPCR-like RAIG1 during cycles of fasting and feeding.

TABLE 2
EXPRESSION LEVELS OF GPCR-LIKE RAIG1 DURING
CYCLES OF FASTING AND FEEDING

| | Observed GPCR-like |
|---|---|
| Experimental Details | RAIG1 Expression |
| 24 hr Fast v. 4 hr Fast | +2 (two-fold up-regulation of gene |
| | expression) |
| 48 hr Fast v. 24 hr Fast | −2 (two-fold down-regulation of gene |
| | expression) |
| 48 hr Fast v. 4 hr Fast | no difference observed |
| 24 hr Fast v. 24 hr Fast + 24 hr | +4 (four-fold up-regulation of gene |
| ad lib Feeding | expression) |
| 48 hr Fast v. 48 hr Fast + 24 hr | no difference observed |
| ad lib Feeding | |

Though there are literally hundreds of different types of GPCRs, the structure for the entire gene superfamily is highly conserved and serves as an important tool in recognizing the presence of a GPCR. GPCRs are integral membrane proteins typically characterized by the presence of seven hydrophobic transmembrane domains that span the plasma membrane and form a bundle of antiparallel α-helices. A hydrophobicity plot of a GPCR displays the characteristic transmembrane domains (Lewin M. J., 2001).

The transmembrane domains account for the structural and functional features of the receptor. Each of the seven transmembranes is generally composed of 20-27 amino acids. On the other hand, the N-terminal segments (7-595 amino acids), loops (5-230 amino acids), and C-terminal segments (12-359 amino acids), vary in size, an indication of their diverse structures and functions (Ji, T. H., 1998).

The N-terminus of the GPCRs is extracellular, of variable length, often glycosylated, and a common site for ligand binding, while the C-terminus is cytoplasmic and generally phosphorylated or palmitoylated leading to receptor desensitization and internalization. Extracellular loops of GPCRs alternate with intracellular loops and link the transmembrane domains. The most conserved domains of GPCRs are the transmembrane domains and the first two cytoplasmic loops. GPCRs range in size from under 400 to over 1000 amino acids (Coughlin S. R., 1994).

There are three subfamilies of GPCRs, divided based on the ligands that stimulate them and conserved key sequence motifs within phylogenetically related subfamily members. Family A (rhodopsin receptor-like, Type 1) includes small ligand-activated receptors, such as adrenaline, dopamine, peptides and glycohormones. Family B (secretin receptor-like, Type 2) consists of peptide receptors, such as calcitonin, parathyroid hormone, secretin, and vasoactive intestinal peptide. Finally family C (metabotropic glutamate receptor-like, Type 3) includes the metabotropic glutamate, calcium-sensing receptor, GABA_{B}, and pheromone receptors (see Strosberg, 1997). Type 3 (C) GPCRs characteristically contain large extracellular N-terminal domains thought to be essential in ligand and agonist binding (Galvez et al., 1999, Brauner-Osborne et al., 2000; Robbins et al., 2000). This is strikingly different from the vast majority, families A and B GPCRs, which have short N-terminal domains and agonists and ligands bind at the seventh transmembrane domain (Savarese and Fraser, 1992; Brauner-Osborne, 2000).

Recently a novel protein, retinoic acid-induced gene 1 (GPCR RAIG1) was identified by the technique of differential display (Cheng and Lotan, 1998). Expression of this gene was up-regulated by all-trans-retinoic acid (ATRA), providing further evidence that interactions may exist linking retinoic acid-mediated effects and G-protein signaling pathways (Cheng and Lotan, 1998).

Retinoids have been shown to exert cellular effect, both in vitro and in vivo on cell growth, differentiation, embryogenesis, apoptosis, and tumorigenesis; importantly they have been shown to exert significant beneficial therapeutic effects against several types of cancer (Lotan, 1996). They are believed to exert their effects by signaling through at least two nuclear receptors, the retinoic acid receptor and the retinoid X receptor. When activated the receptors bind to retinoic acid-responsive elements in the promoter regions of specific genes, resulting in activation or suppression of gene transcription (see Gudas et al., 1994: Hofmann and Eichele, 1994). Retinoids have been shown to affect directly or indirectly a multitude of genes including growth factors, interleukins, growth hormones, and extracellular matrix proteins (see Hofmann and Eichele, 1994; Gudas et al., 1994).

GPCR RAIG1 has been classified as belonging to the family of C GPCRs, the metabotropic glutamate (mGlu) receptors. Certain members of this receptor family have been shown to function in presynaptic regulatory mechanisms to control the release of neurotransmitters. In general, Gi-coupled mGlu receptor subtypes negatively modulate excitatory (and possibly also inhibitory) neurotransmitter output when activated (Schoepp D D., 2001). These receptors may have evolved to monitor glutamate that has “spilled” out of the synapse. Thus they may serve as the brain's evolutionary mechanism to prevent pathological changes in neuronal excitability and thus maintain homeostasis (Schoepp D D., 2001).

The physiological function and endogenous ligand(s) for GPCR RAIG1 and two other orphan GPCRs of type 3 (family C) remain unknown, but their induction by retinoids demonstrates that there is a link between retinoic acid and GPCR signal transduction pathways and indicates that the orphan receptors could play a role in mediating the effects of retinoic acids on embryogenesis, differentiation, and tumorigenesis (Brauner-Osborne et al., 2001).

Several molecules, such as ghrelin, leptin and the ob gene, are known to play important roles in metabolism and gut-brain signaling of satiety. Though known, human GPCR RAIG1 has never been suspected of playing a role in metabolism, especially metabolic disorders. However, the unique expression pattern of GPCR-like RAIG1 in response to the stress of fasting and feeding cycles clearly shows GPCR-like RAIG1 is another metabolic regulator, along the line of ghrelin and leptin, and itself plays important roles in metabolism and satiety signaling.

Because of its differential regulation in fasting (down-regulated) vs. feeding (up-regulated) mice, GPCR-like RAIG1 polypeptides and/or GPCR-like RAIG1-interacting polypeptides are useful as drugs or drug targets for treating metabolic diseases, including diabetes, obesity, cachexia, and anorexia. GPCR-like RAIG1 can also serve as a marker for monitoring metabolic phenomena. For example, in obese individuals (or those prone to obesity), GPCR-like RAIG1 expression or activity can be down-regulated to discourage feeding or increase metabolism. Likewise, in individuals dangerously below weight, such as those suffering from anorexia or cachexia, GPCR-like RAIG1 expression or activity can be up-regulated to promote feeding or slow metabolism.

Modulating GPCR-like RAIG1 activity in subjects suffering from cachexia is especially important, given the association of retinoids and GPCR RAIG1 expression with carcinomas. Cachexia is a wasting phenomena observed in almost half of all cancer patients, as well as individuals afflicted with other diseases, such as AIDS. In cancers, especially gastric and pancreatic, cachexia results when tumor-induced metabolic changes are disproportionate to tumor burden. Cachexia-induced weight loss leads to loss of adipose tissue and skeletal muscle mass, weakening the diaphragm and resulting in respiratory distress.

Table 3 shows the polynucleotide (mRNA) sequence of M. musculus GPCR-like RAIG1. The start codon and the stop codon are boldfaced and underlined.

TABLE 3

M. musculus GPCR-like RAIG1 polynucleotide sequence
(SEQ ID NO:1)

| | |
|---|---|
| cccacgcgtc tgcccacgcg tcgcgaccca cgcgtcctcc ttgtcccagg acctgcccag | 60 |
| tagccagggg ttgagcgctc tcgcctttca cgcgtgagtc gcaagttctc ggttcacccc | 120 |
| gagcgccgca gcgcccagga ccaaccagaa tgactactcc tacaactgcc cctagcggtt | 180 |
| gccgctcaga cctggattcc aggtaccaca gactttgtga cctggcggaa ggctggggca | 240 |
| tcgcgttgga aacactggct gcagtcgggg ctgtggctac cgtggcttgc atgtttgcac | 300 |
| tcgttttcct catctgcaaa gtgcaggact ccaacaaaag aaagatgctc cccgcccagt | 360 |
| tcctcttcct cctgggagtg ctaggggtct ttggcctcac cttcgccttc atcatcaagc | 420 |
| tggatggggc cacaggaccc acgcgcttct tcctcttcgg agtcctcttc gccatctgct | 480 |
| tctcttgcct cctggcccac gctttcaact tgatcaagct ggtccgaggg aggaagcccc | 540 |
| tgtcgtggct tgtgatcctc agtctggcgg tgggcttcag cctggtccag gacgtcattg | 600 |
| ccattgaata cctggtcctc accatgaaca ggaccaacgt ccatgtcttt tctgagctgc | 660 |
| actgctcctc ggcgcaatga ggatttcgtc catgctcctc atttacgtgc tcgtcttgat | 720 |
| ggtgctgacc ttcttcacat ccttcttggt tttctgtgga tccttctctg gctggaagag | 780 |
| acacgggttt ccacatatgc ttcacctcgt tcctctccat tgccatctgg gtggcctgga | 840 |
| tcgttctgct cctgattccc gacattgacc ggaaatggga tgataccatt ctcagcacag | 900 |
| ccttggtggc caatggctgg gttttcctgg cattttatat cttgcccgag tttcgacagc | 960 |
| tcccaaggca acggagcccc actgattacc cagttgaaga tgctttttgt aaacctcagc | 1020 |
| tcatgaagca gagctatggt gtggagaaca gagcctactc ccaagaggaa atcacccaag | 1080 |
| gtcttgagat gggggacaca ctctatgcac cttattccac ccattttcag ctacagaatc | 1140 |
| accaaaagga tttctctatc ccgagggccc aggcccggcc agtccgtaca atgactacga | 1200 |
| agggcgcaaa ggcgacacgt aagtgttggg aagagtggga caaccagagg caggtagcag | 1260 |
| gtccagccag gaatcctgct gatgtgaact gaacctcagg gcatcccggg gaaacagtac | 1320 |
| agagaggctt gcaacctgcc cagcacaccg ccgtcttgcc tggggctgct aagcctaaca | 1380 |
| aactgtcttc aaagagctcc agggtttcat ttgccccatt cctaggacac ttctgggagg | 1440 |
| tgggagtctt ggcaactccg ggtgagactc ttacctctcc cgggagtatg agcaagcctc | 1500 |
| cagtcatctg actgctcact gtttggtcat ccttggaagc cagttcacct aacccacggt | 1560 |
| gggtcctatg aggagttgct gcacacaatg caccactcaa gattcggaaa cgcccagcga | 1620 |
| agtatgcgcc ccggaagaaa cctcatcggc gtcctcggac ctttggtcca actcgccctc | 1680 |
| ccaaccggcc gccccccagg cacctggcac gaaggtcacg tgtctaccct cagtgcactg | 1740 |
| ccccacagtg gcctctcggt gcagacaccg atttccaagg gccatgtttt tatcccaatt | 1800 |
| gcctccaaac tcactgccaa ccccagaacc tctgtgtcct ttgccaggag ctctttggga | 1860 |
| cattactgga gtagacaagg tctgtttctc tctgccagga gaattgggtt tgttctcgct | 1920 |
| ataaattcct ggcc | 1934 |

Table 4 shows the polynucleotide sequence of H. sapiens GPCR-like RAIG1. The start codon and the stop codon are boldfaced and underlined.

TABLE 4

H. sapiens GPCR-like RAIG1 polynucleotide sequence
(SEQ ID NO:3)

| | |
|---|---|
| ccaaggtctc ccccagcact gaggagctcg cctgctgccc tcttgcgcgc gggaagcagc | 60 |
| accaagttca cggccaacgc cttggcacta gggtccagaa tggctacaac agtccctgat | 120 |
| ggttgccgca atggcctgaa atccaagtac tacagacttt gtgataaggc tgaagcttgg | 180 |
| ggcatcgtcc tagaaacggt ggccacagcc ggggttgtga cctcggtggc cttcatgctc | 240 |
| actctcccga tcctcgtctg caaggtgcag gactccaaca ggcgaaaaat gctgcctact | 300 |
| cagtttctct tcctcctggg tgtgttgggc atctttggcc tcaccttcgc cttcatcatc | 360 |
| ggactggacg ggagcacagg gcccacacgc ttcttcctct ttgggatcct cttttccatc | 420 |
| tgcttctcct gcctgctggc tcatgctgtc agtctgacca agctcgtccg ggggaggaag | 480 |
| cccctttccc tgttggtgat tctgggtctg gccgtgggct tcagcctagt ccaggatgtt | 540 |
| atcgctattg aatatattgt cctgaccatg aataggacca acgtcaatgt cttttctgag | 600 |
| ctttccgctc ctcgtcgcaa tgaagacttt gtcctcctgc tcacctacgt cctcttcttg | 660 |
| atggcgctga ccttcctcat gtcctccttc accttctgtg gttccttcac gggctggaag | 720 |
| agacatgggg cccacatcta cctcacgatg ctcctctcca ttgccatctg ggtggcctgg | 780 |
| atcaccctgc tcatgcttcc tgactttgac cgcaggtggg atgacaccat cctcagctcc | 840 |
| gccttggctg ccaatggctg ggtgttcctg ttggcttatg ttagtcccga gttttggctg | 900 |
| ctcacaaagc aacgaaaccc catggattat cctgttgagg atgctttctg taaacctcaa | 960 |
| ctcgtgaaga agagctatgg tgtggagaac agagcctact ctcaagagga aatcactcaa | 1020 |
| ggttttgaag agacagggga cacgctctat gccccctatt ccacacattt tcagctgcag | 1080 |
| aaccagcctc cccaaaagga attctccatc ccacgggccc acgcttggcc gagcccttac | 1140 |
| aaagactatg aagtaaagaa agagggcagc taactctgtc ctgaagagtg ggacaaatgc | 1200 |
| agccgggcgg cagatctagc gggagctcaa agggatgtgg gcgaaatctt gagtcttctg | 1260 |
| agaaaactgt acaagacact acgggaacag tttgcctccc tcccagcctc aaccacaatt | 1320 |
| cttccatgct ggggctgatg tgggctagta agactccagt tcttagaggc gctgtagtat | 1380 |
| tttttttttt ttgtctcatc ctttggatac ttcttttaag tgggagtctc aggcaactca | 1440 |
| agtttagacc cttactcttt ttgtttgttt tttgaaacag gatcttgctc tgtcacccag | 1500 |
| gcttgagtgc agtggtgcga tcacagccca gtgcagcctc gaccacctgt gctcaagcaa | 1560 |
| tcctcccatc tccatctccc aaagtgctgg gatgacaggc gtgagccaca gctcccagcc | 1620 |
| taggccctta atcttgctgt tattttccat ggactaaagg tctggtcatc tgagctcacg | 1680 |
| ctggctcaca cagctctagg ggcctgctcc tctaactcac agtgggtttt gtgaggctct | 1740 |
| gtggcccaga gcagacctgc atatctgagc aaaaatagca aaagcctctc tcagcccact | 1800 |
| ggcctgaatc tacactggaa gccaacttgc tggcaccccc gctccccaac ccttcttgcc | 1860 |
| tgggtaggag aggctaaaga tcaccctaaa tttactcatc tctctagtgc tgcctcacat | 1920 |
| tgggcctcag cagctcccca gcaccaattc acaggtcacc cctctcttct tgcactgtcc | 1980 |
| ccaaacttgc tgtcaattcc gagatctaat ctccccctac gctctgccag gaattctttc | 2040 |
| agacctcact agcacaagcc cggttgctcc ttgtcaggag aatttgtaga tcattctcac | 2100 |
| ttcaaattcc tggggctgat acttctctca tcttgcaccc caacctctgt aaatagattt | 2160 |
| accgcattta cggctgcatt ctgtaagtgg gcatggtctc ctaatggagg agtgttcatt | 2220 |
| gtataataag ttattcacct gagtatgcaa taaagatgtg gtggccactc tttcatggtg | 2280 |
| gtggcagcaa aaaaaaaaaa aa | 2302 |

Table 5 presents the M. musculus GPCR-like RAIG1 polypeptide amino acid sequence encoded by SEQ ID NO:2.

TABLE 5

M. musculus GPCR-like RAIG1 polypeptide sequence
(SEQ ID NO:2)

```
Met Thr Thr Pro Thr Thr Ala Pro Ser Gly Cys Arg Ser Asp Leu Asp
1 5 10 15
Ser Arg Tyr His Arg Leu Cys Asp Leu Ala Glu Gly Trp Gly Ile Ala
                        20 25 30
Leu Glu Thr Leu Ala Ala Val Gly Ala Val Ala Thr Val Ala Cys Met
                35 40 45
Phe Ala Leu Val Phe Leu Ile Cys Lys Val Gln Asp Ser Asn Lys Arg
        50 55 60
Lys Met Leu Pro Ala Gln Phe Leu Phe Leu Leu Gly Val Leu Gly Val
65 70 75 80
Phe Gly Leu Thr Phe Ala Phe Ile Ile Lys Leu Asp Gly Ala Thr Gly
                                85 90 95
Pro Thr Arg Phe Phe Leu Phe Gly Val Leu Phe Ala Ile Cys Phe Ser
                        100 105 110
Cys Leu Leu Ala His Ala Phe Asn Leu Ile Lys Leu Val Arg Gly Arg
                115 120 125
Lys Pro Leu Ser Trp Leu Val Ile Leu Ser Leu Ala Val Gly Phe Ser
        130 135 140
Leu Val Gln Asp Val Ile Ala Ile Glu Tyr Leu Val Leu Thr Met Asn
145 150 155 160
Arg Thr Asn Val His Val Phe Ser Glu Leu His Cys Ser Ser Ala Gln
                                165 170 175
```

Table 6 presents the H. sapiens GPCR-like RAIG1 polypeptide amino acid sequence encoded by SEQ ID NO:4. PROSITE Domain Analysis algorithm was applied to the human GPCR-like RAIG1 sequence and several sites were found. Glycosylation sites are shown in boldface. An N-glycosylation site is found at amino acids 158-161. Phosphorylation sites are shown by underlined boldface. A protein kinase C phosphorylation site is found at amino acids 59-61. Casein kinase II phosphorylation sites are found at amino acids 4-8 and 301-304. N-myristoylation sites are shown in italics underlined. N-myristoylation sites are found at amino acids 8-14, 38-43, 80-86, 88-93, 102-107, 136-142 and 201-206. Finally, amidation sites are shown in italic double underlined. An amidation site is found at amino acids 124-127.

TABLE 6

H. sapiens GPCR-like RAIG1 polypeptide sequence
(SEQ ID NO:4)

```
Met Ala Thr Thr Val Pro Asp Gly Cys Arg Asn Gly Leu Lys Ser Lys
1 5 10 15
Tyr Tyr Arg Leu Cys Asp Lys Ala Glu Ala Trp Gly Ile Val Leu Glu
                        20 25 30
Thr Val Ala Thr Ala Gly Val Val Thr Ser Val Ala Phe Met Leu Thr
                35 40 45
Leu Pro Ile Leu Val Cys Lys Val Gln Asp Ser Asn Arg Arg Lys Met
        50 55 60
Leu Pro Thr Gln Phe Leu Phe Leu Leu Gly Val Leu Gly Ile Phe Gly
65 70 75 80
Leu Thr Phe Ala Phe Ile Ile Gly Leu Asp Gly Ser Thr Gly Pro Thr
                                85 90 95
Arg Phe Phe Leu Phe Gly Ile Leu Phe Ser Ile Cys Phe Ser Cys Leu
                        100 105 110
Leu Ala His Ala Val Ser Leu Thr Lys Leu Val Arg Gly Arg Lys Pro
                115 120 125
Leu Ser Leu Leu Val Ile Leu Gly Leu Ala Val Gly Phe Ser Leu Val
        130 135 140
Gln Asp Val Ile Ala Ile Glu Tyr Ile Val Leu Thr Met Asn Arg Thr
145 150 155 160
Asn Val Asn Val Phe Ser Glu Leu Ser Ala Pro Arg Arg Asn Glu Asp
                                165 170 175
Phe Val Leu Leu Leu Thr Tyr Val Leu Phe Leu Met Ala Leu Thr Phe
                        180 185 190
Leu Met Ser Ser Phe Thr Phe Cys Gly Ser Phe Thr Gly Trp Lys Arg
                195 200 205
His Gly Ala His Ile Tyr Leu Thr Met Leu Leu Ser Ile Ala Ile Trp
        210 215 220
Val Ala Trp Ile Thr Leu Leu Met Leu Pro Asp Phe Asp Arg Arg Trp
225 230 235 240
Asp Asp Thr Ile Leu Ser Ser Ala Leu Ala Ala Asn Gly Trp Val Phe
                                245 250 255
Leu Leu Ala Tyr Val Ser Pro Glu Phe Trp Leu Leu Thr Lys Gln Arg
                        260 265 270
Asn Pro Met Asp Tyr Pro Val Glu Asp Ala Phe Cys Lys Pro Gln Leu
                275 280 285
Val Lys Lys Ser Tyr Gly Val Glu Asn Arg Ala Tyr Ser Gln Glu Glu
        290 295 300
Ile Thr Gln Gly Phe Glu Glu Thr Gly Asp Thr Leu Tyr Ala Pro Tyr
305 310 315 320
Ser Thr His Phe Gln Leu Gln Asn Gln Pro Pro Gln Lys Glu Phe Ser
                                325 330 335
Ile Pro Arg Ala His Ala Trp Pro Ser Pro Tyr Lys Asp Tyr Glu Val
                        340 345 350
Lys Lys Glu Gly Ser
                355
```

The predicted weight of M. musculus GPCR-like RAIG1, without post-translational modifications or alternative splicing, is 19206.6 Da, with a predicted pI of 8.34. Table 7 presents other predicted physical characteristic of the GPCR-like RAIG1 polypeptide (SEQ ID NO:2).

TABLE 7
Predicted physical properties of mouse GPCR-like RAIG1

| | | | | |
|---|---|---|---|---|
| 276 nm | 278 nm | 279 nm | 280 nm | 282 nm |

Values assuming ALL Cys residues appear as half cystines:

| | | | | | |
|---|---|---|---|---|---|
| Extinction | 62485 | 63381 | 63125 | 62520 | 60700 |
| Coefficient | | | | | |
| Optical | 1.552 | 1.575 | 1.568 | 1.553 | 1.508 |
| Density | | | | | |

Values assuming NO Cys residues appear as half cystines:

| | | | | | |
|---|---|---|---|---|---|
| Extinction | 62050 | 63000 | 62765 | 62160 | 60400 |
| Coefficient | | | | | |
| Optical | 1.542 | 1.565 | 1.559 | 1.544 | 1.501 |
| Density | | | | | |

Note:
The conditions at which these equations are valid are: pH 6.5, 6.0 M guanidium hydrochloride, 0.02 M phosphate buffer.

GPCR RAIG1 (SEQ ID NO:3), the human homolog of M. musculus GPCR-like RAIG1, is a 357 amino acid polypeptide with a predicted molecular weight of 40250.6 Da and a predicted pI of 8.12. Other predicted and observed physical characteristics of GPCR RAIG1 are set forth in Table 8.

TABLE 8
Predicted physical properties of human GPCR RAIG1

| | | | | |
|---|---|---|---|---|
| 276 nm | 278 nm | 279 nm | 280 nm | 282 nm |

Values assuming ALL Cys residues appear as half cystines:

| | | | | | |
|---|---|---|---|---|---|
| Extinction | 62485 | 63381 | 63125 | 62520 | 60700 |
| Coefficient | | | | | |
| Optical | 1.552 | 1.575 | 1.568 | 1.553 | 1.508 |
| Density | | | | | |

Values assuming NO Cys residues appear as half cystines:

| | | | | | |
|---|---|---|---|---|---|
| Extinction | 62050 | 63000 | 62765 | 62160 | 60400 |
| Coefficient | | | | | |
| Optical | 1.542 | 1.565 | 1.559 | 1.544 | 1.501 |
| Density | | | | | |

Note:
The conditions at which these equations are valid are: pH 6.5, 6.0 M guanidium hydrochloride, 0.02 M phosphate buffer.

Homology to other molecules was found using BLASTX (Altschul et al., 1990) and CLUSTALW software for nearest neighbors (Thompson et al., 1994). The following sequences were compared for homology using the CLUSTALW software: the novel M. musculus GPCR-like RAIG1 (SEQ ID NO:1, designated as “pg_mm_gbh_af095448_h0t0426.4_E”), human RECAP (SEQ ID NO:5, AX078375.1HsSeq43 PatWO0107612), human GPCR-like RAIG1 (SEQ ID NO:3, AF095448.1HsPutativeGPCR_GPCR RAIG1), mouse orphan GPRC5D (SEQ ID NO:6, AF218809MnGPRC5D), human orphan GPRC5C (SEQ ID NO:7, AF207989_HsGPRC5C), human orphan GPRC5D (SEQ ID NO:8, AF209923_HsGPRC5D), and mouse GPCR RAI protein 3 gene (SEQ ID NO:9, AF376131MmGPCRRAIProt3gene). Higly conserved regions (black) indicate those regions of the polypeptide that are most important for function. The results of the CLUSTALW alignment are seen in Table 9.

TABLE 9
ClustalW DNA Sequence Alignment Analysis

Sequence type explicitly set to DNA
Sequence format is Pearson
Sequence 1: AX078375.1HsSeq43PatWO0107612 1619 bp (SEQ ID NO:5)
Sequence 2: AF095448.1HsPutativeGPCR_GPCR RAIG1 2302 bp (SEQ ID NO:3)
Sequence 3: pg_mm_gbh_af095448_h0t0426.4_E 1934 bp (SEQ ID NO:1)
Sequence 4: AF218809MmGPRC5D 1324 bp (SEQ ID NO:6)
Sequence 5: AF207989_HsGPRC5C 1864 bp (SEQ ID NO:7)
Sequence 6: AF209923_HsGPRC5D 1078 bp (SEQ ID NO:8)
Sequence 7: AF376131MmGPCRRAIProt3gene 3764 bp (SEQ ID NO:9)
Mulitple Alignment:
AX078375.1HsSeq43PatWO0107612 **** ------------------------------------------------------------ **
AF095448.1HsPutativeGPCR_RAIG1 **** ------------------------------------------------------------ **
pg_mm_gbh_.af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPRCAIProt3gene 1 GGNAANTNNTNCTTNNTNCACNNNTCNNTNGNNNTNNGNANNNCCTCCTCCTCCTCCTCC
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene ∠ 61 TCCTCCTCCTCCTCCTCCTCCTCCTTCTTCTTCTTCTTCTTCTTCTTCTTCTTCTTCTTC 1
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 121 GATTTACTGATTTACTTTGTAAGTACACTGAAGCTGTCTTCAGACACACCAGACGAGGCA 1
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t04626.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 181 TCCTATCCCATTAGAGATGGTTGTGAGTCACCACGTGGTTGCTGGGATTTGAACTCAGGA 2
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 241 TATTTAGAAGAACAGTCAGTGCTCTTAACCTCTGAGCCATCTCTCCAGTCCATCCATGGT 3
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 301 TCTCTTGGATACTGGTCTAGCTTCAGTTTCTATAGATGAGATTGTAGAAGTTTAGAGAGA 3
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 361 AGCCCTTTCCAGCCTCATCATTCCCCAAGTCCTTTCCCCAGGCCCCTGTCTCTTGGTGAA 4
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 421 CCCCAGGAGAGGAGGAGAACTTGTCGACCTCTGACCTGCAGGCCACAAGGGAGATGGCCA 4
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 481 AGTTAAATATTGACTGCGGTTTCTTAGGTACGAGATCAATAGAAATATAGACTCCAAAGA 5
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AG376131MmGPCRRAIProt3gene 541 TTTAGGTTGTTTTGGCTCTGGTGTTTGAACCAGTACCTTATACATCACAGCATAAGCTCA 6
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AG376131MmGPCRRAIProt3gene 601 TGAAGAGTCTTTCTATTGGTGTGTGTGTGTGTGTGTGTGTGTGTGTGTGTGTGTGTGTGT 6
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AG376131MmGPCRRAIProt3gene 661 GTGTGTGTATGTGCGCGTGTGTGTAGCACTCACCATGGTCTTGTGTATAGGTCAGAGGAC 7
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AG376131MmGPCRRAIProt3gene 721 AACTTTACAGTCTTGGTTCTCTCCTTGGACTTGGTTGACCTTCGTGCCGATTTTGGGAAT 7
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 781 TGGACTCTGGTTAGGCAGCACGTGGTTTATTGGCTGACCTTCCCATCTCTCTGCCTTTTT 8
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 841 GAGTCAGGCTCTTGAGAGTAGCCCCCCCCTCCCCCTTTCCCGTTGCCCTATATCACTATG 9
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 901 TGGCTCAGGCTGGTGGCTATCTTCCTGTTTGAGTCTCCCAAATGCTGACTCGAACTGTCA 9
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 961 TTTGCAGCAAGTCACGTTCAGGTGACCTCAAACTCCTATGTAGTAGAAACTGGCCTTGGA 10
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 1021 GTGTTTTTCTACCTGCTTCTACCACCCAAGTGCTGGCATTACAGAGGTGCCCCGCGCCAG 10
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 1081 GCCTGGCTTGTTACATTTTATTCTTTTCTTTTCTTTTTTTGAGACAGGGTTTCTCTGTGC 11
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 1141 ATCCCGGATTGTTCTGGAACTCACTGTGTAGAGCAGACTGACCTCCAATTCACTGAGATC 12
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 1201 TGCTTGCCTCTCCCTTAGGAGTGCTGGGATTAAAGTTGTGAGCCCCTACCATCTGGCAAG 12
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AG376131MmGPCRRAIProt3gene 1261 ATTGTTTGTTACATTTTAAAAAGCATTTAAGGGTGTGGAGAAAAGTGGGTCTCAGCCCCT 13
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 1321 TGTAGGGGTTAAACATCTTTTTCACAGGGGTCACCTAAGCCCAGTAAAAAGCACAGATAT 13
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E **** ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AG376131MmGPCRRAIProt3gene 1381 GTATATTAAGATTCATAACAGAAGCAAAATTACAGTTATGAGGTAATAACAAAGATAATT 14
AX078375.1HsSeq43PatWO0107612 **** ------------------------------------------------------------ **
AF095448.1HsPutativeGPCR_RAIG1 **** ------------------------------------------------------------ **
pg_mm_gbh_af095448_h0t0426.4_E 1 ----------------------------------------------CCCACGCGTCTGCC
AF218809MmGPRC5D **** ------------------------------------------------------------ **
AF209923_HsGPRC5D **** ------------------------------------------------------------ **
AF207989_HsGPRC5C 1 --------------------------GGNAANNNANNNNANGNTNCTNNCTNGNCCNNNA
AF376131MmGPCRRAIProt3gene 1441 TTATGGCTGGGGGTCGCCATACATGAAAAATTGTATTAAAACTGAACTAAAGCACTTAGA 15

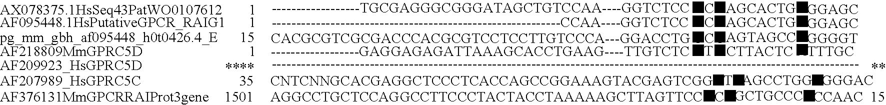

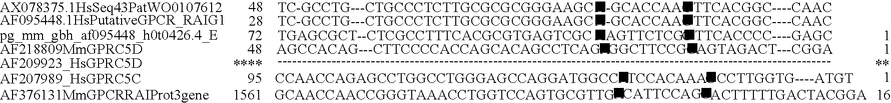

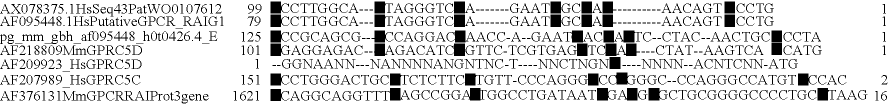

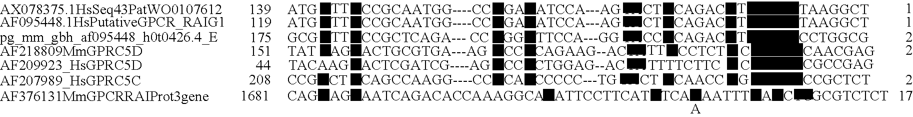

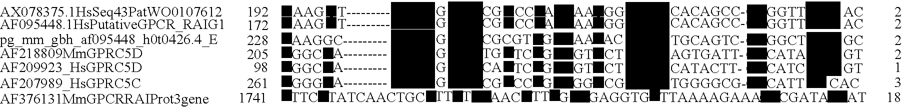

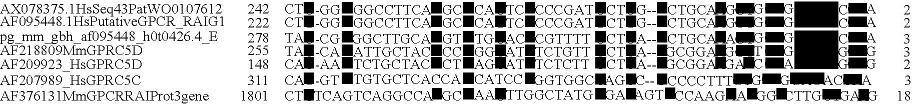

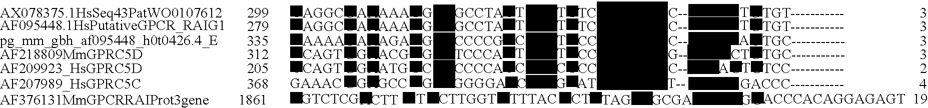

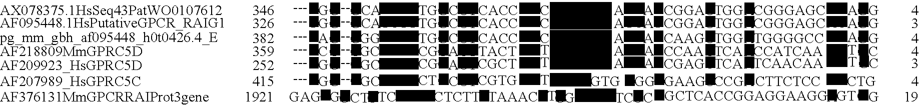

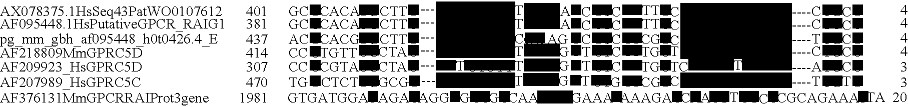

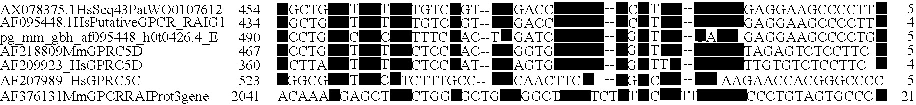

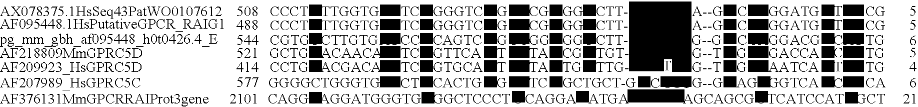

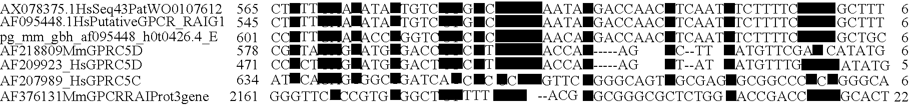

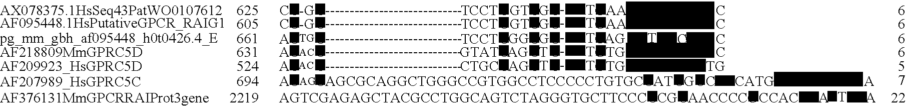

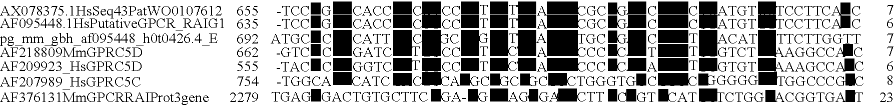

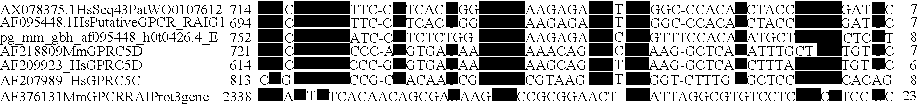

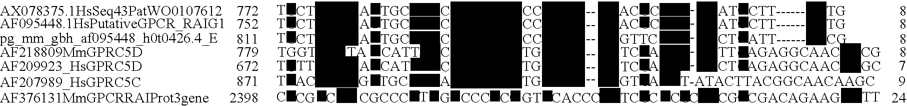

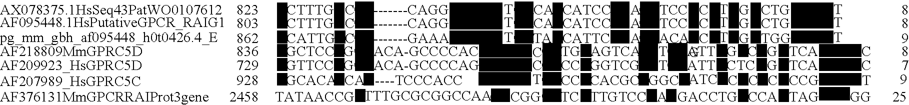

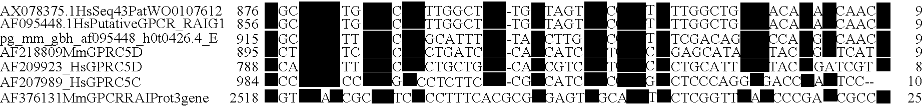

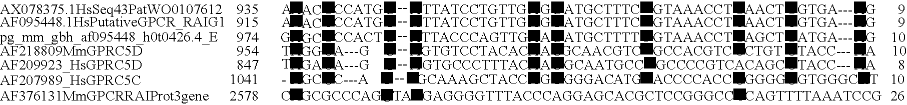

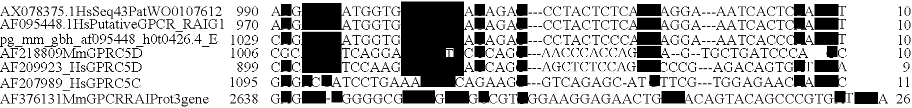

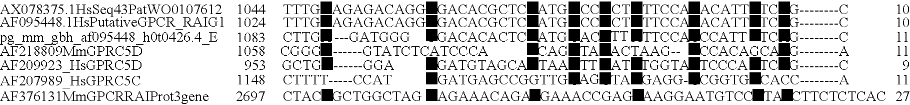

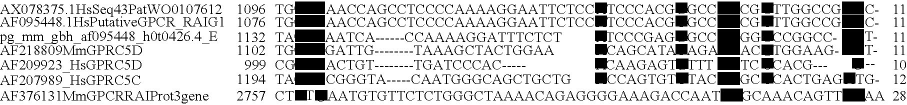

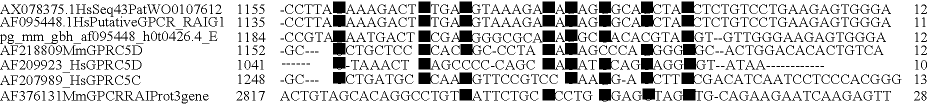

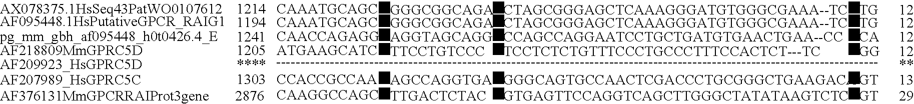

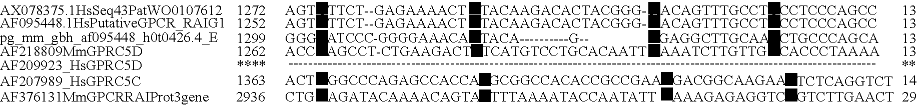

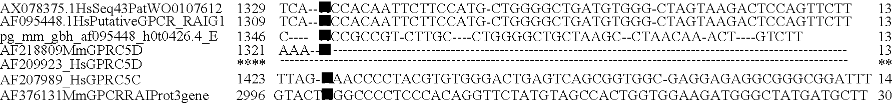

AX078375.1HsSEQ43PatWO0107612 1385 AGAGGCGCTGTAGTATTTTTTTTTTTTTTTGGCTCATCCTTAGGATACTTCTTTTAAGTG 14
AF095448.1HsPutativeGPCR_RAIG1 1365 AGAGGCGCTGTAGTATTTTTTTTTTTTTGT--CTCATCCTTTGGATACTTCTTTTAAGTG 14
pg_mm_gbh_af095448_h0t0426.4_E 1390 CAAAGAGCTCCAGGGTTTCATTT-----GC--CCCATTCCTAGGACACTTCTGGGAGGTG 14
AF218809MmGPRC5D **** ------------------------------------------------------------ **
AF209923_HsGPRC5D **** ------------------------------------------------------------ **
AF207989_HsGPTC5C 1481 GGGGAGGGCCCTGAGGACCTGGCCCCGGGCAAGGGACTCTCCAGGCTCCTCCTCCCCCTG 15
AF376131MmGPCRRAIProt3gene 3056 TCAACTCCCATCTGAGTTTCAAGGGTCTGTGAATGAATATAGGGGTAGAGGGAGAGGATG 31
AX078375.1HsSeq43PatWO0107612 1445 GGAGTCTCAGGCAACTCAAGTTTAGACCCTTACTCTTTTTGTTTGTTTTTTGAAAC---- 13
AF095448.1HsPutativeGPCR_RAIG1 1423 GGAGTCTCAGGCAACTCAAGTTTAGACCCTTACTCTTTTTGTTTGTTTTTTGAAAC---- 14
pg_mm_gbh₊₁₁₃ af095448_h0t0426.4_E 1443 GGAGTCTT-GGCAACTCCGGGTGAGACTCTTACCTCTCCCGGGAGTATGAGCAAGCCTCC 15
AF218809MmGPRC5D **** ------------------------------------------------------------ **
AF209923_HsGPRC5D **** ------------------------------------------------------------ **
AG207989_HsGPRC5C 1541 CAGGCCA--GCAACATGTGCCCCAGATCTGGAAGGGCCTCCCTCTCTGCCAGTGTTTGG 15
AF376131MmGPCRRAIProt3gene 3116 CCAGCTGCTTCCTTCCCAAAGGAGCGAAGGCCTTGGAGGTCAGGAAGTGACTCACCTCAA 31
AX078375.1HsSeq43PatWO0107612 15011 AGG-ATCT---TGCTC--TGTCACC-CAGGCTTGAGTGC-AGTGGTGCGATCACAGCCCA 11
AF095448.1HsPutativeGPCR_RAIG1 1479 AGG-ATCT---TGCTC--TGTCACC-CAGGCTTGAGTGC-AGTGGTGCGATCACAGCCCA 12
pg_mm_gbh_af095448_h0t0426.4_E 1502 AGTCATCTGACTGCTGACTGTTTGGTCATCCTTGGAAGCCAGTTCACCTAACCCA---CG 13
AF218809MmGPRC5D **** ------------------------------------------------------------ **
AF209923_HsGPRC5D **** ------------------------------------------------------------ **
AF207989_HsGPRC5C **** GTGGGTGTCATGGGTG--TCCCCACCCACTCCTCAGTGTTTGTGGAGTCGAGGAG-CCA 14
AF376131MmGPCRRAIProt3gene 3176 CGCCCCCCCCCTTCTCATGTGCGTGTTGTTCCTCCACCCCATCCCTTACTTTTCCTGTTG 32
AX078375.1HsSeq43PatWO0107612 1553 GTGCAGCCTC-GACCACCTG-TGCTCAAGCAATCCTCCCATCTCCATCTCCCAAAGTGCT 14
AF095448.1HsPutativeGPCR_RAIG1 1531 GTGCAGCCTC-GACCACCTG-TGCTCAAGCAATCCTCCCATCTCCATCTCCCAAAGTGCT 15
pg_mm_gbh_af095448_h0t0426.4_E 1559 GTGGGTCCTATGAGGAGTTGCTGCACA--CAATGCACCACTCAAGATTCGGAAACGCCCA 16
AF218809MmGPRC5D **** ------------------------------------------------------------ **
AF209923_HsGPRC5D **** ------------------------------------------------------------ **
AF207989_HsGPRC5C 1656 CCCCAGCCTCCTGCCAGGATCACCTCGGCGGTCACACTCCAGCCAAATAGTGTTCTCGGG 17
AF376131MmGPCRRAIProt3gene 3236 CCCCAGTCTCTAAGACCCTGTCTCTCTTTAACAAGAGGGGGTGAAAAGCTTGTCTTCCCG 32
AX078375.1HsSeq43PatWO0107612 1611 GGGATGACA--------------------------------------------------- 14
AF095448.1HsPutativeGPCR_RAIG1 1589 GGGATGACAGGCGTGAGCCACAGCTCCCAGCCTAGGCCCT--TAATCTTGCTGTTATTTT 15
pg_mm_gbh_af095448_h0t0426.4_E 1617 GCGAAGTATGCGCCCCGGAAGAAACCTCATCGGCGTCCTC--GGACCTTTGGTCCAACTC 16
AF218809MmGPRC5D **** ------------------------------------------------------------ **
AF209923_HsGPRC5D **** ------------------------------------------------------------ **
AF207989_HsGPRC5C 1716 GTGGTGGCTGGGCAGCGCCTATGTTTCTCTGGAGATTCCT--GCAACCTCAAGAGACTTC 17
AF376131MmGPCRRAIProt3gene 3296 GAGGAGAAGAGACACAGGCAGGAGGAGGCTGGCAGATCACAATGAATGGAAAGACAGGAA 33
AX078375.1HsSeq43PatWO0107612 **** ------------------------------------------------------------ **
AF095448.1HsPutativeGPCR_RAIG1 1647 CCATGGACTAAAGGTCTGGTCATCTGAGCTCACGCTGGCTCACACAGCTCTAGGGGCCTG 17
pg_mm_gbh_af095448_h0t0426.4_E 1675 GCCCTCCCAACCGGCC--GCCCCCCAGGCACCTGGCACGAAGGTCACGTGTCTACCCTCA 17
AF218809MmGPRC5D **** ------------------------------------------------------------ **
AF209923_HsGPRC5D **** ------------------------------------------------------------ **
AF207989_HsGPRC5C 1774 CCAGGCGCTCAGGCCT--GGATCTTGCTCCTCTGTGAGGAACAAGGGTGCCTAATAAATA 18
AF376131MmGPCRRAIProt3gene 3356 ACAGCAGTTGTTTTTTTTTTTTTATTGTTGTTGTTTTTTTGTTTGTTTGTTTGTTTTAGA 34
AX078375.1HsSeq43PatWO0107612 **** ------------------------------------------------------------ **
AF095448.1HsPutativeGPCR_RAIG1 1707 CTCCTCTAACTCACAGTGGGTTTTGTGAGGCTCTGTGGCCCAGAGCAGACCTGCATATCT 17
pg_mm_gbh_af095448_h0t0426.4_E 1733 GTGCACTGCCCCACAGTGGCCTCTCGGTGCAGACACCGATTTCCAAGGGCCATGTTTTTA 17
AF218809MmGPRC5D **** ------------------------------------------------------------ **
AF209923_HsGPRC5D **** ------------------------------------------------------------ **
AF207989_HsGPRC5C 1832 CATTTCTGCTTTATTAAAAAAAAAAAAAAAAAA--------------------------- 18
AF376131MmGPCRRAIProt3gene 3416 CATTCAGGCTGGCCTCGAGCTTGTAACCACCATTCTGCCTCAGTTTGCAGAGTGTTGGGA 34
AX078375.1HsSeq43PatWO0107612 **** ------------------------------------------------------------ **
AF095448.1HsPutativeGPCR_RAIG1 1767 GAGCAAAAATAGCAAAAGCCTCTCTCAGCCCACTGGCCTGAATCTACACTGGAAGCCAA- 18
pg_mm_gbh_af095448_h0t0426.4_E 1793 TCCCATTGCCTCCAAACTCACTGCCAACCCCAGAACCTCTGTGTCCTTTGCCAGGAGCT 18
AF218809MmGPRC5D ∠ **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 3476 TAACAGGTATCGGTCTCATGGTAGAAGGCTCCCAAATGGCAAAAGCCTGCCTGGAAGCAG 35
AX078375.1HsSeq43PatWO0107612 **** ------------------------------------------------------------ **
AF095448.1HsPutativeGPCR_RAIG1 1826 CTTGCTGGCACCCCCGC--TCCCCAACCCTTCTTGCCTGGGTAGGAGAGGCTAAAGATCA 18
pg_mm_gbh_af095448_h0t0426.4_E 1853 CTTTGGGACATTACTGGAGTAGACAAGGTCTGTTTCTCTCTGCCAGGAGAATTGGGTTTG 19
AF218809MmGPRC5D ∠ **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 3536 GTAATTGAAGGACCATCACCGTGCCTGCCTGCCTTTTCTCCAGGCAAGTGTCTGAGGCTT 35
AX078375.1HsSeq43PatWO0107612 **** ------------------------------------------------------------ **
AF095448.1HsPutativeGPCR_RAIG1 1884 CCCTAAATTTACTCTCTCTCTAGTGCTGCCTCACATTGGGCCTCAGCAGCTCCCCAGCA 15
pg_mm_gbh_af095448_h0t0426.4_E 1913 TTCTCGCTATAAATTCCTGGCC-------------------------------------- 15
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 3596 TTGTTGAAGCCTGGTGAAAGGTTCTCTGGAAACAGTCTCTTCTTTGCTCTTTACTGAAGG 36
AX078375.1HsSeq43PatWO0107612 **** ------------------------------------------------------------ **
AF095448.1HsPutativeGPCR_RAIG1 1944 CCAATTCACAGGTCACCCCTCTCTTCTTGCACTGTCCCCAAACTTGCTGTCAATTCCGAG 26
pg_mm_gbh_af095448_h0t0426.4_E 1934 ------------------------------------------------------------ 19
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene 3656 AGAATTCTTTAGCCACTTCAGATACAATGGCCTGGGAAGGTGGAGGGGAGGGTGAGGCCA 36
AX078375.1HsSeq43PatWO0107612 **** ------------------------------------------------------------ **
AF095448.1HsPutativeGPCR_RAIG1 2004 ATCTAATCTCCCCCTACGCTCTGCCAGGAATTCTTTCAGACCTCACTAGCACAAGCCCGG 28
pg_mm_gbh_af095448_h0t0426.4_E 1934 ------------------------------------------------------------ 15
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIPro3gene 3716 ACACTTCACCCTTAGGGGTTTTTGTACTGGCCATCACTGATAGAGAGAA------------ 37
AX078375.1HsSeq43PatWO0107612 **** ------------------------------------------------------------ **
AF095448.1HsPutativeGPCR_RAIG1 2064 TTGCTCCTTGTCAGGAGAATTTGTAGATCATTCTCACTTCAAATTCCTGGGGCTGATACT 22
pg_mm_gbh_af095448_h0t0426.4_E 1934 ------------------------------------------------------------ 15
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene **** ------------------------------------------------------------ **
AX078375.1HsSeq43PatWO0107612 **** ------------------------------------------------------------ **
AF095448.1HsPutativeGPCR_RAIG1 2124 TCTCTCATCTTGCACCCCAACCTCTGTAAATAGATTTACCGCATTTACGGCTGCATTCTG 21
pg_num_gbh_af095448_h0t0426.4_E 1934 ------------------------------------------------------------ 15
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPRCRRAIProt3gene **** ------------------------------------------------------------ **
AX078375.1HsSeq43PatWO0107612 **** ------------------------------------------------------------ **
AF095448.1HsPutativeGPCR_RAIG1 2184 TAAGTGGGCATGGTCTCCTAATGGAGGAGTGTTCATTGTATAATAAGTTATTCACCTGAG 21
pg_mm_gbh_af095448_h0t0426.4_E 1934 ------------------------------------------------------------ **
AF218809MmGPRC5D **** ------------------------------------------------------------ **
AF209923_HsGPRC5D **** ------------------------------------------------------------ **
AF207989_HsGPRC5C **** ------------------------------------------------------------ **
AF376131MmGPCRRAIProt3gene **** ------------------------------------------------------------ **
AX078375.1HsSeq43PatWO0107612 **** ------------------------------------------------------------ **
AF095448.1HsPutativeGPCR_RAIG1 2244 TATGCAATAAAGATGTGGTGGCCACTCTTTCATGGTGGTGGCAGCAAAAAAAAAAAAAA 21
pg_num_gbh_af095448_h0t0426.4_E 1934 ------------------------------------------------------------ 15
AF218809MmGPRC5D **** ∩------------------------------------------------------------ **
AF209923_HsGPRC5D **** ------------------------------------------------------------ **
AF207989_HsGPRC5C **** ------------------------------------------------------------ **
AF376131MmGPCRRAIProt3gene **** ------------------------------------------------------------ **

BLASTX was used to compare novel mouse GPCR-like RAIG1 (SEQ ID NO:2) and human GPCR RAI3 (SEQ ID NO:10). The results of the BLASTX comparison are seen in Table 10.

TABLE 10
BLASTX comparison of mouse GPCR-like RAIG1 and human RAI3

Query Sequence:
Novel mouse contig described in this disclosure:
pg_mm_gbh_af095448_h0t0426.4_EXT
Subject Sequence:
ptnr:SPTREMBL-ACC:O95357 PUTATIVE G PROTEIN-COUPLED RECEPTOR (CDNA FLJ10899 FIS,
CLONE NT2RP5003506) (RETINOIC ACID INDUCED 3) - Homo sapiens (Human), 357 aa.
GenBank ACC:
Plus Strand HSPs:
Score = 1168 (411.2 bits), Expect = 6.5e − 121, Sum P(2) = 6.5e − 121
Identities = 237/342 (69%), Positives = 266/342 (77%), Frame = +3

| | | | |
|---|---|---|---|
| Query: | 162 | TTAPSGCRSDLDSRYHRLCDLAEGWGIALETLAAVGAVATVACMFALVFLICKVQDSNKR | 341 |
| | | TT P GCR+ L S+Y+RLCD AE WGI LET+A G V +VA M L L+CKVQDSN+R | |
| Sbjct: | 3 | TTVPDGCRNGLKSKYYRLCDKAEAWGIVLETVATAGVVTSVAFMLTLPILVCKVQDSNRR | 62 |
| Query: | 342 | KMLPAQFLFLLGVLGVFGLTFAFIIKLDGATGPTRFFLFGVLFAICFSCLLAHAFNLIKL | 521 |
| | | KMLP QFLFLLGVLG+FGLTFAFII LDG+TGPTRFFLFG+LF+ICFSCLLAHA +L KL | |
| Sbjct: | 63 | KMLPTQFLFLLGVLGIFGLTFAFIIGLDGSTGPTRFFLFGILFSICFSCLLAHAVSLTKL | 122 |
| Query: | 522 | VRGRKPLSWLVILSLAVGFSLVQDVIAIEYLVLTMNRTNVHVFSELHCSSAQ*GFRPCSS | 701 |
| | | VRGRKPLS LVIL LAVGFSLVQDVIAIEY+VLTMNRTNV+VFSEL F + | |
| Sbjct: | 123 | VRGRKPLSLLVILGLAVGFSLVQDVIAIEYIVLTMNRTNVNVFSELSAPRRNEDFVLLLT | 182 |
| Query: | 702 | FTCSS*WC*PSSHPSWFSVDPSLAG-RDTGFHICFTSFLSIAIWVAWIVLLLIPDIDRKW | 878 |
| | | + + S F+ S G + G HI T LSIAIWVAWI LL++PD DR+W | |
| Sbjct: | 183 | YVLFLMAL--TFLMSSFTFCGSFTGWKRHGAHIYLTMLLSIAIWVAWITLLMLPDFDRRW | 240 |
| Query: | 879 | DDTILSTALVANGWVFLAFYILPEFRQLPRQRSPTDYPVEDAFCKPQLMKQSYGVENRAY | 1058 |
| | | DDTILS+AL ANGWVFL Y+ PEF L +QR+P DYPVEDAFCKPQL+K+SYGVENRAY | |
| Sbjct: | 241 | DDTILSSALAANGWVFLLAYVSPEFWLLTKQRNPMDYPVEDAFCKPQLVKKSYGVENRAY | 300 |
| Query: | 1059 | SQEEITQGLE-MGDTLYAPYSTHFQLQNH--QKDFSIPRAQARP | 1181 |
| | | SQEEITQG E GDTLYAPYSTHFQLQN QK+FSIPRA A P | |
| Sbjct: | 301 | SQEEITQGFEETGDTLYAPYSTHFQLQNQPPQKEFSIPRAHAWP | 344 |

Score = 121 (42.6 bits), Expect = 1.5e − 05, Sum P(3) = 1.5e − 05
Identities = 22/32 (68%), Positives = 24/32 (75%), Frame = +2

| | | | |
|---|---|---|---|
| Query: | 692 | MLLIYVLVLMVLTFFTSFLVFCGSFSGWKRHG | 787 |
| | | +LL YVL LM LTF S FCGSF+GWKRHG | |
| Sbjct: | 179 | LLLTYVLFLMALTFLMSSFTFCGSFTGWKRHG | 210 |

Score = 52 (18.3 bits), Expect = 6.5e − 121, Sum P(2) = 6.5e − 121
Identities = 15/40 (37%), Positives = 19/40 (47%), Frame = +2

| | | | |
|---|---|---|---|
| Query: | 1109 | TLFHPFSA-----TESP-KGFLYPEGPGPASPYNDYEGRK | 1210 |
| | | TL+ P+S + P K F P SPY DYE +K | |
| Sbjct: | 315 | TLYAPYSTHFQLQNQPPQKEFSIPRAHAWPSPYKDYEVKK | 354 |

Score = 52 (18.3 bits), Expect = 1.5e − 05, Sum P(3) = 1.5e − 05
Identities = 10/14 (71%), Positives = 11/14 (78%), Frame = +1

| | | | |
|---|---|---|---|
| Query: | 649 | FLSCTAPRRNEDFV | 690 |
| | | F +APRRNEDFV | |
| Sbjct.: | 165 | FSELSAPRRNEDFV | 178 |

NOTE:
M. musculus GPCR-like RAIG1 (mouse contig pg_mm_gbh_af095448_h0t0426.4_EXT) is partial polynucleotide sequence representing the mouse homolog of the human GPCR, RAI3 (GenBank AF095448, SEQ ID NO:3). There is no evidence that this protein has previously been associated with metabolic disorders.

BESTFIT PROTEIN SEQUENCE ALIGNMENT analysis was carried out between human GPCR RAIG1 (SEQ ID NO:4, designated as “AF095448.1”) and mouse GPCR-like RAIG1 (SEQ ID NO:2, designated as “pg_mm_gbh_af095448_h0t0426.4_EXT”). This alignment indicates there is 82.530% similarity between the two sequences and 78.313% identity. The alignment is shown on the next page in Table 11.

TABLE 11
BestFit Protein Sequence Alignment Analysis

Sequences analyzed:
AF095448.1
pg_mm_gbh_af095448_h0t0426.4_EXT
BESTFIT of:

| | |
|---|---|
| af0954481.seq | check: 6541 from: 1 to: 357 |
| to: | |

pg_mm_gbh_af095448_h0t04264_ext.seq
check: 4062 from: 1 to: 176

| | |
|---|---|
| Percent Similarity: 82.530 | Percent Identity: 78.313 |

| | | |
|---|---|---|
| 3 | TTVPDGCRNGLKSKYYRLCDKAEAWGIVLETVATAGVVTSVAFMLTLPIL | 52 |
| | \|\| \| \|\|\|. \| \|:\|:\|\|\|\| \|\| \|\|\| \|\|\|.\| \| \| .\|\| \| \| \| | |
| 5 | TTAPSGCRSDLDSRYHRLCDLAEGWGIALETLAAVGAVATVACMFALVFL | 54 |
| 53 | VCKVQDSNRRKMLPTQFLFLLGVLGIFGLTFAFIIGLDGSTGPTRFFLFG | 102 |
| | :\|\|\|\|\|\|\|:\|\|\|\|\| \|\|\|\|\|\|\|\|\|\|:\|\|\|\|\|\|\|\|\| \|\|\|.\|\|\|\|\|\|\|\|\|\| | |
| 55 | ICKVQDSNKRKMLPAQFLFLLGVLGVFGLTFAFIIKLDGATGPTRFFLFG | 104 |
| 103 | ILFSICFSCLLAHAVSLTKLVRGRKPLSLLVILGLAVGFSLVQDVIAIEY | 152 |
| | :\|\|.\|\|\|\|\|\|\|\|\|\| .\| \|\|\|\|\|\|\|\|\|\| \|\|\|\| \|\|\|\|\|\|\|\|\|\|\|\|\|\|\|\| | |
| 105 | VLFAICFSCLLAHAFNLIKLVRGRKPLSWLVILSLAVGFSLVQDVIAIEY | 154 |
| 153 | IVLTMNRTNVNVFSEL | 168 |
| | :\|\|\|\|\|\|\|\|\|.\|\|\|\|\| | |
| 155 | LVLTMNRTNVHVFSEL | 170 |

Practicing the Invention

Unless defined otherwise, all technical and scientific terms have the same meaning as is commonly understood by one of skill in the art to which this invention belongs. The definitions set forth below are presented for clarity.

“Isolated,” when referred to a molecule, refers to a molecule that has been identified and separated and/or recovered from a component of its natural environment. Contaminant components of its natural environment are materials that interfere with diagnostic or therapeutic use.

“Probes” are polynucleotide sequences of variable length, preferably between at least about 10 polynucleotides (nt), 100 nt, or many (e.g., 6,000 nt) depending on the specific use. Probes are used to detect identical, similar, or complementary polynucleotide sequences. Longer length probes can be obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies. Probes are substantially purified oligonucleotides that will hybridize under stringent conditions to at least optimally 12, 25, 50, 100, 150, 200, 250, 300, 350 or 400 consecutive sense strand polynucleotide sequence of SEQ ID NOS:1 or 3; or an anti-sense strand polynucleotide sequence of SEQ ID NOS:1 or 3; or of naturally occurring mutants of SEQ ID NOS:1 or 3.

The full- or partial-length native sequence GPCR-like RAIG1 may be used to “pull out” similar (homologous) sequences (Ausubel et al., 1987; Sambrook, 1989), such as: (1) full-length or fragments of GPCR-like RAIG1 cDNA from a cDNA library from any species (e.g. human, murine, feline, canine, bacterial, viral, retroviral, or yeast), (2) from cells or tissues, (3) variants within a species, and (4) homologs, orthologues and variants from other species. To find related sequences that may encode related genes, the probe may be designed to encode unique sequences or degenerate sequences. Sequences may also be GPCR-like RAIG1 genomic sequences including promoters, enhancer elements and introns.

For example, GPCR-like RAIG1 coding region in another species may be isolated using such probes. A probe of about 40 bases is designed, based on mouse GPCR-like RAIG1 (mGPCR-like RAIG1; SEQ ID NO:1), and made. To detect hybridizations, probes are labeled using, for example, radionuclides such as ³²P or ³⁵S, or enzymatic labels such as alkaline phosphatase coupled to the probe via avidin-biotin systems. Labeled probes are used to detect polynucleotides having a complementary sequence to that of mGPCR-like RAIG1 in libraries of cDNA, genomic DNA or mRNA of a desired species.

Probes can be used as a part of a diagnostic test kit for identifying cells or tissues which mis-express a GPCR-like RAIG1, such as by measuring a level of a GPCR-like RAIG1 in a sample of cells from a subject e.g., detecting GPCR-like RAIG1 mRNA levels or determining whether a genomic GPCR-like RAIG1 has been mutated or deleted. Probes are also useful in arrays that allow for the simultaneous examination of multiple sequences.

A polynucleotide is “operably-linked” when placed into a functional relationship with another polynucleotide sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous. Linking can be accomplished by conventional recombinant DNA methods.

“Control sequences” are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

An “isolated polynucleotide” is purified from the setting in which it is naturally found and is separated from at least one contaminant polynucleotide. Isolated GPCR-like RAIG1 polynucleotides are distinguished from the specific GPCR-like RAIG1 nucleotide in cells. However, an isolated GPCR-like RAIG1 polynucleotide includes GPCR-like RAIG1 polynucleotides contained in cells that ordinarily express GPCR-like RAIG1 where, for example, the polynucleotide molecule is in a chromosomal location different from that of natural cells.

In another embodiment, an isolated polynucleotide of the invention comprises a polynucleotide molecule that is a complement of the polynucleotide sequence shown in SEQ ID NOS:1 or 3, or a portion of these sequences (e.g., fragments that can be used as a probes, primers or fragments encoding a biologically-active portion of a GPCR-like RAIG1). A polynucleotide molecule that is “complementary” to the polynucleotide sequence shown in SEQ ID NOS:1 or 3, is one that is sufficiently complementary to the polynucleotide sequence shown in SEQ ID NOS:1 or 3, that it can hydrogen bond with little or no mismatches to the polynucleotide sequence shown in SEQ ID NOS:1 or 3, thereby forming a stable duplex.

“Complementary” refers to Watson-Crick or Hoogsteen base pairing between polynucleotides of a polynucleotide molecule. “Binding” means the physical or chemical interaction between two polypeptides or compounds, associated polypeptides, or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Polynucleotide fragments are at least 6 contiguous polynucleotides or at least 4 contiguous amino acids, a sufficient length to allow for specific hybridization in the case of polynucleotides or for specific recognition of an epitope in the case of amino acids, respectively, and are at most some portion less than a full-length sequence. Fragments may be derived from any contiguous portion of a polynucleotide or amino acid sequence of choice.

“Derivatives” are polynucleotide or amino acid sequences formed from native compounds either directly, by modification or partial substitution. “Analogs” are polynucleotide or amino acid sequences that have a structure similar, but not identical to, the native compound but differ from it in respect to certain components or side chains. Analogs may be synthetic or from a different evolutionary origin and may have a similar or opposite metabolic activity compared to wild type. Homologs are polynucleotide sequences or amino acid sequences of a particular gene that are derived from different species.

Derivatives and analogs may be full length or other than full length if the derivative or analog contains a modified polynucleotide or amino acid. Derivatives or analogs of the polynucleotides or polypeptides of the invention include, but are not limited to, molecules comprising regions that are substantially homologous to GPCR-like RAIG1 or polypeptides by at least about 70%, 80%, or 95% identity (with a preferred identity of 80-95%) over a polynucleotide or amino acid sequence of identical size or when compared to an aligned sequence in which the alignment is done by a well-known algorithm in the art, or whose encoding polynucleotide is capable of hybridizing to the complement of a sequence encoding the aforementioned polypeptides under stringent, moderately stringent, or low stringent conditions (Ausubel et al., 1987).

A “homologous polynucleotide sequence” or “homologous amino acid sequence,” or variations thereof, refer to sequences characterized by a homology at the polynucleotide level or amino acid level as discussed above. Homologous polynucleotide sequences encode those sequences coding for isoforms of GPCR-like RAIG1. Isoforms can be expressed in different tissues of the same organism as a result of, for example, alternative splicing. Alternatively, different genes can encode isoforms such as homologous GPCR-like RAIG1 polynucleotide sequences of species other than mice, including other vertebrates, such as human, frog, rat, rabbit, dog, cat, cow, horse, and other organisms. Homologous polynucleotide sequences also include naturally occurring allelic variations and mutations of SEQ ID NOS:1 or 3. A homologous polynucleotide sequence does not, however, include the exact polynucleotide sequence encoding mouse GPCR-like RAIG1. Homologous polynucleotide sequences may encode conservative amino acid substitutions in SEQ ID NOS:2 or 4, as well as a polypeptide possessing GPCR-like RAIG1 biological activity.

An “open reading frame (ORF)” is a polynucleotide sequence that has a start codon (ATG) and terminates with one of the three “stop” codons (TAA, TAG, or TGA) and encodes a polypeptide or a polypeptide fragment. In this invention, however, an ORF may be any part of a coding sequence that may or may not comprise a start codon and a stop codon. To achieve a unique sequence, preferable GPCR-like RAIG1 ORFs encode at least 50 amino acids.

A GPCR-like RAIG1 can encode a mature GPCR-like RAIG1. A “mature” form of a polypeptide or polypeptide is the product of a naturally occurring polypeptide or precursor form or propolypeptide. The naturally occurring polypeptide, precursor or propolypeptide includes the full-length gene product, encoded by the corresponding genomic sequence or open reading frame. The product “mature” form arises as a result of one or more processing steps as they may take place within the cell or host cell in which the gene product arises. Examples of such processing steps include the cleavage of the N-terminal methionine residue encoded by the initiation codon of an ORF, or the signal peptide cleavage or leader sequence. Thus a mature form arising from a precursor polypeptide or polypeptide that has residues 1 to n, where residue 1 is the N-terminal methionine, would have residues 2 through n after removal of the N-terminal methionine. Alternatively, a mature form arising from a precursor polypeptide or polypeptide having residues 1 to n in which an N-terminal signal sequence from residue 1 to residue m is cleaved, would have the residues from residue m+1 to residue n remaining. A “mature” form of a polypeptide or polypeptide may arise from other post-translational modifications, such as glycosylation, myristoylation or phosphorylation. In general, a mature polypeptide or polypeptide may result from the operation of only one of these processes, or a combination of any of them.

When the molecule is a “purified” polypeptide, the polypeptide will be purified (1) to obtain at least 15 residues of N-terminal or internal amino acid sequence using a sequenator, or (2) to homogeneity by SDS-PAGE under non-reducing or reducing conditions using Coomassie blue or silver stain. Isolated polypeptides include those expressed heterologously in genetically-engineered cells or expressed in vitro, since at least one component of the GPCR-like RAIG1 natural environment is absent. Ordinarily, isolated polypeptides are prepared by at least one purification step.

“Active” GPCR-like RAIG1 or GPCR-like RAIG1 fragment retains a biological and/or an immunological activity of native or naturally-occurring GPCR-like RAIG1. Immunological activity refers to the ability to induce the production of an antibody against an antigenic epitope possessed by a native GPCR-like RAIG1; biological activity refers to a function caused by a native GPCR-like RAIG1 that excludes immunological activity.

An epitope tagged polypeptide refers to a chimeric polypeptide fused to a “tag polypeptide”. Such tags provide epitopes against which Abs can be made or are available, but do not interfere with polypeptide activity. To reduce anti-tag Ab reactivity with endogenous epitopes, the tag polypeptide is preferably unique. Suitable tag polypeptides generally have at least 6 amino acid residues, usually between about 8 and 50 amino acid residues, preferably between 8 and 20 amino acid residues. Examples of epitope tag sequences include HA from Influenza A virus and FLAG.

The invention further encompasses polynucleotide molecules that differ from the polynucleotide sequences shown in SEQ ID NOS:1 or 3, due to degeneracy of the genetic code and thus encode same GPCR-like RAIG1 as that encoded by the polynucleotide sequences shown in SEQ ID NOS:1 or 3. An isolated polynucleotide molecule of the invention has a polynucleotide sequence encoding a polypeptide having an amino acid sequence shown in SEQ ID NOS:2 or 4.

In addition to the GPCR-like RAIG1 sequence shown in SEQ ID NO:1, DNA sequence polymorphisms that change the GPCR-like RAIG1 amino acid sequences may exist within a population. For example, allelic variations among individuals exhibit genetic polymorphisms in GPCR-like RAIG1. The terms “gene” and “recombinant gene” refer to polynucleotide molecules comprising an ORF encoding GPCR-like RAIG1. Such natural allelic variations can typically result in 1-5% variance in GPCR-like RAIG1. Any and all such polynucleotide variations and resulting amino acid polymorphisms in GPCR-like RAIG1, which are the result of natural allelic variation and leave intact GPCR-like RAIG1 functional activity are within the scope of the invention.

Moreover, GPCR-like RAIG1 from other species that have a polynucleotide sequence that differs from the sequence of SEQ ID NOS:1 or 3 are contemplated. polynucleotide molecules corresponding to natural allelic variants and homologs of GPCR-like RAIG1 cDNAs can be isolated based on their homology to SEQ ID NOS:1 or 3 using cDNA-derived probes to hybridize to homologous GPCR-like RAIG1 sequences under stringent conditions.

“GPCR-like RAIG1 variant polynucleotide” or “GPCR-like RAIG1 variant polynucleotide sequence” means a polynucleotide molecule which encodes an active GPCR-like RAIG1 that (1) has at least about 80% polynucleotide sequence identity with a polynucleotide acid sequence encoding a full-length native GPCR-like RAIG1, (2) a full-length native GPCR-like RAIG1 lacking the signal peptide, (3) an extracellular domain of a GPCR-like RAIG1, with or without the signal peptide, or (4) any other fragment of a full-length GPCR-like RAIG1. Ordinarily, a GPCR-like RAIG1 variant polynucleotide will have at least about 80% polynucleotide sequence identity, more preferably at least about 81%-98% polynucleotide sequence identity and yet more preferably at least about 99% polynucleotide sequence identity with the polynucleotide sequence encoding a full-length native GPCR-like RAIG1. A GPCR-like RAIG1 variant polynucleotide may encode full-length native GPCR-like RAIG1 lacking the signal peptide, an extracellular domain of GPCR-like RAIG1, with or without the signal sequence, or any other fragment of a full-length GPCR-like RAIG1. Variants do not encompass the native polynucleotide sequence.

Ordinarily, GPCR-like RAIG1 variants are at least about 30 polynucleotides, often at least about 60, 90, 120, 150, 180, 210, 240, 270, 300, 450, 600 polynucleotides in length, more often at least about 900 polynucleotides in length, or more.

“Percent (%) polynucleotide sequence identity” with respect to GPCR-like RAIG1-encoding polynucleotide sequences is defined as the percentage of polynucleotides in the GPCR-like RAIG1 sequence of interest that are identical with the polynucleotides in a candidate sequence, after aligning the sequences and introducing gaps, if necessary, to achieve the maximum percent sequence identity. Alignment can be achieved in various ways well-known in the art; for instance, using publicly available software such as BLAST, BLAST-2, ALIGN or Megalign (DNASTAR) software. Those skilled in the art can determine appropriate parameters for measuring alignment, including any necessary algorithms to achieve maximal alignment over the full length of the sequences being compared.

When polynucleotide sequences are aligned, the % polynucleotide sequence identity of a given polynucleotide sequence C to, with, or against a given polynucleotide sequence D (which can alternatively be phrased as a given polynucleotide sequence C that has or comprises a certain % polynucleotide sequence identity to, with, or against a given polynucleotide sequence D) can be calculated as:
% polynucleotide sequence identity=W/Z·100

where

W is the number of polynucleotides scored as identical matches by the sequence alignment program's or algorithm's alignment of C and D

and

Z is the total number of polynucleotides in D.

When the length of polynucleotide sequence C is not equal to the length of polynucleotide sequence D, the % polynucleotide sequence identity of C to D will not equal the % polynucleotide sequence identity of D to C.

Homologs or other related sequences (e.g., paralogs) can be obtained by low, moderate or high stringency hybridization with all or a portion of the particular sequence used as a probe using polynucleotide hybridization and cloning methods well known in the art.

The specificity of single stranded DNA to hybridize complementary fragments is determined by the “stringency” of the reaction conditions. Hybridization stringency increases as the propensity to form DNA duplexes decreases. In polynucleotide hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (e.g., homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach to achieve different stringencies is to vary the temperature: higher relative temperatures result in more stringent reaction conditions. Ausubel et al. (1990) provide guidance and an excellent explanation of stringency of hybridization reactions. To hybridize under “stringent conditions” describes hybridization protocols in which polynucleotide sequences at least 60% homologous to each other remain hybridized.

(a) High Stringency

“Stringent hybridization conditions” enable a probe, primer or oligonucleotide to hybridize only to its target sequence. Stringent conditions are sequence-dependent and will differ. Stringent conditions comprise: (1) low ionic strength and high temperature washes (e.g., 15 mM sodium chloride, 1.5 mM sodium citrate, 0.1% sodium dodecyl sulfate at 50° C.); (2) a denaturing agent during hybridization (e.g., 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (sodium choloride/sodium citrate) (0.75 M NaCl, 75 mM sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized.

(b) Moderate Stringency

“Moderately stringent conditions” use washing solutions and hybridization conditions that are less stringent (Sambrook, 1989), such that a polynucleotide will hybridize to the entire, fragments, derivatives or analogs of SEQ ID NOS:1, 6 or 11. One example comprises hybridization in 6×SSC, 5× Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions are described (Ausubel et al., 1987; Kriegler, 1990).

(c) Low Stringency

“Low stringent conditions” use washing solutions and hybridization conditions that are less stringent than those for moderate stringency (Sambrook, 1989), such that a polynucleotide will hybridize to the entire, fragments, derivatives or analogs of SEQ ID NOS:1 or 3. An example of low stringency hybridization conditions is hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations have been well described (Ausubel et al., 1987; Kriegler, 1990; Shilo and Weinberg, 1981).

In addition to naturally-occurring allelic variants of GPCR-like RAIG1, changes can be introduced by mutation into SEQ ID NO:1 that incur alterations in the amino acid sequence of GPCR-like RAIG1 but does not alter GPCR-like RAIG1 function. For example, polynucleotide substitutions leading to amino acid substitutions at “non-essential” amino acid residues can be made in SEQ ID NOS:2. A “non-essential” amino acid residue is a residue that can be altered from the wild-type sequence of GPCR-like RAIG1 without altering GPCR-like RAIG1 biological activity, whereas an “essential” amino acid residue is required for biological activity. For example, amino acid residues that are conserved among the GPCR-like RAIG amino acids of the invention are particularly non-amenable to alteration (Table 9).

Useful conservative substitutions are shown in Table A, “Preferred substitutions.” Conservative substitutions whereby an amino acid of one class is replaced with another amino acid of the same type fall within the scope of the subject invention so long as the substitution does not materially alter the biological activity of the compound. If such substitutions result in a change in biological activity, then more substantial changes, indicated in Table B as exemplary, are introduced and the products screened for GPCR-like RAIG1 biological activity.

TABLE A
Preferred substitutions

| Original | | Preferred |
|---|---|---|
| residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, | Ile |
| | Phe | |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

Non-conservative substitutions that affect (1) the structure of the polypeptide backbone, such as a β-sheet or α-helical conformation, (2) the charge or (3) hydrophobicity, or (4) the bulk of the side chain of the target site can modify GPCR-like RAIG1 polypeptide function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino acid classes

| Class | Amino acids |
|---|---|
| hydrophobic | Norleucine, Met, Ala, Val, Leu, |
| | Ile |
| neutral hydrophilic | Cys, Ser, Thr |
| acidic | Asp, Glu |
| basic | Asn, Gln, His, Lys, Arg |
| disrupt chain | Gly, Pro |
| conformation | |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce GPCR-like RAIG1 variants (Ausubel et al., 1987; Sambrook, 1989).

Using antisense and sense GPCR-like RAIG1 oligonucleotides can prevent GPCR-like RAIG1 expression. Antisense or sense oligonucleotides are singe-stranded polynucleotides, either RNA or DNA, which can bind target GPCR-like RAIG1 mRNA (sense) or DNA (antisense) sequences. Anti-sense polynucleotides can be designed according to Watson and Crick or Hoogsteen base pairing rules. The anti-sense polynucleotide molecule can be complementary to the entire coding region of GPCR-like RAIG1 mRNA, but more preferably to only a portion of the coding or noncoding region of GPCR-like RAIG1 mRNA. For example, the anti-sense oligonucleotide can be complementary to the region surrounding the translation start site of GPCR-like RAIG1 mRNA. Antisense or sense oligonucleotides may comprise a fragment of the GPCR-like RAIG1 coding region of at least about 14 polynucleotides, preferably from about 14 to 30 polynucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Methods to derive antisense or sense oligonucleotides are well described (Stein and Cohen, 1988; van der Krol et al., 1988a).

Examples of modified polynucleotides that can be used to generate the anti-sense polynucleotide include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the anti-sense polynucleotide can be produced using an expression vector into which a polynucleotide has been sub-cloned in an anti-sense orientation such that the transcribed RNA will be complementary to a target polynucleotide of interest.

To introduce antisense or sense oligonucleotides into target cells (cells containing a target polynucleotide sequence), any gene transfer method may be used. Examples of gene transfer methods include (1) biological, such as gene transfer vectors like Epstein-Barr virus or conjugating the exogenous DNA to a ligand-binding molecule, (2) physical, such as electroporation and injection, and (3) chemical, such as CaPO₄ precipitation and oligonucleotide-lipid complexes.

An antisense or sense oligonucleotide is inserted into a suitable gene transfer vector, such as a retroviral vector. A cell containing the target polynucleotide sequence is contacted with the recombinant vector, either in vivo or ex vivo. For example, suitable retroviral vectors include those derived from the murine retrovirus M-MuLV, N2 (a retrovirus derived from M-MuLV), or the double copy vectors designated DCT5A, DCT5B and DCT5C (WO 90/13641, 1990). To achieve sufficient polynucleotide molecule transcription, vector constructs in which the transcription of the anti-sense polynucleotide molecule is controlled by a strong pol II or pol III promoter are preferred. Also preferred are tissue- and cell-specific promoters, when known.

To specify target cells in a mixed population of cells, cell surface receptors that are specific to the target cells can be exploited. Antisense and sense oligonucleotides are conjugated to a ligand-binding molecule, as described (WO 91/04753, 1991). Examples of suitable ligand-binding molecules include cell surface receptors, growth factors, cytokines, or other ligands that bind to target cell surface molecules. Preferably, conjugation of the ligand-binding molecule does not substantially interfere with the ability of the receptors or molecules to bind the ligand-binding molecule conjugate, or block entry of the sense or antisense oligonucleotide or its conjugated version into the cell.

Liposomes efficiently transfer sense or an antisense oligonucleotide to cells (WO 90/10448, 1990). The sense or antisense oligonucleotide-lipid complex is preferably dissociated within the cell by an endogenous lipase.

The anti-sense polynucleotide molecule of the invention may be an α-anomeric polynucleotide molecule. An α-anomeric polynucleotide molecule forms specific double-stranded hybrids with complementary RNA in which, contrary to the usual α-units, the strands run parallel to each other (Gautier et al., 1987). The anti-sense polynucleotide molecule can also comprise a 2′-o-methylribonucleotide (Inoue et al., 1987a) or a chimeric RNA-DNA analog (Inoue et al., 1987b).

An anti-sense polynucleotide may be a catalytic RNA molecule with ribonuclease activity, a ribozyme. For example, hammerhead ribozymes (Haseloff and Gerlach, 1988) can be used to catalytically-cleave GPCR-like RAIG1 mRNA transcripts and thus inhibit translation. A ribozyme specific for a GPCR-like RAIG1-encoding polynucleotide can be designed based on the polynucleotide sequence of a GPCR-like RAIG1 cDNA (i.e., SEQ ID NO:1). For example, a derivative of a Tetrahymena L-19 IVS RNA can be constructed in which the polynucleotide sequence of the active site is complementary to the polynucleotide sequence to be cleaved in a GPCR-like RAIG1-encoding mRNA (Cech et al., U.S. Pat. No. 5,116,742, 1992; Cech et al., U.S. Pat. No. 4,987,071, 1991). GPCR-like RAIG1 mRNA can also be used to select a catalytic RNA having a specific ribonuclease activity from a pool of RNA molecules (Bartel and Szostak, 1993).

Alternatively, GPCR-like RAIG1 expression can be inhibited by targeting polynucleotide sequences complementary to the regulatory region of a GPCR-like RAIG1 (e.g., GPCR-like RAIG1 promoter and/or enhancers) to form triple helical structures that prevent transcription of the GPCR-like RAIG1 in target cells (Helene, 1991; Helene et al., 1992; Maher, 1992).

Modifications of antisense and sense oligonucleotides can augment their effectiveness. Modified sugar-phosphodiester bonds or other sugar linkages (WO 91/06629, 1991) increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

For example, the deoxyribose phosphate backbone can be modified to generate peptide polynucleotides (Hyrup and Nielsen, 1996). “Peptide polynucleotides” (PNAs) refer to polynucleotide mimics in that the deoxyribose phosphate backbone is replaced by a pseudopeptide backbone, and only the four natural nucleobases are retained. The neutral backbone of PNAs allows for specific hybridization to DNA and RNA under conditions of low ionic strength. PNA oligomers can be synthesized using solid phase peptide synthesis protocols (Hyrup and Nielsen, 1996; Perry-O'Keefe et al., 1996).

PNAs of GPCR-like RAIG1 can be used in therapeutic and diagnostic applications. For example, PNAs can be used as anti-sense or antigene agents for sequence-specific modulation of gene expression by inducing transcription or translation arrest or inhibiting replication. GPCR-like RAIG1 PNAs may also be used in the analysis of single base pair mutations (e.g., PNA directed PCR clamping); as artificial restriction enzymes when used in combination with other enzymes, e.g., S₁ nucleases (Hyrup and Nielsen, 1996); or as probes or primers for DNA sequence and hybridization (Hyrup and Nielsen, 1996; Perry-O'Keefe et al., 1996).

GPCR-like RAIG1 PNAs can be modified to enhance their stability or cellular uptake. Lipophilic or other helper groups may be attached to PNAs, PNA-DNA dimers formed, or the use of liposomes or other drug delivery techniques. For example, PNA-DNA chimeras can be generated that combine the advantages of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation (Hyrup and Nielsen, 1996). The synthesis of PNA-DNA chimeras have been described (Finn et al., 1996; Hyrup and Nielsen, 1996). For example, a DNA chain can be synthesized on a solid support using standard phosphoramidite coupling chemistry, and modified nucleoside analogs, e.g. 5′-(4-methoxytrityl)amino-5′-deoxy-thymidine phosphoramidite, can be used between the PNA and the 5′ end of DNA (Finn et al., 1996; Hyrup and Nielsen, 1996). PNA monomers are then coupled in a stepwise manner to produce a chimeric molecule with a 5′ PNA segment and a 3′ DNA segment (Finn et al., 1996). Alternatively, chimeric molecules can be synthesized with a 5′ DNA segment and a 3′ PNA segment (Petersen et al., 1976).

The oligonucleotide may include other appended groups such as peptides (e.g., for targeting host cell receptors in vivo), or agents facilitating transport across the cell membrane (Lemaitre et al., 1987; Letsinger et al., 1989) or the blood-brain barrier (Pardridge and Schimmel, WO89/10134, 1989). In addition, oligonucleotides can be modified with hybridization-triggered cleavage agents (van der Krol et al., 1988b) or intercalating agents (Zon, 1988). The oligonucleotide may be conjugated to another molecule, e.g., a peptide, a hybridization triggered cross-linking agent, a transport agent, a hybridization-triggered cleavage agent, and the like.

GPCR-Like RAIG1 Polypeptides

The invention pertains to isolated GPCR-like RAIG, and biologically-active portions, derivatives, fragments, analogs or homologs thereof. Also provided are polypeptide fragments suitable for use as immunogens to raise anti-GPCR-like RAIG1 Abs. GPCR-like RAIG1 may be isolated from cells and tissues, produced by recombinant DNA techniques or chemically synthesized.

A GPCR-like RAIG1 polypeptide includes an amino acid sequence provided in SEQ ID NO:2. The invention also includes mutant or variant polypeptides any of whose residues may be changed from the corresponding residues shown in SEQ ID NO:2 while still encoding active GPCR-like RAIG1, or a functional fragment.

In general, a GPCR-like RAIG1 variant that preserves GPCR-like RAIG1-like function and includes any variant in which residues at a particular position in the sequence have been substituted by other amino acids, and further includes the possibility of inserting an additional residue or residues between two residues of the parent polypeptide as well as the possibility of deleting one or more residues from the parent sequence. Preferably, the substitution is a conservative substitution (Table A).

“GPCR-like RAIG1 polypeptide variant” means an active GPCR-like RAIG1 having at least: (1) about 80% amino acid sequence identity with a full-length native GPCR-like RAIG1 sequence, (2) a GPCR-like RAIG1 sequence lacking a signal peptide, (3) an extracellular domain of a GPCR-like RAIG1, with or without a signal peptide, or (4) any other fragment of a full-length GPCR-like RAIG1 sequence. For example, GPCR-like RAIG1 variants include those wherein one or more amino acid residues are added or deleted at the N- or C-terminus of the full-length native amino acid sequence. A GPCR-like RAIG1 polypeptide variant will have at least about 80% amino acid sequence identity, preferably at least about 81% amino acid sequence identity, more preferably at least about 82%-98% amino acid sequence identity and most preferably at least about 99% amino acid sequence identity with a full-length native sequence GPCR-like RAIG1 sequence. Ordinarily, GPCR-like RAIG1 variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in a GPCR-like RAIG1 sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Publicly available computer software such as BLAST, BLAST2, ALIGN2 or Megalign (DNASTAR) can be used to align polypeptide sequences. Those skilled in the art will determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:
% amino acid sequence identity=X/Y·100

where

X is the number of amino acid residues scored as identical matches by the sequence alignment program's or algorithm's alignment of A and B

and

Y is the total number of amino acid residues in B.

If the length of amino acid sequence A is not equal to the length of amino acid sequence B, the % amino acid sequence identity of A to B will not equal the % amino acid sequence identity of B to A.

An “isolated” or “purified” polypeptide, or biologically active fragment is separated and/or recovered from a component of its natural environment. Contaminant components include materials that would typically interfere with diagnostic or therapeutic uses for the polypeptide, such as enzymes, hormones, and other polypeptideaceous or non-polypeptideaceous materials. Preferably, the polypeptide is purified to a sufficient degree to obtain at least 15 residues of N-terminal or internal amino acid sequence. To be substantially isolated, preparations have less than 30% by dry weight of contaminants, more preferably less than 20%, 10% and most preferably less than 5% contaminants. An isolated, recombinantly-produced GPCR-like RAIG1 or biologically active portion is preferably substantially free of culture medium, i.e., culture medium represents less than 20%, more preferably less than about 10%, and most preferably less than about 5% of the volume of the GPCR-like RAIG1 preparation. Examples of contaminants include cell debris, culture media, and substances used and produced during in vitro synthesis of GPCR-like RAIG1.

Biologically active portions of GPCR-like RAIG1 include peptides comprising amino acid sequences sufficiently homologous to, or derived from, the amino acid sequences of GPCR-like RAIG1 (SEQ ID NO:2) that include fewer amino acids than the full-length GPCR-like RAIG1, and exhibit at least one activity of a GPCR-like RAIG1. Biologically active portions comprise a domain or motif with at least one activity of native GPCR-like RAIG1. A biologically active portion of a GPCR-like RAIG1 can be a polypeptide that is 10, 25, 50, 100 or more amino acid residues in length. Other biologically active portions, in which other regions of the polypeptide are deleted, can be prepared by recombinant techniques and evaluated for one or more of the functional activities of a native GPCR-like RAIG1.

Biologically active portions of a GPCR-like RAIG1 may have an amino acid sequence shown in SEQ ID NO:2, or be substantially homologous to SEQ ID NO:2, and retains the functional activity of the polypeptide of SEQ ID NO:2, yet differs in amino acid sequence due to natural allelic variation or mutagenesis. Other biologically active GPCR-like RAIG1 may comprise an amino acid sequence at least 45% homologous to the amino acid sequence of SEQ ID NO:2, and retains the functional activity of native GPCR-like RAIG1. Homology can be determined as described in GPCR-like RAIG1 polypeptide variants, above.

Fusion polypeptides are useful in expression studies, cell-localization, bioassays, and GPCR-like RAIG1 purification. A GPCR-like RAIG1 “chimeric polypeptide” or “fusion polypeptide” comprises GPCR-like RAIG1 fused to a non-GPCR-like RAIG1 polypeptide. A non-GPCR-like RAIG1 polypeptide is not substantially homologous to GPCR-like RAIG1 (SEQ ID NO:2). A GPCR-like RAIG1 fusion polypeptide may include any portion to an entire GPCR-like RAIG1, including any number of biologically active portions. In some host cells, heterologous signal sequence fusions may ameliorate GPCR-like RAIG1 expression and/or secretion. Exemplary fusions are presented in Table C.

Other fusion partners can adapt GPCR-like RAIG1 therapeutically. Fusions with members of the immunoglobulin (Ig) family are useful to inhibit GPCR-like RAIG1 ligand or substrate interactions, consequently suppressing GPCR-like RAIG1-mediated signal transduction in vivo. GPCR-like RAIG1-Ig fusion polypeptides can also be used as immunogens to produce anti-GPCR-like Abs in a subject, to purify GPCR-like RAIG1 ligands, and to screen for molecules that inhibit interactions of GPCR-like RAIG1 with other molecules.

Fusion polypeptides can be easily created using recombinant methods. A polynucleotide encoding GPCR-like RAIG1 can be fused in-frame with a non-GPCR-like RAIG1 encoding polynucleotide, to the GPCR-like RAIG1 N- or C-terminus, or internally. Fusion genes may also be synthesized by conventional techniques, including automated DNA synthesizers and PCR amplification using anchor primers that give rise to complementary overhangs between two consecutive gene fragments that can subsequently be annealed and reamplified to generate a chimeric gene sequence (Ausubel et al., 1990). Many vectors are commercially available that facilitate sub-cloning GPCR-like RAIG1 in-frame to a fusion moiety.

TABLE C
Useful non-GPCR-like RAIG1 fusion polypeptides and usefulness thereof

| Polypeptide | in vitro | in vivo | Notes | Reference |
|---|---|---|---|---|
| Human growth | Radioimmunoassay | none | Expensive, | (Selden et al., |
| hormone (hGH) | | | insensitive, | 1986) |
| | | | narrow linear | |
| | | | range. | |
| β-glucuronidase | Colorimetric, | colorimetric | sensitive, | (Gallagher, |
| (GUS) | fluorescent, or | (histo-chemical | broad linear | 1992) |
| | chemiluminescent | staining with X- | range, non- | |
| | | gluc) | iostopic. | |
| Green | Fluorescent | fluorescent | can be used in | (Chalfie et al., |
| fluorescent | | | live cells; | 1994) |
| polypeptide | | | resists photo- | |
| (GFP) and | | | bleaching | |
| related | | | | |
| molecules (RFP, | | | | |
| BFP, YFP, etc.) | | | | |
| Luciferase | bioluminsecent | Bioluminescent | polypeptide is | (de Wet et al., |
| (firefly) | | | unstable, | 1987) |
| | | | difficult to | |
| | | | reproduce, | |
| | | | signal is brief | |
| Chloramphenicoal | Chromatography, | none | Expensive | (Gorman et al., |
| acetyltransferase | differential | | radioactive | 1982) |
| (CAT) | extraction, | | substrates, | |
| | fluorescent, or | | time- | |
| | immunoassay | | consuming, | |
| | | | insensitive, | |
| | | | narrow linear | |
| | | | range | |
| β-galacto-sidase | colorimetric, | colorimetric | sensitive, | (Alam and |
| | fluorescence, | (histochemical | broad linear | Cook, 1990) |
| | chemiluminscence | staining with X- | range; some | |
| | | gal), bioluminescent | cells have high | |
| | | in | endogenous | |
| | | live cells | activity | |
| Secrete alkaline | colorimetric, | none | Chemiluminscence | (Berger et al., |
| phosphatase | bioluminescent, | | assay is | 1988) |
| (SEAP) | chemiluminescent | | sensitive and | |
| | | | broad linear | |
| | | | range; some | |
| | | | cells have | |
| | | | endogenouse | |
| | | | alkaline | |
| | | | phosphatase | |
| | | | activity | |

Therapeutic Applications of GPCR-Like RAIG1

“Antagonist” includes any molecule that partially or fully blocks, inhibits, or neutralizes a biological activity of an endogenous GPCR-like RAIG1. Similarly, “agonist” includes any molecule that mimics a biological activity of an endogenous GPCR-like RAIG1. Molecules that can act as agonists or antagonists include Abs or antibody fragments, fragments or variants of endogenous GPCR-like RAIG1, peptides, antisense oligonucleotides, small organic molecules, etc.

To assay for antagonists, a GPCR-like RAIG1 is added to, or expressed in, a cell along with the compound to be screened for a particular activity. If the compound inhibits the activity of interest in the presence of the GPCR-like RAIG1, that compound is an antagonist to the GPCR-like RAIG1; if GPCR-like RAIG1 activity is enhanced, the compound is an agonist.

GPCR-like RAIG1-expressing cells are easily identified using standard methods. For example, antibodies that recognize the amino- or carboxy-terminus of a GPCR-like RAIG1 can be used to screen candidate cells by immunoprecipitation, Western blots, and immunohistochemical techniques. Likewise, SEQ ID NO:1 can be used to design primers and probes that detect a GPCR-like RAIG1 mRNA in cells or samples from cells.

(a) Examples of Potential Antagonists and Agonist

Examples of antagonists and agonists include: (1) small organic and inorganic compounds, (2) small peptides, (3) Abs and derivatives, (4) polypeptides closely related to GPCR-like RAIG1, (5) antisense DNA and RNA, (6) ribozymes, (7) triple DNA helices and (8) polynucleotide aptamers.

Small molecules that bind to the GPCR-like RAIG1 active site or other relevant part of the polypeptide and inhibit the biological activity of a GPCR-like RAIG1 are antagonists. Examples of small molecule antagonists include small peptides, peptide-like molecules, preferably soluble, and synthetic non-peptidyl organic or inorganic compounds. These same molecules, if they enhance GPCR-like RAIG1 activity, are examples of agonists.

Almost any antibody that affects a GPCR-like RAIG1 function is a candidate antagonist, and occasionally, agonist. Examples of antibody antagonists include polyclonal, monoclonal, single-chain, anti-idiotypic, chimeric Abs, or humanized versions of such Abs or fragments. Abs may be from any species in which an immune response can be raised. Humanized Abs are also contemplated.

Alternatively, a potential antagonist or agonist may be a closely related polypeptide, for example, a mutated form of the GPCR-like RAIG1 that recognizes a GPCR-like RAIG1-interacting polypeptide but imparts no effect other than competitively inhibiting GPCR-like RAIG1 action. Alternatively, a mutated GPCR-like RAIG1 can be constitutively activated and act as an agonist.

Antisense RNA or DNA constructs can be effective antagonists. Antisense RNA or DNA molecules block function by inhibiting translation by hybridizing to targeted mRNA. Antisense technology can be used to control gene expression through triple-helix formation or antisense DNA or RNA, both of which depend on polynucleotide binding to DNA or RNA. For example, the 5′ coding portion of a GPCR-like RAIG1 sequence is used to design an antisense RNA oligonucleotide of from about 10 to 40 base pairs in length. A DNA oligonucleotide is designed to be complementary to a region of the gene involved in transcription (triple helix) (Beal and Dervan, 1991; Cooney et al., 1988; Lee et al., 1979), thereby preventing transcription and the production of a GPCR-like RAIG1. The antisense RNA oligonucleotide hybridizes to the mRNA in vivo and blocks translation of the mRNA molecule (antisense) (Cohen, 1989; Okano et al., 1991). These oligonucleotides can also be delivered to cells such that the antisense RNA or DNA may be expressed in vivo to inhibit production of GPCR-like RAIG1. When antisense DNA is used, oligodeoxyribonucleotides derived from the translation-initiation site, e.g., between about −10 and +10 positions of the target gene polynucleotide sequence, are preferred.

To inhibit transcription, triple-helix polynucleotides that are single-stranded and comprise deoxynucleotides are useful antagonists. These oligonucleotides are designed such that triple-helix formation via Hoogsteen base-pairing rules is promoted, generally requiring stretches of purines or pyrimidines (WO 97/33551, 1997).

Aptamers are short oligonucleotide sequences that recognize and specifically bind almost any type of molecule. The systematic evolution of ligands by exponential enrichment (SELEX) process (Ausubel et al., 1987; Ellington and Szostak, 1990; Tuerk and Gold, 1990) is a powerful technique to identify aptamers. Aptamers have many diagnostic and clinical uses; almost any use in which an antibody is employed clinically or diagnostically, aptamers too may be used. Aptamers can be easily applied to a variety of formats, including administration in pharmaceutical compositions, in bioassays, and diagnostic tests (Jayasena, 1999).

Anti-GPCR-Like RAIG1 Abs

The invention encompasses Abs and Ab fragments, such as F_{ab} or (F_{ab})₂, that bind immunospecifically to any epitope of a GPCR-like RAIG1 molecule.

“Antibody” (Ab) comprises Abs directed against a GPCR-like RAIG1 (an anti-GPCR-like RAIG1 Ab; including agonist, antagonist, and neutralizing Abs), anti-GPCR-like RAIG1 Ab compositions with poly-epitope specificity, single chain anti-GPCR-like RAIG1 Abs, and fragments of anti-GPCR-like RAIG1 Abs. A “monoclonal Ab” is obtained from a population of substantially homogeneous Abs, i.e., the individual Abs comprising the population are identical except for possible naturally-occurring mutations that may be present in minor amounts. Exemplary Abs include polyclonal (pAb), monoclonal (mAb), humanized, bi-specific (bsAb), and heteroconjugate Abs.

Polyclonal Abs (pAbs)

Polyclonal Abs can be raised in a mammalian host by one or more injections of an immunogen and, if desired, an adjuvant. Typically, the immunogen (and adjuvant) is injected in the mammal by multiple subcutaneous or intraperitoneal injections. The immunogen may include a GPCR-like RAIG1 or a GPCR-like RAIG1 fusion polypeptide. Examples of adjuvants include Freund's complete and monophosphoryl Lipid A synthetic-trehalose dicorynomycolate (MPL-TDM). To improve the immune response, an immunogen may be conjugated to a polypeptide that is immunogenic in the host, such as keyhole limpet hemocyanin (KLH), serum albumin, bovine thyroglobulin, and soybean trypsin inhibitor. Protocols for antibody production are well-known (Ausubel et al., 1990; Harlow and Lane, 1988). Alternatively, pAbs may be made in chickens, producing IgY molecules (Schade et al., 1996).

Monoclonal Abs (mAbs)

Anti-GPCR-like RAIG1 mAbs may be prepared using hybridoma methods (Milstein and Cuello, 1983). Hybridoma methods comprise at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-GPCR-like RAIG1) mAb.

A rat, guinea pig, hamster, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other sources are preferred. The immunogen typically includes GPCR-like RAIG1 or GPCR-like RAIG1 fusion polypeptide.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used, or rat or mouse myeloma cell lines. Because pure populations of hybridoma cells and not unfused immortalized cells are preferred, after fusion, the cells are grown in a suitable medium that inhibits the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient cells while permitting hybridomas to grow.

Preferred immortalized cells fuse efficiently; can be isolated from mixed populations by selecting in a medium such as HAT; and support stable and high-level expression of antibody after fusion. Preferred immortalized cell lines are murine myeloma lines, available from the American Type Culture. Collection (Manassas, Va.). Human myeloma and mouse-human heteromyeloma cell lines also have been described for the production of human mAbs (Kozbor et al., 1984; Schook, 1987).

Because hybridoma cells secrete antibody, the culture media can be assayed for mAbs directed against GPCR-like RAIG1 (anti-GPCR-like RAIG1 mAbs). Immunoprecipitation or in vitro binding assays, such as radio immunoassay (RIA) or enzyme-linked immunoabsorbent assays (ELISA), measure the binding specificity of mAbs (Harlow and Lane, 1988; Harlow and Lane, 1999), including Scatchard analysis (Munson and Rodbard, 1980).

Anti-GPCR-like RAIG1 mAb secreting hybridoma cells may be isolated as single clones by limiting dilution procedures and sub-cultured (Goding, 1996). Suitable culture media include Dulbecco's Modified Eagle's Medium, RPMI-1640, or if desired, a polypeptide-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as polypeptide A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also be made by recombinant methods (U.S. Pat. No. 4,166,452, 1979). DNA encoding anti-GPCR-like RAIG1 mAbs can be readily isolated and sequenced using conventional procedures, e.g., using oligonucleotide probes that specifically bind to murine heavy and light antibody chain genes, to probe preferably DNA isolated from anti-GPCR-like RAIG1-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig polypeptide, to express mAbs. The isolated DNA fragments can be modified by substituting the coding sequence for human heavy and light chain constant domains in place of the homologous murine sequences (U.S. Pat. No. 4,816,567, 1989; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigen-combining site to create a chimeric bivalent antibody.

Monovalent Abs

The Abs may be monovalent Abs, and thus will not cross-link. One method involves recombinant expression of Ig light chain and modified heavy chain. Heavy chain truncations generally at any point in the F_{c} region will prevent heavy chain cross-linking. Alternatively, the relevant cysteine residues are substituted with another amino acid residue or are deleted, preventing crosslinking by disulfide binding. In vitro methods are also suitable for preparing monovalent Abs. Abs can be digested to produce fragments, such as F_{ab} (Harlow and Lane, 1988; Harlow and Lane, 1999).

Humanized and Human Abs

Humanized forms of non-human Abs that bind a GPCR-like RAIG1 are chimeric Igs, Ig chains or fragments (such as Fᵥ, F_{ab}, F_{ab′}, F_{(ab′)2} or other antigen-binding subsequences of Abs) that contains minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” Abs are chimeric Abs (U.S. Pat. No. 4,816,567, 1989), wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fᵥ framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (F_{c}), typically that of a human Ig (Jones et al., 1986; Presta, 1992; Riechmann et al., 1988).

Human Abs can also be produced using various techniques, including phage display libraries (Hoogenboom et al., 1991; Marks et al., 1991) and human mAbs (Boerner et al., 1991; Reisfeld and Sell, 1985). Introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire (U.S. Pat. No. 5,545,807, 1996; U.S. Pat. No. 5,569,825, 1996; U.S. Pat. No. 5,633,425, 1997; U.S. Pat. No. 5,661,016, 1997; U.S. Pat. No. 5,625,126, 1997; Fishwild et al., 1996; Lonberg and Huszar, 1995; Lonberg et al., 1994; Marks et al., 1992).

Bi-Specific mAbs

Bi-specific Abs are monoclonal, preferably human or humanized, that have binding specificities for at least two different antigens. For example, a binding specificity is a GPCR-like RAIG1; the other is for any antigen of choice, preferably a cell-surface polypeptide or receptor or receptor subunit.

The recombinant production of bi-specific Abs is often achieved by co-expressing two Ig heavy-chain/light-chain pairs, each having different specificities (Milstein and Cuello, 1983). The random assortment of these Ig heavy and light chains in the resulting hybridomas (quadromas) produce a potential mixture of ten different antibody molecules, of which only one has the desired bi-specific structure. The desired antibody can be purified using affinity chromatography or other techniques (WO 93/08829, 1993; Traunecker et al., 1991).

To manufacture a bi-specific antibody (Suresh et al., 1986), variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. DNAs encoding the Ig heavy-chain fusions and, if desired, the Ig light chain, are inserted into separate expression vectors and are co-transfected into a suitable host organism.

The interface between a pair of antibody molecules can be engineered to maximize the percentage of heterodimers that are recovered from recombinant cell culture (WO 96/27011, 1996). The preferred interface comprises at least part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This mechanism increases the yield of the heterodimer over unwanted end products such as homodimers.

Bi-specific Abs can be prepared as full length Abs or antibody fragments (e.g. F_{(ab′)2} bi-specific Abs). One technique to generate bi-specific Abs exploits chemical linkage. Intact Abs can be proteolytically cleaved to generate F_{(ab′)2} fragments (Brennan et al., 1985). Fragments are reduced with a dithiol complexing agent, such as sodium arsenite, to stabilize vicinal dithiols and prevent intermolecular disulfide formation. The generated F_{ab′} fragments are then converted to thionitrobenzoate (TNB) derivatives. One of the Fab_{ab′}-TNB derivatives is then reconverted to the F_{ab′}-thiol by reduction with mercaptoethylamine and is mixed with an equimolar amount of the other F_{ab′}-TNB derivative to form the bi-specific antibody. The produced bi-specific Abs can be used as agents for the selective immobilization of enzymes.

F_{ab′} fragments may be directly recovered from E. coli and chemically coupled to form bi-specific Abs. For example, fully humanized bi-specific F_{(ab′)2} Abs can be produced (Shalaby et al., 1992). Each F_{ab′} fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited (Kostelny et al., 1992). Peptides from the Fos and Jun polypeptides are linked to the F_{ab′} portions of two different Abs by gene fusion. The antibody homodimers are reduced at the hinge region to form monomers and then re-oxidized to form antibody heterodimers. This method can also produce antibody homodimers. “Diabody” technology (Holliger et al., 1993) provides an alternative method to generate bi-specific antibody fragments. The fragments comprise a heavy-chain variable domain (V_{H}) connected to a light-chain variable domain (V_{L}) by a linker that is too short to allow pairing between the two domains on the same chain. The V_{H} and V_{L} domains of one fragment are forced to pair with the complementary V_{L} and V_{H} domains of another fragment, forming two antigen-binding sites. Another strategy for making bi-specific antibody fragments is the use of single-chain Fᵥ (sFᵥ) dimers (Gruber et al., 1994). Abs with more than two valencies may also be made, such as tri-specific Abs (Tutt et al., 1991).

Exemplary bi-specific Abs may bind to two different epitopes on a given GPCR-like RAIG1. Alternatively, cellular defense mechanisms can be restricted to a particular cell expressing the particular GPCR-like RAIG1: an anti-GPCR-like RAIG1 arm may be combined with an arm that binds to a leukocyte triggering molecule, such as a T-cell receptor molecule (e.g. CD2, CD3, CD28, or B7), or to F_{c} receptors for IgG (F_{c}γR), such as F_{c}γRI (CD64), F_{c}γRII (CD32) and F_{c}γRIII (CD16). Bi-specific Abs may also be used to target cytotoxic agents to cells that express a particular GPCR-like RAIG1. These Abs possess a GPCR-like RAIG1-binding arm and an arm that binds a cytotoxic agent or a radionuclide chelator.

Heteroconjugate Abs

Heteroconjugate Abs, consisting of two covalently joined Abs, target immune system cells to dispose unwanted cells (U.S. Pat. No. 4,676,980, 1987) and for treatment of human immunodeficiency virus (HIV) infection (WO 91/00360, 1991; WO 92/20373, 1992). Abs prepared in vitro using synthetic polypeptide chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate (U.S. Pat. No. 4,676,980, 1987).

Immunoconjugates

Immunoconjugates may comprise an antibody conjugated to a cytotoxic agent such as a chemotherapeutic agent, toxin (e.g., an enzymatically active toxin or fragment of bacterial, fungal, plant, or animal origin), or a radioactive isotope (i.e., a radioconjugate).

Useful enzymatically-active toxins and fragments include Diphtheria A chain, non-binding active fragments of Diphtheria toxin, exotoxin A chain from Pseudomonas aeruginosa, ricin A chain, abrin A chain, modeccin A chain, α-sarcin, Aleurites fordii polypeptides, Dianthin polypeptides, Phytolaca americana polypeptides, Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated Abs, such as ²¹²Bi, ¹³¹I, ¹³¹In, ⁹⁰Y, and ¹⁸⁶Re.

Conjugates of the antibody and cytotoxic agent are made using a variety of bi-functional polypeptide-coupling agents, such as N-succinimidyl-3-(2-pyridyldithiol) propionate (SPDP), iminothiolane (IT), bi-functional derivatives of imidoesters (such as dimethyl adipimidate HCl), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared (Vitetta et al., 1987). ¹⁴C-labeled 1-isothiocyanatobenzyl-3-methyldiethylene triaminepentaacetic acid (MX-DTPA) is an exemplary chelating agent for conjugating radionuclide to antibody (WO 94/11026, 1994).

The antibody may be conjugated to a “receptor” (such as streptavidin) to use in tumor pre-targeting, wherein the antibody-receptor conjugate is administered to the patient, followed by removal of unbound conjugate from the circulation using a clearing agent and then administration of a streptavidin “ligand” (e.g., biotin) that is conjugated to a cytotoxic agent (e.g., a radionuclide).

Effector Function Engineering

Antibodies can be modified to enhance their effectiveness in treating a disease, such as obesity, to target and kill adipose cells. For example, cysteine residue(s) may be introduced into the F_{c} region, thereby allowing interchain disulfide bond formation in this region. Such homodimeric Abs often have improved internalization capability and/or increased complement-mediated cell killing and antibody-dependent cellular cytotoxicity (Caron et al., 1992; Shopes, 1992). Homodimeric Abs with enhanced activity can be prepared using hetero-bifunctional cross-linkers (Wolff et al., 1993). Alternatively, an antibody engineered with dual F_{c} regions may have enhanced complement lysis (Stevenson et al., 1989).

Immunoliposomes

Liposomes containing Abs (immunoliposomes) may also be formulated (U.S. Pat. No. 4,485,045, 1984; U.S. Pat. No. 4,544,545, 1985; U.S. Pat. No. 5,013,556, 1991; Eppstein et al., 1985; Hwang et al., 1980). Useful liposomes can be generated by a reverse-phase evaporation method with a lipid composition comprising phosphatidylcholine, cholesterol, and PEG-derivatized phosphatidylethanolamine (PEG-PE). Such preparations are extruded through filters of defined pore size to yield liposomes with a desired diameter. F_{ab′} fragments of the antibody can be conjugated to the liposomes (Martin and Papahadjopoulos, 1982) via a disulfide-interchange reaction. A chemotherapeutic agent, such as Doxorubicin, may also be contained in the liposome (Gabizon et al., 1989). Other useful liposomes with different compositions are widely available and are contemplated.

Diagnostic Applications of Abs Directed Against GPCR-Like RAIG1

Anti-GPCR-like RAIG1 Abs can be used to localize and/or quantitate GPCR-like RAIG1 (e.g., for use in measuring levels of GPCR-like RAIG1 within tissue samples or for use in diagnostic methods, etc.). Anti-GPCR-like RAIG1 epitope Abs can be utilized as pharmacologically active compounds.

Anti-GPCR-like RAIG1 Abs can be used to isolate a specific GPCR-like RAIG1 by standard techniques, such as immunoaffinity chromatography or immunoprecipitation. These approaches facilitate purifying endogenous GPCR-like RAIG1 antigen-containing polypeptides from cells and tissues. Such approaches can be used to detect GPCR-like RAIG1 in a sample to evaluate the abundance and pattern of expression of the antigenic polypeptide. Anti-GPCR-like RAIG1 Abs can be used to monitor polypeptide levels in tissues as part of a clinical testing procedure; for example, to determine the efficacy of a given treatment regimen. Coupling the antibody to a detectable substance (label) allows detection of Ab-antigen complexes. Classes of labels include fluorescent, luminescent, bioluminescent, and radioactive materials, enzymes and prosthetic groups. Useful labels include horseradish peroxidase, alkaline phosphatase, β-galactosidase, acetylcholinesterase, streptavidin/biotin, avidin/biotin, umbelliferone, fluorescein, fluorescein isothiocyanate, rhodamine, dichlorotriazinylamine fluorescein, dansyl chloride, phycoerythrin, luminol, luciferase, luciferin, aequorin, and ¹²⁵I, ¹³¹I, ³⁵S or ³H.

Antibody Therapeutics

Abs can be used therapeutically to treat or prevent a disease or pathology in a subject. An antibody preparation, preferably one having high antigen specificity and affinity, generally mediates an effect by binding the target epitope(s). Administration of such Abs may mediate one of two effects: (1) the antibody may prevent ligand binding, eliminating endogenous ligand binding and subsequent signal transduction, or (2) the antibody elicits a physiological response by binding an effector site on the target molecule, initiating signaling.

A therapeutically effective amount of an Ab relates generally to the amount needed to achieve a therapeutic objective, epitope binding affinity, administration rate, and depletion rate of the Ab from a subject. Common ranges for therapeutically effective doses are about 0.1 mg/kg body weight to about 50 mg/kg body weight. Dosing frequencies may range, for example, from twice daily to once a week.

GPCR-Like RAIG1 Recombinant Expression Vectors and Host Cells

Vectors are tools used to shuttle DNA between host cells or as a means to express a polynucleotide sequence. Some vectors function only in prokaryotes, while others function in both prokaryotes and eukaryotes, enabling large-scale DNA preparation from prokaryotes for expression in eukaryotes. Inserting the DNA of interest, such as a GPCR-like RAIG1 sequence or a fragment, is accomplished by ligation techniques and/or mating protocols well known to the skilled artisan. Such DNA is inserted such that its integration does not disrupt any necessary components of the vector. In the case of vectors that are used to express the inserted DNA as a polypeptide, the introduced DNA is operably-linked to the vector elements that govern its transcription and translation.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements, such as promoters, that signal to the host cell to transcribe the inserted DNA. Some promoters are exceptionally useful, such as inducible promoters that control gene transcription in response to specific factors. Operably-linking a GPCR-like RAIG1 or anti-sense construct to an inducible promoter can control the expression of a GPCR-like RAIG1, fragments, or anti-sense constructs. Examples of inducible promoters include those that are tissue-specific, which relegate expression to certain cell types, steroid-responsive (e.g., glucocorticoids (Kaufman, 1990) and tetracycline), or heat-shock reactive. Some bacterial repression systems, such as the lac operon, have been exploited in mammalian cells and transgenic animals (Fieck et al., 1992; Wyborski et al., 1996; Wyborski and Short, 1991). Other desirable inducible promoters include those that are not endogenous to the cells in which the construct is being introduced, but, however, are responsive in those cells when the induction agent is exogenously supplied.

Vectors have many manifestations. A “plasmid” is a circular double stranded DNA molecule that can accept additional DNA fragments. Viral vectors can also accept additional DNA segments into the viral genome. Certain vectors are capable of autonomous replication in a host cell (e.g., bacterial vectors having a bacterial origin of replication and episomal mammalian vectors). Other vectors (e.g., non-episomal mammalian vectors) integrate into the genome of a host cell and replicate as part of the host genome. In general, useful expression vectors are plasmids and viral vectors (e.g., replication defective retroviruses, adenoviruses and adeno-associated viruses); other expression vectors can also be used.

Recombinant expression vectors that comprise a GPCR-like RAIG1 (or fragment(s)) regulate a GPCR-like RAIG1 transcription by exploiting one or more host cell-responsive (or that can be manipulated in vitro) regulatory sequences that is operably-linked to GPCR-like RAIG1.

Vectors can be introduced in a variety of organisms and cells (Table D). Alternatively, the vectors can be transcribed and translated in vitro, for example, using T7 promoter regulatory sequences and T7 polymerase.

TABLE D
Examples of hosts for cloning or expression

| Organisms | Examples | Sources and References* |
|---|---|---|
| Prokaryotes | | |
| Enterobacteriaceae | E. coli | |
| | K 12 strain MM294 | ATCC 31,446 |
| | X1776 | ATCC 31,537 |
| | W3110 | ATCC 27,325 |
| | K5 772 | ATCC 53,635 |
| | Enterobacter | |
| | Erwinia | |
| | Klebsiella | |
| | Proteus | |
| | Salmonella (S. tyhpimurium) | |
| | Serratia (S. marcescans) | |
| | Shigella | |
| | Bacilli (B. subtilis and B. licheniformis) | |
| | Pseudomonas (P. aeruginosa) | |
| | Streptomyces | |
| Eukaryotes | | |
| Yeasts | Saccharomyces cerevisiae | |
| | Schizosaccharomyces | |
| | pombe | |
| | Kluyveromyces | (Fleer et al., 1991) |
| | K. lactis MW98-8C, | (de Louvencourt et al., 1983) |
| | CBS683, CBS4574 | |
| | K. fragilis | ATCC 12,424 |
| | K. bulgaricus | ATCC 16,045 |
| | K. wickeramii | ATCC 24,178 |
| | K. waltii | ATCC 56,500 |
| | K. drosophilarum | ATCC 36,906 |
| | K. thermotolerans | |
| | K. marxianus; yarrowia | (EPO 402226, 1990) |
| | Pichia pastoris | (Sreekrishna et al., 1988) |
| | Candida | |
| | Trichoderma reesia | |
| | Neurospora crassa | (Case et al., 1979) |
| | Torulopsis | |
| | Rhodotorula | |
| | Schwanniomyces (S. occidentalis) | |
| Filamentous Fungi | Neurospora | |
| | Penicillium | |
| | Tolypocladium | (WO 91/00357, 1991) |
| | Aspergillus (A. nidulans | (Kelly and Hynes, 1985; |
| | and A. niger) | Tilburn et al., 1983; Yelton et |
| | | al., 1984) |
| Invertebrate cells | Drosophila S2 | |
| | Spodoptera Sf9 | |
| Vertebrate cells | Chinese Hamster Ovary | |
| | (CHO) | |
| | simian COS | ATCC CRL 1651 |
| | COS-7 | |
| | HEK 293 | |

*Unreferenced cells are generally available from American Type Culture Collection (ATCC; Manassas, VA).

Vector choice is dictated by the organisms or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. Vectors often use a selectable marker to facilitate identifying those cells that have incorporated the vector. Many selectable markers are well known in the art for the use with prokaryotes, usually antibiotic-resistance genes or the use of autotrophy and auxotrophy mutants. Table F summarizes many of the available markers.

“Host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art (see examples in Table E). The choice of host cell dictates the preferred technique for introducing the polynucleotide of interest. Introduction of polynucleotides into an organism may also be done with ex vivo techniques that use an in vitro method of transfection, as well as established genetic techniques, if any, for that particular organisms.

TABLE E
Methods to introduce polynucleotide into cells

| Cells | Methods | References | Notes |
|---|---|---|---|
| Prokaryotes | Calcium chloride | (Cohen et al., 1972; Hanahan, 1983; Mandel and | |
| (bacteria) | | Higa, 1970) | |
| | Electroporation | (Shigekawa and Dower, 1988) | |
| Eukaryotes | Calcium phosphate | N-(2-Hydroxyethyl)piperazine-N′-(2- | Cells may be “shocked” with |
| Mammalian cells | transfection | ethanesulfonic acid (HEPES) buffered saline | glycerol or dimethylsulfoxide |
| | | solution (Chen and Okayama, 1988; Graham and | (DMSO) to increase transfection |
| | | van der Eb, 1973; Wigler et al., 1978) | efficiency (Ausubel et al., 1987). |
| | | BES (N,N-bis(2-hydroxyethyl)-2- | |
| | | aminoethanesulfonic acid) buffered solution | |
| | | (Ishiura et al., 1982) | |
| | Diethylaminoethyl | (Fujita et al., 1986; Lopata et al., 1984; Selden et | Most useful for transient, but not |
| | (DEAE)-Dextran | al., 1986) | stable, transfections. |
| | transfection | | Chloroquine can be used to |
| | | | increase efficiency. |
| | Electroporation | (Neumann et al., 1982; Potter, 1988; Potter et | Especially useful for hard-to- |
| | | al., 1984; Wong and Neumann, 1982) | transfect lymphocytes. |
| | Cationic lipid reagent | (Elroy-Stein and Moss, 1990; Felgner et al., | Applicable to both in vivo and in |
| | transfection | 1987; Rose et al., 1991; Whitt et al., 1990) | vitro transfection. |
| | Retroviral | Production exemplified by (Cepko et al., 1984; | Lengthy process, many packaging |
| | | Miller and Buttimore, 1986; Pear et al., 1993) | lines available at ATCC. |
| | | Infection in vitro and in vivo: (Austin and | Applicable to both in vivo and in |
| | | Cepko, 1990; Bodine et al., 1991; Fekete and | vitro transfection. |
| | | Cepko, 1993; Lemischka et al., 1986; Turner et | |
| | | al., 1990; Williams et al., 1984) | |
| | Polybrene | (Chaney et al., 1986; Kawai and Nishizawa, | |
| | | 1984) | |
| | Microinjection | (Capecchi, 1980) | Can be used to establish cell lines |
| | | | carrying integrated copies of DFF |
| | | | DNA sequences. |
| | Protoplast fusion | (Rassoulzadegan et al., 1982; Sandri-Goldin et | |
| | | al., 1981; Schaffner, 1980) | |
| Insect cells (in | Baculovirus systems | (Luckow, 1991; Miller, 1988; O′Reilly et al., | Useful for in vitro production of |
| vitro) | | 1992) | polypeptides with eukaryotic |
| | | | modifications. |
| Yeast | Electroporation | (Becker and Guarente, 1991) | |
| | Lithium acetate | (Gietz et al., 1998; Ito et al., 1983) | |
| | Spheroplast fusion | (Beggs, 1978; Hinnen et al., 1978) | Laborious, can produce |
| | | | aneuploids. |
| Plant cells | Agrobacterium | (Bechtold and Pelletier, 1998; Escudero and | |
| (general | transformation | Hohn, 1997; Hansen and Chilton, 1999; Touraev | |
| reference: | | and al., 1997) | |
| (Hansen and | | | |
| Wright, 1999)) | | | |
| | Biolistics | (Finer et al., 1999; Hansen and Chilton, 1999; | |
| | (microprojectiles) | Shillito, 1999) | |
| | Electroporation | (Fromm et al., 1985; Ou-Lee et al., 1986; | |
| | (protoplasts) | Rhodes et al., 1988; Saunders et al., 1989) | |
| | | May be combined with liposomes (Trick and al., | |
| | | 1997) | |
| | Polyethylene glycol | (Shillito, 1999) | |
| | (PEG) treatment | | |
| | Liposomes | May be combined with electroporation (Trick | |
| | | and al., 1997) | |
| | in planta | (Leduc and al., 1996; Zhou and al., 1983) | |
| | microinjection | | |
| | Seed imbibition | (Trick and al., 1997) | |
| | Laser beam | (Hoffman, 1996) | |
| | Silicon carbide | (Thompson and al., 1995) | |
| | whiskers | | |

TABLE F
Useful selectable markers for eukaryote cell transfection

| Selectable Marker | Selection | Action | Reference |
|---|---|---|---|
| Adenosine deaminase (ADA) | Media includes 9-β-D- | Conversion of Xyl-A to Xyl-ATP, which | (Kaufman et al., |
| | xylofuranosyl adenine (Xyl-A) | incorporates into polynucleotides, killing | 1986) |
| | | cells. ADA detoxifies | |
| Dihydrofolate reductase | Methotrexate (MTX) and | MTX competitive inhibitor of DHFR. In | (Simonsen and |
| (DHFR) | dialyzed serum (purine-free | absence of exogenous purines, cells require | Levinson, |
| | media) | DHFR, a necessary enzyme in purine | 1983) |
| | | biosynthesis. | |
| Aminoglycoside | G418 | G418, an aminoglycoside detoxified by APH, | (Southern and |
| phosphotransferase (“APH”, | | interferes with ribosomal function and | Berg, 1982) |
| “neo”, “G418”) | | consequently, translation. | |
| Hygromycin-B- | hygromycin-B | Hygromycin-B, an aminocyclitol detoxified | (Palmer et al., |
| phosphotransferase (HPH) | | by HPH, disrupts polypeptide translocation | 1987) |
| | | and promotes mistranslation. | |
| Thymidine kinase (TK) | Forward selection (TK+): Media | Forward: Aminopterin forces cells to | (Littlefield, |
| | (HAT) incorporates aminopterin. | synthesze dTTP from thymidine, a pathway | 1964) |
| | Reverse selection (TK−): Media | requiring TK. | |
| | incorporates 5- | Reverse: TK phosphorylates BrdU, which | |
| | bromodeoxyuridine (BrdU). | incorporates into polynucleotides, killing | |
| | | cells. | |

A host cell, prokaryotic or eukaryotic, can be used to produce GPCR-like RAIG1 in culture. To accomplish in vitro expression of GPCR-like RAIG1, a host cell containing a recombinant expression vector encoding GPCR-like RAIG1 is expressed when cultured in a suitable medium. The GPCR-like RAIG1 may then be isolated from the media or culture.

Transgenic GPCR-Like RAIG1 Animals

Transgenic animals are useful for studying the function and/or activity of a GPCR-like RAIG1 and for identifying and/or evaluating modulators of a GPCR-like RAIG1 activity. “Transgenic animals” are non-human animals, preferably mammals, more preferably rodents such as rats or mice, in which one or more of the cells include a transgene. Other transgenic animals include primates, sheep, dogs, cows, goats, chickens, amphibians, etc. A “transgene” is exogenous DNA that is integrated into the genome of a cell from which a transgenic animal develops and that remains in the genome of the mature animal. Transgenes preferably direct the expression of an encoded gene product in one or more cell types or tissues, preventing expression of a naturally encoded gene product in one or more cell types or tissues (a “knockout” transgenic animal), over-expressing an encoded gene, or serving as a marker or indicator of an integration, chromosomal location, or region of recombination (e.g. cre/loxP mice). A “homologous recombinant animal” is a non-human animal, such as a rodent, in which an endogenous GPCR-like RAIG1 has been altered by an exogenous DNA molecule that recombines homologously with an endogenous GPCR-like RAIG1 in a (e.g. embryonic) cell prior to development of the animal. Host cells with an exogenous GPCR-like RAIG can be used to produce non-human transgenic animals, such as fertilized oocytes or embryonic stem cells into which a GPCR-like RAIG1 coding sequence has been introduced. Such host cells can then be used to create non-human transgenic animals or homologous recombinant animals.

Approaches to Transgenic Animal Production

A transgenic animal can be created by introducing a GPCR-like RAIG1 into the male pronuclei of a fertilized oocyte (e.g., by microinjection, retroviral infection, etc.) and allowing the oocyte to develop in a pseudopregnant female foster animal (pffa). The GPCR-like RAIG1 sequence (SEQ ID NO:1) or homolog can be introduced as a transgene into the genome of a non-human animal. Intronic sequences and polyadenylation signals can also be included in the transgene to increase transgene expression. Tissue-specific regulatory sequences can be operably-linked to the GPCR-like RAIG1 transgene to direct expression of GPCR-like RAIG1 to particular cells. Methods for generating transgenic animals via embryo manipulation and microinjection, particularly animals such as mice, have become conventional in the art, (e.g., Evans et al., U.S. Pat. No. 4,870,009, 1989; Hogan, 0879693843, 1994; Leder and Stewart, U.S. Pat. No. 4,736,866, 1988; Wagner and Hoppe, U.S. Pat. No. 4,873,191, 1989). Other non-mice transgenic animals may be made by similar methods. A transgenic founder animal, which can be used to breed additional transgenic animals, can be identified based upon the presence of the transgene in its genome and/or transgene mRNA expression in tissues or cells of the animals. Transgenic (e.g. GPCR-like RAIG1) animals can be bred to other transgenic animals carrying other transgenes.

Vectors for Transgenic Animal Production

To create a homologous recombinant animal, a vector containing at least a portion of GPCR-like RAIG1 into which a deletion, addition or substitution has been introduced to thereby alter, e.g., functionally disrupt, the GPCR-like RAIG1. The GPCR-like RAIG1 can be a mouse gene (SEQ ID NO:1), or a GPCR-like RAIG1 homolog. In one approach, a knockout vector functionally disrupts an endogenous GPCR-like RAIG1 gene upon homologous recombination, and thus a non-functional GPCR-like RAIG1, if any, is expressed.

Alternatively, the vector can be designed such that, upon homologous recombination, an endogenous GPCR-like RAIG1 is mutated or otherwise altered but still encodes functional polypeptide (e.g., the upstream regulatory region can be altered to thereby alter the expression of an endogenous GPCR-like RAIG1). In this type of homologous recombination vector, the altered portion of a GPCR-like RAIG1 is flanked at its 5′- and 3′-termini by additional polynucleotides of a GPCR-like RAIG1 to allow for homologous recombination to occur between the exogenous GPCR-like RAIG1 carried by the vector and an endogenous GPCR-like RAIG1 in an embryonic stem cell. The additional flanking GPCR-like RAIG1 polynucleotide is sufficient to engender homologous recombination with the target endogenous GPCR-like RAIG1. Typically, several kilobases of flanking DNA (both at the 5′- and 3′-termini) are included in the vector (Thomas and Capecchi, 1987). The vector is then introduced into an embryonic stem cell line, and cells in which the introduced GPCR-like RAIG1 has homologously-recombined with an endogenous GPCR-like RAIG1 are selected (Li et al., 1992).

Introduction of GPCR-Like RAIG1 Transgene Cells During Development

Selected cells are then injected into a blastocyst of an animal to form aggregation chimeras (Bradley, 1987). A chimeric embryo can then be implanted into a suitable pffa and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are well-described (Berns et al., WO 93/04169, 1993; Bradley, 1991; Kucherlapati et al., WO 91/01140, 1991; Le Mouellic and Brullet, WO 90/11354, 1990).

Alternatively, transgenic animals that contain selected systems that allow for regulated expression of the transgene can be produced. For example, the cre/loxP recombinase system of bacteriophage P1 (Lakso et al., 1992) or the FLP recombinase system of Saccharomyces cerevisiae (O'Gorman et al., 1991) may be used. In cre/loxP recombinase systems, animals containing transgenes encoding both the Cre recombinase and a selected polypeptide are required. Such animals can be produced as “double” transgenic animals, by mating an animal containing a transgene encoding a selected polypeptide to another containing a transgene encoding a recombinase.

Clones of transgenic animals can also be produced (Wilmut et al., 1997). In brief, a cell from a transgenic animal can be isolated and induced to exit the growth cycle and enter G₀ phase. The quiescent cell can then be fused to an enucleated oocyte from an animal of the same species from which the quiescent cell is isolated. The reconstructed oocyte is then cultured to develop to a morula or blastocyte and then transferred to a pffa. The offspring borne of this female foster animal will be a clone of the “parent” transgenic animal.

Pharmaceutical Compositions

The GPCR-like RAIG1 and GPCR-like RAIG1 molecules, and anti-GPCR-like RAIG1 Abs, their derivatives, fragments, analogs and homologs, can be incorporated into pharmaceutical compositions. Such compositions typically also comprise a pharmaceutically acceptable carrier. A “pharmaceutically acceptable carrier” includes any and all solvents, dispersion media, coatings, antibacterial and antifungal agents, isotonic and absorption delaying agents and the like that are compatible with pharmaceutical administration (Gennaro, 2000). Preferred examples of such carriers or diluents include, but are not limited to, water, saline, Ringer's solution, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

General Considerations

A pharmaceutical composition is formulated to be compatible with the intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous applications include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

Injectable Formulations

To access adipose tissue, injection provides a direct and facile route, especially for that tissue that is below the skin. Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, such as parabens, chlorobutanol, phenol, ascorbic acid, and thimerosal, can control microorganism contamination. Isotonic agents, such as sugars, polyalcohols such as manitol, sorbitol, and sodium chloride can be included in the composition. Compositions that delay absorption include agents such as aluminum monostearate and gelatin.

Sterile injectable solutions can be prepared by incorporating the active compound (e.g., GPCR-like RAIG1 or anti-GPCR-like RAIG1 antibody) in an appropriate solvent with one or a combination of ingredients, followed by sterilization. Generally, dispersions are prepared by incorporating the active compound into a sterile vehicle that contains a basic dispersion medium, and any other required ingredients. Sterile powders for the preparation of sterile injectable solutions methods of preparation include vacuum drying and freeze-drying that yield a powder containing the active ingredient and any desired ingredient from a sterile solutions.

Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

Compositions for Inhalation

For administration by inhalation, the compounds are delivered as an aerosol spray from a nebulizer or a pressurized container that contains a suitable propellant, e.g., a gas such as carbon dioxide.

Systemic Administration

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams. The compounds can also be prepared in the form of suppositories (e.g., with bases such as cocoa butter and other glycerides) or retention enemas for rectal delivery.

Carriers

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid (ALZA Corporation; Mountain View, Calif. and NOVA Pharmaceuticals, Inc.; Lake Elsinore, Calif.; or prepared by one of skill in the art). Liposomal suspensions can also be used as pharmaceutically acceptable carriers. These can be prepared according to known methods (Eppstein et al., U.S. Pat. No. 4,522,811, 1985).

Unit Dosage

Oral formulations or parenteral compositions in unit dosage form can be created to facilitate administration and dosage uniformity. Unit dosage form refers to physically discrete units suited as single dosages for a subject to be treated, containing a therapeutically effective quantity of active compound in association with the required pharmaceutical carrier. The specification for unit dosage forms are dictated by, and directly dependent on, the unique characteristics of the active compound and the particular desired therapeutic effect, and the inherent limitations of compounding the active compound

Gene Therapy Compositions

The polynucleotide molecules of the invention can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Dosage

The pharmaceutical compositions and methods of the present invention may further comprise other therapeutically active compounds that are usually applied in the treatment of adipose-related pathologies.

In the treatment or prevention of conditions which require modulation of GPCR-like RAIG1, an appropriate dosage level will generally be about 0.01 to 500 mg per kg patient body weight per day which can be administered in single or multiple doses. Preferably, the dosage level will be about 0.1 to about 250 mg/kg per day, more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1 to 1000 mg of the active ingredient, particularly 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to a patient to be treated. The compounds may be administered on a regimen of one to four times per day, preferably once or twice per day.

It will be understood, however, that the specific dose level and frequency of dosage for any particular patient may be varied and depends upon a variety of factors, including the activity of the specific compound employed, the metabolic stability and length of action of that compound, the age, body weight, general health, sex, diet, mode and time of administration, rate of excretion, drug combination, the severity of the particular condition, and the host undergoing therapy.

Kits for Pharmaceutical Compositions

The pharmaceutical compositions can be included in a kit, container, pack, or dispenser together with instructions for administration. When the invention is supplied as a kit, the different components of the composition may be packaged in separate containers and admixed immediately before use. Such separate packaging of the components permits long-term storage without losing the active components' functions.

Kits may also include reagents in separate containers that facilitate the execution of a specific test, such as diagnostic tests or tissue typing. For example, GPCR-like RAIG1 DNA templates and suitable primers may be supplied for internal controls.

(a) Containers or Vessels

The reagents included in the kits can be supplied in containers of any sort such that the life of the different components are preserved and are not adsorbed or altered by the materials of the container. For example, sealed glass ampules may contain lyophilized GPCR-like RAIG1 or buffer that has been packaged under a neutral non-reacting gas, such as nitrogen. Ampules may consist of any suitable material, such as glass; organic polymers, such as polycarbonate, polystyrene, etc.; ceramic, metal or any other material typically employed to hold reagents. Other examples of suitable containers include simple bottles that may be fabricated from similar substances as ampules, and envelopes that may have foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail.

Screening and Detection Methods

Isolated GPCR-like RAIG1 polynucleotide molecules of the invention can be used to express GPCR-like RAIG1 (e.g., via a recombinant expression vector in a host cell in gene therapy applications), to detect GPCR-like RAIG1 mRNA (e.g., in a biological sample) or a genetic lesion in GPCR-like RAIG1, and to modulate GPCR-like RAIG1 activity. In addition, GPCR-like RAIG1 polypeptides can be used to screen drugs or compounds that modulate GPCR-like RAIG1 activity or expression as well as to treat disorders characterized by insufficient or excessive production of a GPCR-like RAIG1 or production of forms of GPCR-like RAIG1 that have decreased or aberrant activity compared to GPCR-like RAIG1 wild-type polypeptide, or modulate biological function that involve GPCR-like RAIG1. In addition, the anti-GPCR-like RAIG1 Abs of the invention can be used to detect and isolate GPCR-like RAIG1 and modulate GPCR-like RAIG1 activity.

Screening Assays

The invention provides a method (screening assay) for identifying modalities, i.e., candidate or test compounds or agents (e.g., peptides, peptidomimetics, small molecules or other drugs), foods, combinations thereof, etc., that effect GPCR-like RAIG1 as a stimulatory or inhibitory effect, including translation, transcription, activity or copies of the gene in cells. The invention also includes compounds identified in screening assays.

Testing for compounds that increase or decrease GPCR-like RAIG1 activity are desirable. A compound may modulate GPCR-like RAIG1 activity by affecting: (1) the number of copies of the gene in the cell (amplifiers and deamplifiers); (2) increasing or decreasing transcription of the GPCR-like RAIG1 (transcriptional up-regulators and down-regulators); (3) by increasing or decreasing translation of GPCR-like RAIG1 mRNA (translational up-regulators and down-regulators); or (4) by increasing or decreasing the activity of GPCR-like RAIG1 itself (agonists and antagonists).

(a) Effects of Compounds

To identify compounds that affect GPCR-like RAIG1 at the DNA, RNA and polypeptide levels, cells or organisms are contacted with a candidate compound, and the corresponding change in the target GPCR-like RAIG1 DNA, RNA or polypeptide is assessed (Ausubel et al., 1990). For DNA amplifiers and deamplifiers, the amount of GPCR-like RAIG1 DNA is measured; for those compounds that are transcription up-regulators and down-regulators, the amount of GPCR-like RAIG1 mRNA is determined; for translational up- and down-regulators, the amount of GPCR-like RAIG1 polypeptides is measured. Compounds that are agonists or antagonists may be identified by contacting cells or organisms with the compound.

Many assays for screening candidate or test compounds that bind to or modulate the activity of a GPCR-like RAIG1 or GPCR-like RAIG1 or biologically active portions are available. Test compounds can be obtained using any of the numerous approaches in combinatorial library methods, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptides, while the other four approaches encompass peptide, non-peptide oligomer or small molecule libraries of compounds (Lam, 1997).

(b) Small Molecules

A “small molecule” refers to a composition that has a molecular weight of less than about 5 kD and more preferably less than about 4 kD, and most preferably less than 0.6 kD. Small molecules can be polynucleotides, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. Examples of methods for the synthesis of molecular libraries have been well described (Carell et al., 1994a; Carell et al., 1994b; Cho et al., 1993; DeWitt et al., 1993; Gallop et al., 1994; Zuckermann et al., 1994).

Libraries of compounds may be presented in solution (Houghten et al., 1992) on beads (Lam et al., 1991), on chips (Fodor et al., 1993), bacteria, spores (Ladner et al., U.S. Pat. No. 5,223,409, 1993), plasmids (Cull et al., 1992) or phage (Cwirla et al., 1990; Devlin et al., 1990; Felici et al., 1991; Ladner et al., U.S. Pat. No. 5,223,409, 1993; Scott and Smith, 1990). A cell-free assay comprises contacting a GPCR-like RAIG1 or biologically-active fragment with a known compound that binds GPCR-like RAIG1 to form an assay mixture, contacting the assay mixture with a test compound, and determining the ability of the test compound to interact with the target GPCR-like RAIG1, where determining the ability of the test compound to interact with the target GPCR-like RAIG1 comprises determining the ability of the target GPCR-like RAIG1 to preferentially bind to or modulate the activity of a GPCR-like RAIG1 target molecule.

(c) Cell-Free Assays

Cell-free assays may be used with both soluble or membrane-bound forms of the various GPCR-like RAIG1. In the case of cell-free assays comprising membrane-bound forms, a solubilizing agent can be used to maintain GPCR-like RAIG1 in solution. Examples of such solubilizing agents include non-ionic detergents such as n-octylglucoside, n-dodecylglucoside, n-dodecylmalto side, octanoyl-N-methylglucamide, decanoyl-N-methylglucamide, polyoxyethylene ethers, such as t-octylphenoxypolyethoxy ethanol, isotridecypoly(ethylene glycol ether), N-dodecyl-N,N-dimethyl-3-ammonio-1-propane sulfonate, 3-(3-cholamidopropyl)dimethylamminiol-1-propane sulfonate, or 3-(3-cholamidopropyl)dimethylamminiol-2-hydroxy-1-propane sulfonate.

(d) Immobilization of Target Molecules to Facilitate Screening

In more than one embodiment of the assay methods, immobilizing either GPCR-like RAIG1 or one of its partner molecules can facilitate separation of complexed from uncomplexed forms of one or both of the polypeptides, as well as to accommodate high throughput assays. Binding of a test compound to GPCR-like RAIG1, or interaction of GPCR-like RAIG1 with a target molecule in the presence and absence of a candidate compound, can be accomplished in any vessel suitable for containing the reactants, such as microtiter plates, test tubes, and micro-centrifuge tubes. A fusion polypeptide can be provided that adds a domain that allows one or both of the polypeptides to be bound to a matrix. For example, GST-GPCR-like RAIG1 fusion polypeptides or GST-target fusion polypeptides can be adsorbed onto glutathione sepharose beads or glutathione derivatized microtiter plates that are then combined with the test compound or the test compound and either the non-adsorbed target polypeptide or a GPCR-like RAIG1, and the mixture is incubated under conditions conducive to complex formation (e.g., at physiological conditions for salt and pH). Following incubation, the beads or microtiter plate wells are washed to remove any unbound components, the matrix immobilized in the case of beads, and complex formation determined either directly or indirectly. Alternatively, the complexes can be dissociated from the matrix, and the level of GPCR-like RAIG1 binding or activity determined using standard techniques.

Other techniques for immobilizing polypeptides on matrices can also be used in screening assays. Either GPCR-like RAIG1 or its target molecule can be immobilized using biotin-avidin or biotin-streptavidin systems. Biotinylation can be accomplished using many reagents, such as biotin-NHS (N-hydroxy-succinimide; Pierce Chemicals, Rockford, Ill.), and immobilized in wells of streptavidin-coated 96 well plates (Pierce Chemical). Alternatively, Abs reactive with GPCR-like RAIG1 or other target molecules, but which do not interfere with binding of GPCR-like RAIG1 to its target molecule, can be derivatized to the wells of the plate, and unbound target or GPCR-like RAIG1 trapped in the wells by antibody conjugation. Methods for detecting such complexes, in addition to those described for the GST-immobilized complexes, include immunodetection of complexes using Abs reactive with GPCR-like RAIG1 or its target, as well as enzyme-linked assays that rely on detecting an enzymatic activity associated with the GPCR-like RAIG1 or target molecule.

(e) Screens to Identify Modulators

Modulators of the expression of GPCR-like RAIG1 can be identified in a method where a cell is contacted with a candidate compound and the expression of GPCR-like RAIG1 mRNA or polypeptide in the cell is determined. The expression level of GPCR-like RAIG1 mRNA or polypeptide in the presence of the candidate compound is compared to GPCR-like RAIG1 mRNA or polypeptide levels in the absence of the candidate compound. The candidate compound can then be identified as a modulator of GPCR-like RAIG1 mRNA or polypeptide expression based upon this comparison. For example, when expression of GPCR-like RAIG1 mRNA or polypeptide is greater (statistically significant) in the presence of the candidate compound than in its absence, the candidate compound is identified as a stimulator of GPCR-like RAIG1 mRNA or polypeptide expression. Alternatively, when expression of GPCR-like RAIG1 mRNA or polypeptide is less (statistically significant) in the presence of the candidate compound than in its absence, the candidate compound is identified as an inhibitor of GPCR-like RAIG1 mRNA or polypeptide expression. The level of GPCR-like RAIG1 mRNA or polypeptide expression in cells can be determined by methods described for detecting GPCR-like RAIG1 mRNA or polypeptide.

(i) Hybrid Assays

In yet another aspect of the invention, GPCR-like RAIG1s can be used as “bait” in two- or three-hybrid assays (Bartel et al., 1993; Brent et al., WO94/10300, 1994; Iwabuchi et al., 1993; Madura et al., 1993; Saifer et al., U.S. Pat. No. 5,283,317, 1994; Zervos et al., 1993) to identify other polypeptides that bind or interact with GPCR-like RAIG1 and modulate GPCR-like RAIG1 activities. Such GPCR-like RAIG1-binding partners are also likely to be involved in the propagation of signals by GPCR-like RAIG1 as, for example, upstream or downstream elements of a GPCR-like RAIG1 pathway.

The two-hybrid system is based on the modular nature of most transcription factors, which consist of separable DNA-binding and activation domains. Briefly, the assay utilizes two different DNA constructs. In one construct, the gene that codes for GPCR-like RAIG1 is fused to a gene encoding a DNA binding domain of a known transcription factor (e.g., GAL4). The other construct, a DNA sequence from a library of DNA sequences that encodes an unidentified polypeptide (“prey” or “sample”) is fused to a gene that codes for the activation domain of the known transcription factor. If the “bait” and the “prey” polypeptides are able to interact in vivo, forming a GPCR-like RAIG1-dependent complex, the DNA-binding and activation domains of the transcription factor are brought into close proximity. This proximity allows transcription of a reporter gene (e.g., lacZ) that is operably-linked to a transcriptional regulatory site responsive to the transcription factor. Expression of the reporter gene can be detected, and cell colonies containing the functional transcription factor can be isolated and used to obtain the cloned gene that encodes the GPCR-like RAIG1-interacting polypeptide.

The invention further pertains to novel agents identified by the aforementioned screening assays and their uses for treatments as described herein.

Detection Assays

Portions or fragments of GPCR-like RAIG1 cDNA sequences-and the complete GPCR-like RAIG1 gene sequences-are useful in themselves. These sequences can be used to: (1) identify an individual from a minute biological sample (tissue typing); and (2) aid in forensic identification of a biological sample.

GPCR-like RAIG1 sequences can be used to identify individuals from minute biological samples. In this technique, an individual's genomic DNA is digested with one or more restriction enzymes and probed on a Southern blot to yield unique bands. The sequences of the invention are useful as additional DNA markers for “restriction fragment length polymorphisms” (RFLP); (Smulson et al., U.S. Pat. No. 5,272,057, 1993).

Furthermore, GPCR-like RAIG1 sequences can be used to determine the actual base-by-base DNA sequence of targeted portions of an individual's genome. GPCR-like RAIG1 sequences can be used to prepare two PCR primers from the 5′- and 3′-termini of the sequences that can then be used to amplify an the corresponding sequences from an individual's genome and then sequence the amplified fragment.

Panels of corresponding DNA sequences from individuals can provide unique individual identifications as each individual will have a unique set of such DNA sequences due to allelic differences. The sequences of the invention can be used to identify such sequences from individuals and from tissue. The GPCR-like RAIG1 sequences of the invention uniquely represent portions of an individual's genome. Allelic variation occurs to some degree in the coding regions of these sequences, and to a greater degree in the noncoding regions. The allelic variation between individual humans occurs with a frequency of about once every 500 bases. Much of the allelic variation is due to single polynucleotide polymorphisms (SNPs), including RFLPs.

Each GPCR-like RAIG1 sequence can, to some degree, be used as a standard against which DNA from an individual can be compared for identification purposes. Because greater numbers of polymorphisms occur in noncoding regions, fewer sequences are necessary to differentiate individuals. Noncoding sequences can positively identify individuals with a panel of 10 to 1,000 primers that each yield a noncoding amplified sequence of 100 bases. If predicted coding sequences, such as those in SEQ ID NOS:1 or 3 are used, a more appropriate number of primers for positive individual identification would be 500-2,000.

Predictive Medicine

The invention also pertains to the field of predictive medicine in which diagnostic assays, prognostic assays, pharmacogenomics, and clinical trial monitoring are used for prognostic (predictive) purposes to treat an individual prophylactically. Diagnostic assays, using biological samples (e.g., blood, serum, cells, tissue) to determine the presence of GPCR-like RAIG1 polynucleotide (mRNA) and GPCR-like RAIG1 activity can be used to test whether an individual is afflicted with a disease or disorder or is at risk of developing a disorder associated with aberrant GPCR-like RAIG1 expression or activity, including cachexia. The invention also provides for prognostic (or predictive) assays for determining whether an individual is at risk of developing a disorder associated with GPCR-like RAIG1 polynucleotide expression or activity. For example, mutations in a GPCR-like RAIG1 can be assayed in a biological sample. Such assays can be used for prognostic or predictive purpose to prophylactically treat an individual prior to the onset of a disorder characterized by or associated with GPCR-like RAIG1, polynucleotide expression, or biological activity.

Determining a GPCR-like RAIG1 activity or polynucleotide expression in an individual can be exploited to select appropriate therapeutic or prophylactic agents for that individual (pharmacogenomics). Pharmacogenomics allows for the selection of modalities (e.g., drugs, foods) for therapeutic or prophylactic treatment of an individual based on the individual's genotype (e.g., the individual's genotype to determine the individual's ability to respond to a particular agent). Another aspect of the invention pertains to monitoring the influence of modalities (e.g., drugs, foods) on the expression or activity of GPCR-like RAIG1 in clinical trials.

Diagnostic Assays

An exemplary method for detecting the presence or absence of GPCR-like RAIG1 in a biological sample involves obtaining a biological sample from a subject and contacting the biological sample with a compound or an agent capable of detecting GPCR-like RAIG1 or GPCR-like RAIG1 polynucleotide such that the presence of GPCR-like RAIG1 is confirmed in the sample. An agent for detecting GPCR-like RAIG1 message or DNA is a labeled polynucleotide probe that specifically hybridizes the target GPCR-like RAIG1 RNA or genomic DNA. The polynucleotide probe can be, for example, a full-length GPCR-like RAIG1 polynucleotide, such as the polynucleotide of SEQ ID NO:1 or a portion thereof, such as an oligonucleotide of at least 15, 30, 50, 100, 250 or 500 polynucleotides in length and sufficient to specifically hybridize under stringent conditions to GPCR-like RAIG1 mRNA or genomic DNA.

An agent for detecting GPCR-like RAIG1 polypeptide is an Ab capable of binding to GPCR-like RAIG1, preferably an Ab with a detectable label. Abs can be polyclonal, or more preferably, monoclonal. An intact Ab, or a fragment (e.g., F_{ab} or F(ab′)₂) can be used. A labeled probe or Ab is coupled (i.e., physically linking) to a detectable substance, as well as indirect detection of the probe or Ab by reactivity with another reagent that is directly labeled. Examples of indirect labeling include detection of a primary Ab using a fluorescently labeled secondary Ab and end-labeling of a DNA probe with biotin such that it can be detected with fluorescently-labeled streptavidin. The term “biological sample” includes tissues, cells and biological fluids isolated from a subject, as well as tissues, cells and fluids present within a subject. Biological samples from a subject may contain polypeptide molecules, mRNA molecules and genomic DNA molecules. A preferred biological sample is blood. Detection methods can be used to detect GPCR-like RAIG1 mRNA, polypeptide, or genomic DNA in a biological sample in vitro as well as in vivo. For example, in vitro techniques for detection of GPCR-like RAIG1 mRNA include Northern and in situ hybridizations. In vitro techniques for detection of GPCR-like RAIG1 polypeptide include enzyme-linked immunosorbent assays (ELISAs), Western blots, immunoprecipitations and immunofluorescence. In vitro techniques for detection of GPCR-like RAIG1 genomic DNA include Southern hybridizations and fluorescent in situ hybridization (FISH). Furthermore, in vivo techniques for detecting GPCR-like RAIG1 include introducing into a subject a labeled anti-GPCR-like RAIG1 antibody. For example, the antibody can be labeled with a radioactive marker whose presence and location in a subject can be detected by standard imaging techniques.

The methods further involve obtaining a biological sample from a subject to provide a control, contacting the sample with a compound or agent to detect GPCR-like RAIG1, and comparing the presence of GPCR-like RAIG1 in the control sample with the presence of GPCR-like RAIG1, mRNA or genomic DNA in the test sample. Kits for detecting GPCR-like RAIG1 in a biological sample may also be used.

Prognostic Assays

Diagnostic methods can furthermore be used to identify subjects having, or at risk of developing, a disease or disorder associated with aberrant GPCR-like RAIG1 expression or activity, such as obesity or obesity-related complications. Prognostic assays can be used to identify a subject having or at risk for developing a disease or disorder. A method for identifying a disease or disorder associated with aberrant GPCR-like RAIG1 expression or activity would include a test sample obtained from a subject and detecting a GPCR-like RAIG1 or polynucleotide (e.g., mRNA, genomic DNA). A test sample is a biological sample obtained from a subject. For example, a test sample can be a biological fluid (e.g., serum), cell sample, or tissue.

Prognostic assays can also be used to determine whether a subject can be administered a modality (e.g., an agonist, antagonist, peptidomimetic, polypeptide, peptide, polynucleotide, small molecule, food, etc.) to treat a disease or disorder associated with aberrant GPCR-like RAIG1 expression or activity, such as obesity. Methods for determining whether a subject can be effectively treated with an agent include obtaining a test sample and detecting GPCR-like RAIG1 or polynucleotide (e.g., wherein the presence of GPCR-like RAIG1 or polynucleotide is diagnostic for a subject that can be administered the agent to treat a disorder associated with aberrant GPCR-like RAIG1 expression or activity).

Genetic lesions in GPCR-like RAIG1 can be used to determine if a subject is at risk for a disorder, such as obesity. Methods include detecting in a sample from a subject, the presence or absence of a genetic lesion characterized by at an alteration affecting the integrity of a gene encoding GPCR-like RAIG1 polypeptide or the mis-expression of GPCR-like RAIG1. Such genetic lesions can be detected by ascertaining: (1) a deletion of one or more polynucleotides from GPCR-like RAIG1; (2) an addition of one or more polynucleotides to GPCR-like RAIG1; (3) a substitution of one or more polynucleotides in GPCR-like RAIG1, (4) a chromosomal rearrangement of a GPCR-like RAIG1 gene; (5) an alteration in the level of GPCR-like RAIG1 mRNA transcripts, (6) aberrant modification of GPCR-like RAIG1, such as a change in genomic DNA methylation, (7) the presence of a non-wild-type splicing pattern of a GPCR-like RAIG1 mRNA transcript, (8) a non-wild-type level of GPCR-like RAIG1, (9) allelic loss of GPCR-like RAIG1, and/or (10) inappropriate post-translational modification of GPCR-like RAIG1 polypeptide. There are a large number of known assay techniques that can be used to detect lesions in GPCR-like RAIG1. Any biological sample containing nucleated cells may be used.

Lesion detection may use a probe or primer in a polymerase chain reaction (PCR) (e.g., Mullis, U.S. Pat. No. 4,683,202, 1987; Mullis et al., U.S. Pat. No. 4,683,195, 1987), such as anchor PCR or rapid amplification of cDNA ends (RACE) PCR, or, alternatively, in a ligation chain reaction (LCR) (e.g., (Landegren et al., 1988; Nakazawa et al., 1994), the latter is particularly useful for detecting point mutations in GPCR-like RAIG1 (Abravaya et al., 1995). This method includes collecting a sample from a patient, isolating polynucleotides from the sample (if necessary), contacting the polynucleotides with one or more primers that specifically hybridize to GPCR-like RAIG1 under conditions such that hybridization and amplification of any present GPCR-like RAIG1 occurs, and detecting the presence or absence of an amplification product, or detecting the size of the amplification product and comparing the length to a control sample. PCR and LCR are often desirable to use as a preliminary amplification step in conjunction with any of the techniques used for detecting mutations.

Alternative amplification methods include self sustained sequence replication (Guatelli et al., 1990), transcriptional amplification system (Kwoh et al., 1989); Qβ Replicase (Lizardi et al., 1988), or any other polynucleotide amplification method, followed by the detection of the amplified molecules using techniques well known to those of skill in the art. These detection schemes are especially useful for the detection of low abundant polynucleotide molecules.

Mutations in GPCR-like RAIG1 from a sample can be identified by alterations in restriction enzyme cleavage patterns. For example, sample and control DNA is isolated, amplified if desired, digested with one or more restriction endonucleases, and fragment length sizes are determined by gel electrophoresis and compared. Differences in fragment length sizes between sample and control DNA indicate mutations in the sample DNA. Moreover, the use of sequence specific ribozymes can be used to score for the presence of specific mutations by gain or loss of a ribozyme cleavage site.

Hybridizing a sample and control polynucleotides, e.g., DNA or RNA, to high-density arrays containing hundreds or thousands of oligonucleotides probes, can also identify genetic mutations in GPCR-like RAIG1 (Cronin et al., 1996; Kozal et al., 1996). For example, genetic mutations in GPCR-like RAIG1 can be identified in two-dimensional arrays containing light-generated DNA probes (Cronin et al., 1996). Briefly, a first hybridization array of probes can be used to scan through long stretches of DNA in a sample and control to identify base changes between the sequences by making linear arrays of sequential overlapping probes. This step allows the identification of point mutations. A second hybridization array follows that allows the characterization of specific mutations by using smaller, specialized probe arrays complementary to all variants or mutations detected. Each mutation array is composed of parallel probe sets, one complementary to the wild-type gene and the other complementary to the mutant gene.

Any of a variety of sequencing reactions known in the art can also be used to directly sequence the target GPCR-like RAIG1 and detect mutations by comparing the sequence of the sample GPCR-like RAIG1-with the corresponding wild-type (control) sequence. Examples of sequencing reactions include those based on classic techniques (Maxam and Gilbert, 1977; Sanger et al., 1977). Any of a variety of automated sequencing procedures can be used when performing diagnostic assays (Naeve et al., 1995) including sequencing by mass spectrometry (Cohen et al., 1996; Griffin and Griffin, 1993; Koster, WO94/16101, 1994).

Other methods for detecting mutations in GPCR-like RAIG1 include those in which protection from cleavage agents is used to detect mismatched bases in RNA/RNA or RNA/DNA duplexes (Myers et al., 1985). In general, the technique of “mismatch cleavage” starts by providing duplexes formed by hybridizing labeled RNA or DNA containing the wild-type GPCR-like RAIG1 sequence with potentially mutant RNA or DNA obtained from a sample. The double-stranded duplexes are treated with an agent that cleaves single-stranded regions of the duplex such as those that arise from base pair mismatches between the control and sample strands. For instance, RNA/DNA duplexes can be treated with RNase and DNA/DNA hybrids treated with S₁ nuclease. In other embodiments, either DNA/DNA or RNA/DNA duplexes can be treated with hydroxylamine or osmium tetroxide and with piperidine in order to digest mismatched regions. The digested material is then separated by size on denaturing polyacrylamide gels to determine the mutation site (Grompe et al., 1989; Saleeba and Cotton, 1993). Control DNA or RNA can be labeled for detection.

Mismatch cleavage reactions may employ one or more polypeptides that recognize mismatched base pairs in double-stranded DNA (DNA mismatch repair) in defined systems for detecting and mapping point mutations in GPCR-like RAIG1 cDNAs obtained from samples of cells. For example, the mutY enzyme of E. coli cleaves A at G/A mismatches and the thymidine DNA glycosylase from HeLa cells cleaves T at G/T mismatches (Hsu et al., 1994). According to an exemplary embodiment, a probe based on a wild-type GPCR-like RAIG1 sequence is hybridized to a cDNA or other DNA product from a test cell(s). The duplex is treated with a DNA mismatch repair enzyme, and the cleavage products, if any, can be detected from electrophoresis protocols or the like (Modrich et al., U.S. Pat. No. 5,459,039, 1995).

Electrophoretic mobility alterations can be used to identify mutations in GPCR-like RAIG1. For example, single strand conformation polymorphisms (SSCPs) may be used to detect differences in electrophoretic mobility between mutant and wild type polynucleotides (Cotton, 1993; Hayashi, 1992; Orita et al., 1989). Single-stranded DNA fragments of sample and control GPCR-like RAIG1 polynucleotides are denatured and then renatured. The secondary structure of single-stranded polynucleotides varies according to sequence; the resulting alteration in electrophoretic mobility allows detection of even a single base change. The DNA fragments may be labeled or detected with labeled probes. Assay sensitivity can be enhanced by using RNA (rather than DNA), in which the secondary structure is more sensitive to a sequence changes. The method may use duplex analysis to separate double stranded duplex molecules on the basis of changes in electrophoretic mobility (Keen et al., 1991).

The migration of mutant or wild-type fragments can be assayed using denaturing gradient gel electrophoresis (DGGE; (Myers et al., 1985). In DGGE, DNA is modified to prevent complete denaturation, for example by adding a GC clamp of approximately 40 bp of high-melting point, GC-rich DNA by PCR. A temperature gradient may also be used in place of a denaturing gradient to identify differences in the mobility of control and sample DNA (Rossiter and Caskey, 1990).

Examples of other techniques for detecting point mutations include selective oligonucleotide hybridization, selective amplification, or selective primer extension. For example, oligonucleotide primers can be prepared in which the known mutation is placed centrally and then hybridized to target DNA under conditions that permit hybridization only if a perfect match is found (Saiki et al., 1986; Saiki et al., 1989). Such allele-specific oligonucleotides are hybridized to PCR-amplified target DNA or a number of different mutations when the oligonucleotides are attached to the hybridizing membrane and hybridized with labeled target DNA.

Alternatively, allele specific amplification technology that depends on selective PCR amplification may be used. Oligonucleotide primers for specific amplifications carry the mutation of interest in the center of the molecule (so that amplification depends on differential hybridization (Gibbs et al., 1989)) or at the extreme 3′-terminus of one primer where, under appropriate conditions, mismatch can prevent or reduce polymerase extension (Prosser, 1993). Novel restriction sites in the region of the mutation may be introduced to create cleavage-based detection (Gasparini et al., 1992). Amplification may also be performed using Taq ligase (Barany, 1991). In such cases, ligation occurs only if there is a perfect match at the 3′-terminus of the 5′ sequence, allowing detection of a known mutation by scoring for amplification.

The described methods may be performed, for example, by using pre-packaged kits comprising at least one probe (nucleotide or antibody) that may be conveniently used in clinical settings to diagnose patients exhibiting symptoms or family history of a disease or illness involving GPCR-like RAIG1. Furthermore, any cell type or tissue in which GPCR-like RAIG1 is expressed may be utilized in prognostic assays.

Pharmacogenomics

Agents or modulators that have a stimulatory or inhibitory effect on GPCR-like RAIG1 activity or expression, as identified in a screening assay, can be administered to individuals to treat prophylactically or therapeutically disorders. In conjunction with such treatment, the pharmacogenomics (i.e., the study of the relationship between a subject's genotype and the subject's response to a foreign modality, such as a food, compound or drug) may be considered. Metabolic differences of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Pharmacogenomics can further be used to determine appropriate dosages and therapeutic regimens. Accordingly, the activity of GPCR-like RAIG1, expression of GPCR-like RAIG1 polynucleotide, or GPCR-like RAIG1 mutation(s) in an individual can be determined to guide the selection of appropriate agent(s) for therapeutic or prophylactic treatment.

Pharmacogenomics deals with clinically significant hereditary variations in the response to modalities due to altered modality disposition and abnormal action in affected persons (Eichelbaum and Evert, 1996; Linder et al., 1997). In general, two pharmacogenetic conditions can be differentiated: (1) genetic conditions transmitted as a single factor altering the interaction of a modality with the body (altered drug action) or (2) genetic conditions transmitted as single factors altering the way the body acts on a modality (altered drug metabolism). These pharmacogenetic conditions can occur either as rare defects or as polynucleotide polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) explains the phenomena of some patients who show exaggerated drug response and/or serious toxicity after taking the standard and safe dose of a drug. These polymorphisms are expressed in two phenotypes in the population, the extensive metabolizer (EM) and poor metabolizer (PM). The prevalence of PM is different among different populations. For example, the CYP2D6 gene is highly polymorphic and several mutations have been identified in PM, which all lead to the absence of functional CYP2D6. Poor metabolizers due to mutant CYP2D6 and CYP2C19 frequently experience exaggerated drug responses and side effects when they receive standard doses. If a metabolite is the active therapeutic moiety, PM shows no therapeutic response, as demonstrated for the analgesic effect of codeine mediated by its CYP2D6-formed metabolite morphine. At the other extreme are the so-called ultra-rapid metabolizers who are unresponsive to standard doses. Recently, the molecular basis of ultra-rapid metabolism has been identified to be due to CYP2D6 gene amplification.

The activity of GPCR-like RAIG1, expression of GPCR-like RAIG1 or of GPCR-like RAIG1 mutations in an individual can be determined to select appropriate agent(s) for therapeutic or prophylactic treatment of the individual. In addition, pharmacogenetic studies can be applied to genotyping polymorphic alleles encoding drug-metabolizing enzymes to identify an individual's drug responsiveness phenotype. This knowledge, when applied to dosing or drug selection, can avoid adverse reactions or therapeutic failure and thus enhance therapeutic or prophylactic efficiency when treating a subject with GPCR-like RAIG1 modulator, such as a modulator identified by one of the described exemplary screening assays.

Monitoring Effects During Clinical Trials

Monitoring the influence of agents (e.g., drugs, compounds) on the expression or activity of GPCR-like RAIG1 can be applied not only in basic drug screening, but also in clinical trials. For example, the effectiveness of an agent determined to increase expression of GPCR-like RAIG1, polypeptide levels, or increase GPCR-like RAIG1 activity in screening assays and can be monitored in clinical trails of subjects exhibiting decreased GPCR-like RAIG1 expression, polypeptide levels, or down-regulated GPCR-like RAIG1 activity. Conversely agents that decrease GPCR-like RAIG1 expression, polypeptide levels, or down-regulate GPCR-like RAIG1 activity, can be tested in subjects with decreased gene expression or polypeptide activity. In clinical trials, the expression or activity of GPCR-like RAIG1 and preferably other genes that have been implicated in, for example, obesity, can be used as markers for a particular cell's responsiveness.

For example, modalities that modulate gene expression or activity (e.g., food, compound, drug or small molecule) can be identified. To study the effect of agents, in a clinical trial, on obesity, cells can be isolated and RNA prepared and analyzed for the levels of expression of GPCR-like RAIG1 and other genes implicated in obesity. The gene expression pattern can be quantified by Northern blot analysis, nuclear run-on or RT-PCR experiments, by measuring the amount of polypeptide, or by measuring the activity level of GPCR-like RAIG1 or other gene products. In this manner, the gene expression pattern itself can serve as a marker, indicating the cellular physiological response to the agent. Accordingly, this response state may be determined before and at various points during treatment of the individual with the agent.

The invention provides methods for monitoring the effectiveness of treatment of a subject with an agent (e.g., an agonist, antagonist, polypeptide, peptide, peptidomimetic, polynucleotide, small molecule, food or other drug candidate identified by the screening assays described herein) having in part the steps of (1) obtaining a pre-administration sample from a subject; (2) detecting the level of expression of GPCR-like RAIG1, mRNA, or genomic DNA in the preadministration sample; (3) obtaining one or more post-administration samples from the subject; (4) detecting the level of expression or activity of the GPCR-like RAIG1, mRNA, or genomic DNA in the post-administration samples; (5) comparing the level of expression or activity of the GPCR-like RAIG1 mRNA, or genomic DNA in the pre-administration sample with GPCR-like RAIG1 mRNA, or genomic DNA in the post administration sample or samples; and (6) altering the administration of the agent to the subject accordingly. For example, increased administration of the agent may be desirable to increase the expression or activity of GPCR-like RAIG1 to higher levels than detected, i.e., to increase the effectiveness of the agent. Alternatively, decreased administration of the agent may be desirable to decrease expression or activity of GPCR-like RAIG1 to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant GPCR-like RAIG1 expression or activity, such as obesity, cachexia, diabetes, etc.

Disease and Disorders

Diseases and disorders that are characterized by altered GPCR-like RAIG1 levels or activity may be treated therapeutically or prophylatically with antagonists or agonists. Useful therapeutics include: (1) GPCR-like RAIG1 polypeptides, or analogs, derivatives, fragments or homologs thereof; (2) Abs to GPCR-like RAIG1 polypeptides; (3) GPCR-like RAIG1 polynucleotides; (4) administration of antisense polynucleotide and dysfunctional or (5) modulators that alter the interaction between GPCR-like RAIG1 and its binding partners.

Increased or decreased levels of GPCR-like RAIG1 molecules can be readily detected by quantifying polypeptide or RNA by obtaining a patient tissue sample (e.g., from biopsy tissue) and assaying in vitro for RNA or polypeptide levels, structure or activity of the expressed polypeptides. Methods include immunoassays (e.g., Western blot analysis, immunoprecipitation followed by sodium dodecyl sulfate (SDS) polyacrylamide gel electrophoresis, immunocytochemistry, etc.) and hybridization assays to detect mRNA expression (e.g., Northern assays, dot blots, in situ hybridization, and the like).

Prophylactic Methods

The invention provides methods for preventing a disease or condition associated with an aberrant GPCR-like RAIG1 expression or activity, in a subject, by administering an agent that modulates expression of GPCR-like RAIG1 or GPCR-like RAIG1 activity. Subjects at risk for a disease that is caused or contributed to by aberrant GPCR-like RAIG1 expression or activity can be identified by, for example, any or a combination of diagnostic or prognostic assays. Administration of a prophylactic agent, prior to symptom manifestation, is characteristic of preventing a disease or disorder. Appropriate agents can be determined based on screening assays.

Therapeutic Methods

Modulating GPCR-like RAIG1 expression or activity can be used therapeutically. The invention provides methods of treating an individual afflicted with a disease or disorder characterized by aberrant expression or activity of GPCR-like RAIG1 polypeptide or polynucleotide. For example, an agent or combination of agents that modulate GPCR-like RAIG1 expression or activity is administered. Alternatively, the method involves administering a GPCR-like RAIG1 or polynucleotide molecule to compensate for reduced or aberrant GPCR-like RAIG1 expression or activity.

Determination of the Biological Effect of the Therapeutic

Suitable in vitro or in vivo assays can be performed to determine the effect of a specific therapeutic and whether its administration is indicated for treatment.

In vitro assays may be performed with representative cells involved in the disorder to determine if a therapeutic exerts a desired effect on specific cell types. To test modalities in vivo and in vitro (by harvesting desired cells) suitable animal model systems including, but not limited to, rats, mice, chicken, cows, monkeys and rabbits can be used.

### EXAMPLES

The following examples are included to demonstrate preferred embodiments of the present invention. It should be appreciated by those of skill in the art that the disclosed techniques represent those discovered by the inventors to function well in the practice of the invention. However, many changes can be made in the specific embodiments and still obtain a like or similar result without departing from the spirit and scope of the invention.

### Example 1

### Fasting/Feeding Experiments

Experimental Design Details:

Four groups of mice. n=3/group

Ad lib fed mice.

2. Mice fasted for 4 hours

3. Mice fasted for 24 hours

4. Mice fasted for 24 hours and then refed ad lib for 24 hours.

5. Mice fasted for 48 hours.

6. Mice fasted for 48 hours and then refed ad lib for 24 hours.

All studies were done in accordance with guidelines set forth by the Institutional Animal Care and Use Committee at Genentech (South San Franscisco, Calif.). Male FVB-N/J mice (Jackson Labs, Bar Harbor, Me.) were received at three weeks of age and housed at two mice/cage until tissue harvest at six weeks of age. All mice were fed rodent chow ad libitum (Chow 5010, Ralston Purina; St. Louis, Mo.) and housed on a 12:12 light/dark cycle at 22° C. Following CO₂-induced euthanasia, stomach tissue was excised, carefully cleaned, and snap-frozen in liquid nitrogen for subsequent RNA preparation.

RNA was prepared and reverse-transcribed from the samples from each treatment group, and subjected to Quantitative Expression Analysis (QEA) (Shimkets, et al., 1999).

### Example 2

### GeneCalling (Shimkets et al., 1999)

RNA Isolation

Total RNA was isolated with Trizol (Life Technologies, Grand Island, N.Y.) using 0.1 volume of bromochloropropane for phase separation (Molecular Research Center, Cincinnati, Ohio), and treated with DNase I (Promega, Madison, Wis.) in the presence of 0.01 M dithiothreitol (DTT) and 1 U/1 RNasin (Promega). Following phenol/chloroform extraction, RNA quality was evaluated by spectrophotometry and formaldehyde agarose gel electrophoresis, and yield was estimated by fluorometry with OliGreen (Molecular Probes, Eugene, Oreg.). Poly-A+ RNA was prepared from 100 g total RNA using oligo(dT) magnetic beads (PerSeptive, Cambridge, Mass.), and quantified with fluorometry.

First-strand cDNA was prepared from 1.0 g of poly(A)+ RNA with 200 pmol oligo(dT)25V (V=A, C or G) using 400 U of Superscript II reverse transcriptase (Invitrogen Life Technologies: Carlsbad, Calif.). Second-strand synthesis was performed at 16° C. for 2 hours after addition of 10 U of E. coli DNA ligase, 40 U of E. coli DNA polymerase, and 3.5 U of E. coli RNase H (all from BRL). T4 DNA polymerase (5 U) was added, incubated for 5 min at 16° C., and then treated with arctic shrimp alkaline phosphatase (5 U; United States Biochemicals, Cleveland, Ohio) at 37° C. for 30 minutes cDNA was purified by phenol/chloroform extraction, and the yield was estimated using fluorometry with PicoGreen (Molecular Probes).

cDNA fragmentation was achieved by digestion in a 50 μl reaction mixture containing 5 U of restriction enzyme (6 base-pair cutters) and 1 ng of double-stranded cDNA. Eighty separate sets of cDNA fragmentation reactions were conducted, each with a different pair of restriction enzymes. These were then ligated to complementary amplification tags with ends compatible to the 5′ and 3′ ends of the fragments at 16° C. for 1 hour in 10 mM ATP, 2.5% PEG, 10 units T4 DNA ligase, and ligase buffer 1. Amplification was then performed after addition of 2 μl 10 mM dNTP, 5 μl 10 TB buffer (500 mM Tris, 160 mM (NH4)₂SO₄, 20 mM MgCl₂, pH 9.15), 0.25 μl Klentaq (Clontech Laboratories, Palo Alto, Calif.): PFU (Stratagene, La Jolla, Calif.) (16:1), 32.75 μl H2O. Amplification was carried out for 20 cycles (30 seconds at 96° C., 1 minute at 57° C., 2 minutes at 72° C.), followed by 10 minutes at 72° C. PCR products were purified using streptavidin beads (CPG, Lincoln Park, N.J.). After washing the beads twice with buffer 1 (3 M NaCl, 10 mM Tris-HCl, 1 mM EDTA, pH 7.5), 20 μl of buffer 1 was mixed with the PCR product for 10 minutes at room temperature, separated with a magnet, and washed once with buffer 2 (10 mM Tris, 1 mM EDTA, pH 8.0). The beads were then dried and resuspended in 3 μl of buffer 3 (80% (vol/vol) formamide, 4 mM EDTA, 5% TAMRA- or ROX-tagged molecular size standards (PE-Applied Biosystems, Foster City, Calif.). Following denaturation (96° C. for 3 minutes), samples were loaded onto 5% polyacrylamide, 6 M urea, 0.5 Tris Borate EDTA ultrathin gels and electrophoresed. PCR products were visualized using the fluorescent FAM label at the 5′ end of one of the PCR primers, which ensures that all detected fragments have been digested by both enzymes.

Gel Interpretation

Electrophoresis data were processed using the Open Genome Initiative (OGI) software. Gel images were first visually checked and tracked. Each lane contained the FAM-labeled products of a single reaction plus a sizing ladder spanning 50 to 500 bp. The ladder peaks provide a correlation between camera frames (collected at 1 Hz) and DNA fragment size in base pairs. After tracking, lanes were extracted and the peaks in the sizing ladder were found. Linear interpolation between the ladder peaks converted the fluorescence traces from frames to base pairs. A final quality control step checked for low signal-to-noise, poor peak resolution, missing ladder peaks, and lane-to-lane bleed. Data that pass all of these criteria were submitted as point-by-point length versus amplitude addresses to an Oracle 8 database.

Difference Identification

For each restriction enzyme pair in each sample set a composite trace was calculated, compiling all the individual sample replicates followed by application of a scaling algorithm for best fit to normalize the traces of the experimental set versus that of the control. The scaled traces are then compared on a point-by-point basis to define areas of amplitude difference that meet the minimum prespecified threshold for a significant difference. Once a region of difference has been identified, the local maximum for the corresponding traces of each set was then determined. The variance of the difference was calculated by the following expression:
σ2_{Δ}(j)=_{λ1}(j)²σ²_{Total}(j:S₁)+_{λ2}(j)²σ²_{Total}(j:S₂)
where λ₁(j) and λ₂(j) represent scaling factors and (j:S) represent scaling factors and (j:S) represents the trace composite values over multiple samples. The probability that the difference is statistically significant is calculated by

$$P ( j ) = 1 - {\int }_{- Δ}^{Δ} ⅆ y ( 1 / √ \{ 2 {πσ}_{Δ}^{2} \} ) exp ( - {y}^{2} / 2 {σ}_{Δ}^{2} )$$

where y is the relative intensity. All difference peaks are stored as unique database addresses in the specified expression difference analysis.

Gene Confirmation by Oligonucleotide Poisoning

Restriction fragments that map in end sequence and length to known rat genes are used as templates for the design of unlabeled oligonucleotide primers. An unlabeled oligonucleotide designed against one end of the restriction fragment is added in excess to the original reaction, and is reamplified for an additional 15 cycles. This reaction is then electrophoresed and compared to a control reaction reamplified without the unlabeled oligonucleotide to evaluate the selective diminution of the peak of interest.

RNA Doping

DNA templates for RNA in vitro transcription were generated by PCR amplification using cloned human cDNAs as templates. PCR primers were complementary to plasmid sequences flanking the cDNA inserts. In addition, the sense primer contained the T7 RNA polymerase consensus sequence, and the antisense primer included a stretch of 25 thymidines for the generation of polyadenylated transcripts. In vitro transcription was performed using the MaxiScript transcription kit (Ambion, Austin, Tex.). The transcripts were poly-A selected on biotin-oligo(dT)25 bound to streptavidin MPG beads (CPG Inc.). The RNA products ranged in size between 1,100 and 2,000 nt. The integrity of the products was monitored by agarose gel electrophoresis, and the concentration determined by fluorometry using RiboGreen dye (Molecular Probes) on a SpectraFluor fluorometer (Tecan, Grundig, Austria). The in vitro transcribed RNAs were mixed at defined ratios with HeLa cell poly-A+ RNA (American Type Culture Collection, Manassas, Va.) and the RNA was converted to cDNA and subjected to GeneCalling chemistry and analysis as described (Shimkets et al., 1999).

### REFERENCES

U.S. Pat. No. 4,166,452. Apparatus for testing human responses to stimuli. 1979.
U.S. Pat. No. 4,485,045. Synthetic phosphatidyl cholines useful in forming liposomes. 1984.
U.S. Pat. No. 4,544,545. Liposomes containing modified cholesterol for organ targeting. 1985.
U.S. Pat. No. 4,676,980. Target specific cross-linked heteroantibodies. 1987.
U.S. Pat. No. 4,816,567. Recombinant immunoglobin preparations. 1989.
WO 90/10448. Covalent conjugates of lipid and oligonucleotide. 1990.
WO 90/13641. Stably transformed eucaryotic cells comprisng a foreign transcribable DNA under the control of a pol III promoter. 1990.
EPO 402226. Transformation vectors for yeast Yarrowia. 1990.
WO 91/00360. Bispecific reagents for AIDS therapy. 1991.
WO 91/04753. Conjugates of antisense oligonucleotides and therapeutic uses thereof. 1991.
U.S. Pat. No. 5,013,556. Liposomes with enhanced circulation time. 1991.
WO 91/06629. Oligonucleotide analogs with novel linkages. 1991.
WO 92/20373. Heteroconjugate antibodies for treatment of HIV infection. 1992.
WO 93/08829. Compositions that mediate killing of HIV-infected cells. 1993.
WO 94/11026. Therapeutic application of chimeric and radiolabeled antibodies to human B lymphocyte restricted differentiation antigen for treatment of B cells. 1994.
WO 96/27011. A method for making heteromultimeric polypeptides. 1996.
U.S. Pat. No. 5,545,807. Production of antibodies from transgenic animals. 1996.
U.S. Pat. No. 5,569,825. Transgenic non-human animals capable of producing heterologous antibodies of various isotypes. 1996.
WO 97/33551. Compositions and methods for the diagnosis, prevention, and treatment of neoplastic cell growth and proliferation. 1997.
U.S. Pat. No. 5,633,425. Transgenic non-human animals capable of producing heterologous antibodies. 1997.
U.S. Pat. No. 5,661,016. Transgenic non-human animals capable of producing heterologous antibodies of various isotypes. 1997.
U.S. Pat. No. 5,625,126. Transgenic non-human animals for producing heterologous antibodies. 1997.
Abravaya, K., J. J. Carrino, S. Muldoon, and H. H. Lee. 1995. Detection of point mutations with a modified ligase chain reaction (Gap-LCR). Nucleic Acids Res. 23:675-82.
Alam, J., and J. L. Cook. 1990. Reporter genes: Application to the study of mammalian gene transcription. Anal Biochem. 188:245-254.
Altschul, S. F., W. Gish, W. Miller, E. W. Myers, et al. 1990. Basic local alignment search tool. J Mol Biol. 215:403-10.
Altschul, S. F., T. L. Madden, A. A. Schaffer, J. Zhang, et al. 1997. Gapped BLAST and PSI-BLAST: a new generation of protein database search programs. Nucleic Acids Res. 25:3389-402.
Aron, D., J. Findling, and J. Tyrrell. 1997. Hypothalamus and pituitary. In Basic & clinical endocrinology. F. Greenspan and G. Strewler, editors. Appleton & Lange, Stamford. 95-156.
Austin, C. P., and C. L. Cepko. 1990. Cellular migration patterns in the developing mouse cerebral cortex. Development. 110:713-732.
Ausubel, F. M., R. Brent, R. E. Kingston, D. D. Moore, et al. 1987. Current protocols in molecular biology. John Wiley & Sons, New York.
Bado, A., S. Levasseur, S. Attoub, S. Kermorgant, J. P. Laigneau, M. N. Bortoluzzi, L. Moizo, T. Lehy, M. Guerre-Millo, Y. Le Marchand-Brustel, and M. J. Lewin. 1998. The stomach is a source of leptin. Nature. 394(6695):790-3.
Barany, F. 1991. Genetic disease detection and DNA amplification using cloned thermostable ligase. Proc Natl Acad Sci USA. 88:189-93.
Bartel, D. P., and J. W. Szostak. 1993. Isolation of new ribozymes from a large pool of random sequences [see comment]. Science. 261:1411-8.
Bartel, P., C. T. Chien, R. Stemglanz, and S. Fields. 1993. Eliminaton of false positives that arise in using the two-hybrid system. Biotechniques. 14:920-4.
Beal, P. A., and P. B. Dervan. 1991. Second structural motif for recognition of DNA by oligonucleotide-directed triple-helix formation. Science. 251:1360-3.
Bechtold, N., and G. Pelletier. 1998. In planta Agrobacterium-mediated transformation of adult Arabidopsis thaliana plants by vacuum infiltration. Methods Mol Biol. 82:259-66.
Beck, B. 2001. KO's and organisation of peptidergic feeding behavior mechanisms. Neurosci Biobehav Rev. 25:143-58.
Becker, D. M., and L. Guarente. 1991. High-efficiency transformation of yeast by electroporation. Methods Enzymol. 194:182-187.
Beggs, J. D. 1978. Transformation of yeast by a replicating hybrid plasmid. Nature. 275:104-109.
Berger, J., J. Hauber, R. Hauber, R. Geiger, et al. 1988. Secreted placental alkaline phosphatase: A powerful new qunatitative indicator of gene expression in eukaryotic cells. Gene. 66:1-10.
Bernardis, L. L., and L. L. Bellinger. 1993. The lateral hypothalamic area revisited: neuroanatomy, body weight regulation, neuroendocrinology and metabolism. Neurosci. Biobehv. Rev. 17: 141-93.
Bernardis, L. L., and L. L. Bellinger. 1996. The lateral hypothalamic area revisited: ingestive behavior. Neurosci. Biobehv. Rev. 20:189-287.
Berns, A., R. Mandag, and H. Te Riele. WO 93/04169. GENE TARGETING IN ANIMAL CELLS USING ISOGENIC DNA CONSTRUCTS. 1993.
Bodine, D. M., K. T. McDonagh, N. E. Seidel, and A. W. Nienhuis. 1991. Survival and retrovirus infection of murine hematopoietic stem cells in vitro: effects of 5-FU and method of infection. Exp. Hematol. 19:206-212.
Boerner, P., R. Lafond, W. Z. Lu, P. Brams, et al. 1991. Production of antigen-specific human monoclonal antibodies from in vitro-primed human splenocytes. J Immunol. 147:86-95.
Boswell, G. A., and R. M. Scribner. U.S. Pat. No. 3,773,919. Polylactide-drug mixtures. 1973.
Bradley. 1987. Teratocarcinomas and Embryonic Stem Cells: A Practical Approach. Oxford University Press, Inc., Oxford. 268 pp.
Bradley, A. 1991. Modifying the mammalian genome by gene targeting. Curr Opin Biotechnol. 2:823-9.
Brauner-Osborne, H., A. A. Jensen, P. O SHeppard, B. Brodin, P. Krogsgaard-Larsen, and P. O'Hara. 2001. Cloning and characterization of a human orphan family C G-protein coupled receptor GPRC5D. Biochimica et Biophysica Acta. 1581:237-248.
Brauner-Osborne, H. and Polv Krogsgaard-Larsen. 2000. Sequence and Expression Pattern of a Novel Human Orphan G-Protein-Coupled REceptor, GPRC5B, a Family C Receptor with a Short Amino-Terminal Domain. Genomics 65:121-128.
Brennan, M., P. F. Davison, and H. Paulus. 1985. Preparation of bispecific antibodies by chemical recombination of monoclonal immunoglobulin GI fragments. Science. 229:81-3.
Brent, R., J. Gyuris, and E. Golemis. WO94/10300. INTERACTION TRAP SYSTEM FOR ISOLATING NOVEL PROTEINS. 1994.
Cancela, J. M. 2001. Specific Ca2+ signaling evoked by cholecystokinin and acetylcholine: the roles of NAADP, cADPR, and IP3. Annu Rev Physiol. 63:99-117.
Capecchi, M. R. 1980. High efficiency transformation by direct microinjection of DNA into cultured mammalian cells. Cell. 22:479.
Capecchi, M. R. 1989. Altering the genome by homologous recombination. Science. 244:1288-92.
Carell, T., E. A. Wintner, and J. Rebek Jr. 1994a. A novel procedure for the synthesis of libraries containing small organic molecules. Angewandte Chemie International Edition. 33:2059-2061.
Carell, T., E. A. Wintner, and J. Rebek Jr. 1994b. A solution phase screening procedure for the isolation of active compounds from a molecular library. Angewandte Chemie International Edition. 33:2061-2064.
Caron, P. C., W. Laird, M. S. Co, N. M. Avdalovic, et al. 1992. Engineered humanized dimeric forms of IgG are more effective antibodies. J Exp Med. 176:1191-5.
Carter, P. 1986. Site-directed mutagenesis. Biochem J. 237:1-7.
Case, M. E., M. Schweizer, S. R. Kushner, and N. H. Giles. 1979. Efficient transformation of Neurospora crassa by utilizing hybrid plasmid DNA. Proc Natl Acad Sci USA. 76:5259-63.
Cech, T. R., F. L. Murphy, and A. J. Zaug. U.S. Pat. No. 5,116,742. RNA ribozyme restriction endoribonucleases and methods. 1992.
Cech, T. R., A. J. Zaug, and M. D. Been. U.S. Pat. No. 4,987,071. RNA ribozyme polymerases, dephosphorylases, restriction endoribonucleases and methods. 1991.
Cepko, C. L., B. E. Roberts, and R. E. Mulligan. 1984. Construction and applications of a highly transmissible murine retrovirus shuttle vector. Cell. 37:1053-1062.
Chalfie, M., Y. tu, G. Euskirchen, W. W. Ward, et al. 1994. Green fluorescent protein as a marker for gene expression. Science. 263:802-805.
Chaney, W. G., D. R. Howard, J. W. Pollard, S. Sallustio, et al. 1986. High-frequency transfection of CHO cells using Polybrene. Somatic Cell Mol. Genet. 12:237.
Chen, C., and H. Okayama. 1988. Calcium phosphate-mediated gene transfer: A highly efficient system for stably transforming cells with plasmid DNA. BioTechniques. 6:632-638.
Chen, S. H., H. D. Shine, J. C. Goodman, R. G. Grossman, et al. 1994. Gene therapy for brain tumors: regression of experimental gliomas by adenovirus-mediated gene transfer in vivo. Proc Natl Acad Sci USA. 91:3054-7.
Cheng, Y. and R. Lotan. 1998. Molecular Cloning and Characterization of a Novel Retinoic Acid-Inducible Gene That Encodes a Putative G Protein-coupled Receptor. Jour. Biol. Chem. 273(52):35008-35015.
Cho, C. Y., E. J. Moran, S. R. Cherry, J. C. Stephans, et al. 1993. An unnatural biopolymer. Science. 261:1303-5.
Clement, K., C. Vaisse, N. Lahlou, S. Cabrol, et al. 1998. A mutation in the human leptin receptor gene causes obesity and pituitary dysfunction. Nature. 392:398-401.
Cohen, A. S., D. L. Smisek, and B. H. Wang. 1996. Emerging technologies for sequencing antisense oligonucleotides: capillary electrophoresis and mass spectrometry. Adv Chromatogr. 36:127-62.
Cohen, J. S. 1989. Oligodeoxynucleotides: Antisense inhibitors of gene expression. CRC Press, Boca Raton, Fla. 255 pp.
Cohen, S. M. N., A. C. Y. Chang, and L. Hsu. 1972. Nonchromosomal antibiotic resistance in bacteria: Genetic transformation of Escherichia coli by R-factor DNA. Proc. Natl. Acad. Sci. USA. 69:2110.
Comuzzie, A. G., and D. B. Allison. 1998. The search for human obesity genes. Science. 280:1374-7.
Cooney, M., G. Czemuszewicz, E. H. Postel, S. J. Flint, et al. 1988. Site-specific oligonucleotide binding represses transcription of the human c-myc gene in vitro. Science. 241:456-9.
Cotton, R. G. 1993. Current methods of mutation detection. Mutat Res. 285:125-44.
Coughlin, S. R. 1994. Expanding horizons for receptors coupled to G proteins: diversity and disease. Curr. Opin. Cell Biol. 6:191-197.
Cronin, M. T., R. V. Fucini, S. M. Kim, R. S. Masino, et al. 1996. Cystic fibrosis mutation detection by hybridization to light-generated DNA probe arrays. Hum Mutat. 7:244-55.
Cull, M. G., J. F. Miller, and P. J. Schatz. 1992. Screening for receptor ligands using large libraries of peptides linked to the C terminus of the lac repressor. Proc Natl Acad Sci USA. 89:1865-9.
Cummings, D. E., J. Q. Purnell, R. S. Frayo, K. Schmidova, B. E. Wisse, and D. S. Weigle. 2001. A Preprandial Rise in Plasma Ghrelin Levels Suggests a Role in Meal Initiation in Humans. Diabetes. 50:1714-1719.
Cwirla, S. E., E. A. Peters, R. W. Barrett, and W. J. Dower. 1990. Peptides on phage: a vast library of peptides for identifying ligands. Proc Natl Acad Sci USA. 87:6378-82.
de Boer, A. G. 1994. Drug absorption enhancement: Concepts, possibilities, limitations and trends. Harwood Academic Publishers, Langhorne, Pa.
de Louvencourt, L., H. Fukuhara, H. Heslot, and M. Wesolowski. 1983. Transformation of Kluyveromyces lactis by killer plasmid DNA. J Bacteriol. 154:737-42.
de Wet, J. R., K. V. Wood, M. DeLuca, D. R. Helinski, et al. 1987. Sturcture and expression in mammalian cells. Mol. Cell Biol. 7:725-737.
Demerec, M., E. A. Adelberg, A. J. Clark, and P. E. Hartman. 1966. A proposal for a uniform nomenclature in bacterial genetics. Genetics. 54:61-76.
Devlin, J. J., L. C. Panganiban, and P. E. Devlin. 1990. Random peptide libraries: a source of specific protein binding molecules. Science. 249:404-6.
DeWitt, S. H., J. S. Kiely, C. J. Stankovic, M. C. Schroeder, et al. 1993. “Diversomers”: an approach to nonpeptide, nonoligomeric chemical diversity. Proc Natl Acad Sci USA. 90:6909-13.
Ebihara, K., Y. Ogawa, H. Masuzaki, M. Shintani, et al. 2001. Transgenic overexpression of leptin rescues insulin resistance and diabetes in a mouse model of lipoatrophic diabetes. Diabetes. 50:1440-8.
Eichelbaum, M., and B. Evert. 1996. Influence of pharmacogenetics on drug disposition and response. Clin Exp Pharmacol Physiol. 23:983-5.
Ellington, A. D., and J. W. Szostak. 1990. In vitro selection of RNA molecules that bind specific ligands. Nature. 346:818-22.
Elmquist, J. K. Elias, C. F. and C. B. Saper. 1999. From lesions to leptin: hypothalamic control of food intake and body weight. Neuron. 22:221-232.
Elroy-Stein, O., and B. Moss. 1990. Cytoplasmic expression system based on constitutive synthesis of bacteriophage T7 RNA polymerase in mammalian cells. Proc. Natl. Acad. Sci. USA. 87:6743-6747.
Eppstein, D. A., E. B. Fraser-Smith, and T. R. Mattews. U.S. Pat. No. 4,522,811. Serial injection of muramyldipeptides and liposomes enhances the anti-infective activity of muramyldipeptides Serial injection of muramyldipeptides and liposomes enhances the anti-infective activity of muramyldipeptides. 1985.
Eppstein, D. A., Y. V. Marsh, M. van der Pas, P. L. Felgner, et al. 1985. Biological activity of liposome-encapsulated murine interferon gamma is mediated by a cell membrane receptor. Proc Natl Acad Sci USA. 82:3688-92.
Escudero, J., and B. Hohn. 1997. Transfer and integration of T-DNA without cell injury in the host plant. Plant Cell. 9:2135-2142.
Evans, R., R. D. Palmiter, and R. L. Brinster. U.S. Pat. No. 4,870,009. Method of obtaining gene product through the generation of transgenic animals. 1989.
Farooqi, I. S., S. A. Jebb, G. Langmack, E. Lawrence, et al. 1999. Effects of recombinant leptin therapy in a child with congenital leptin deficiency. N Engl J Med. 341:879-84.
Fekete, D. M., and C. L. Cepko. 1993. Retroviral infection coupled with tissue transplantation limits gene transfer in the chick embryo. Proc. Natl. Acad. Sci. USA. 90:2350-2354.
Felgner, P. L., T. R. Gadek, M. Holm, R. Roman, et al. 1987. Lipofectin: A highly efficient, lipid-mediated DNA/transfection procedure. Proc. Natl. Acad. Sci. USA. 84:7413-7417.
Felici, F., L. Castagnoli, A. Musacchio, R. Jappelli, et al. 1991. Selection of antibody ligands from a large library of oligopeptides expressed on a multivalent exposition vector. J Mol Biol. 222:301-10.
Fieck, A., D. L. Wyborski, and J. M. Short. 1992. Modifications of the E. coli Lac repressor for expression in eukaryotic cells: effects of nuclear signal sequences on protein activity and nuclear accumulation. Nucleic Acids Res. 20:1785-91.
Finer, J. J., K. R. Finer, and T. Ponappa. 1999. Particle bombardment-mediated transformation. Current Topics in microbiology and immunology. 240:59-80.
Finn, P. J., N. J. Gibson, R. Fallon, A. Hamilton, et al. 1996. Synthesis and properties of DNA-PNA chimeric oligomers. Nucleic Acids Res. 24:3357-63.
Fishwild, D. M., S. L. O'Donnell, T. Bengoechea, D. V. Hudson, et al. 1996. High-avidity human IgG kappa monoclonal antibodies from a novel strain of minilocus transgenic mice [see comments]. Nat Biotechnol. 14:845-51.
Fleer, R., P. Yeh, N. Amellal, I. Maury, et al. 1991. Stable multicopy vectors for high-level secretion of recombinant human serum albumin by Kluyveromyces yeasts. Biotechnology (NY). 9:968-75.
Fodor, S. P., R. P. Rava, X. C. Huang, A. C. Pease, et al. 1993. Multiplexed biochemical assays with biological chips. Nature. 364:555-6.
Fromm, M., L. P. Taylor, and V. Walbot. 1985. Expression of genes transferred into monocot and dicot plant cells by electroporation. Proc. Natl. Acad. Sci. USA. 82:5824-5828.
Fujiki, Y. 2000. Peroxisome biogenesis and peroxisome biogenesis disorders. FEBS Lett. 476:42-6.
Fujita, T., H. Shubiya, T. Ohashi, K. Yamanishi, et al. 1986. Regulation of human interleukin-2 gene: Functional DNA sequences in the 5′ flanking region for the gene expression in activated T lymphocytes. Cell. 46:401-407.
Gabizon, A., R. Shiota, and D. Papahadjopoulos. 1989. Pharmacokinetics and tissue distribution of doxorubicin encapsulated in stable liposomes with long circulation times. J Natl Cancer Inst. 81:1484-8.
Gallagher, S. R. 1992. GUS protocols: Using the GUS gene as a reporter of gene expression. Academic Press, San Diego, Calif.
Gallop, M. A., R. W. Barrett, W. J. Dower, S. P. Fodor, et al. 1994. Applications of combinatorial technologies to drug discovery. 1. Background and peptide combinatorial libraries. J Med Chem. 37:1233-51.
Gasparini, P., A. Bonizzato, M. Dognini, and P. F. Pignatti. 1992. Restriction site generating-polymerase chain reaction (RG-PCR) for the probeless detection of hidden genetic variation: application to the study of some common cystic fibrosis mutations. Mol Cell Probes. 6:1-7.
Gautier, C., F. Morvan, B. Rayner, T. Huynh-Dinh, et al. 1987. Alpha-DNA. IV: Alpha-anomeric and beta-anomeric tetrathymidylates covalently linked to intercalating oxazolopyridocarbazole. Synthesis, physicochemical properties and poly (rA) binding. Nucleic Acids Res. 15:6625-41.
Galvez, T., M. L. Parmentier, C. Joly, B. Malitschek, K. Kaupmann, R. Kuhn, H. Bittiger, W. Froestl, B. Bettler, and J. P. Pin. 1999. Mutagenesis and modeling of the GABA(B) receptor extracellular domain support a Venus flytrap mechanism for ligand binding. J. Biol. Chem. 274:13362-13369.
Gennaro, A. R. 2000. Remington: The science and practice of pharmacy. Lippincott, Williams & Wilkins, Philadelphia, Pa.
Gibbs, R. A., P. N. Nguyen, and C. T. Caskey. 1989. Detection of single DNA base differences by competitive oligonucleotide priming. Nucleic Acids Res. 17:2437-48.
Gietz, R. D., R. A. Woods, P. Manivasakam, and R. H. Schiestl. 1998. Growth and transformation of Saccharomyces cerevisiae. In Cells: A laboratory manual. Vol. I. D. Spector, R. Goldman, and L. Leinwand, editors. Cold Spring Harbor Press, Cold Spring Harbor, N.Y.
Goding, J. W. 1996. Monoclonal antibodies: Principles and Practice. Academic Press, San Diego. 492 pp.
Gorman, C. M., L. F. Moffat, and B. H. Howard. 1982. Recombinant genomes which express chloramphenicol acetyltransferase in mammalian cells. Mol. Cell. Biol. 2:1044-1051.
Graham, F. L., and A. J. van der Eb. 1973. A new technique for the assay of infectivity of human adenovirus 5 DNA. Virology. 52:456-.
Griffin, H. G., and A. M. Griffin. 1993. DNA sequencing. Recent innovations and future trends. Appl Biochem Biotechnol. 38:147-59.
Grompe, M., D. M. Muzny, and C. T. Caskey. 1989. Scanning detection of mutations in human omithine transcarbamoylase by chemical mismatch cleavage. Proc Natl Acad Sci USA. 86:5888-92.
Gruber, M., B. A. Schodin, E. R. Wilson, and D. M. Kranz. 1994. Efficient tumor cell lysis mediated by a bispecific single chain antibody expressed in Escherichia coli. J Immunol. 152:5368-74.
Guan, X. M., H. Yu, and L. H. Van der Ploeg. 1998. Evidence of altered hypothalamic pro-opiomelanocortin/neuropeptide Y mRNA expression in tubby mice. Brain Res Mol Brain Res. 59:273-9.
Guatelli, J. C., K. M. Whitfield, D. Y. Kwoh, K. J. Barringer, et al. 1990. Isothermal, in vitro amplification of nucleic acids by a multienzyme reaction modeled after retroviral replication. Proc Natl Acad Sci USA. 87:1874-8.
Gudas, L. J., M. B. Sporn, and A. B. Roberts. 1994. Cellular biology and biochemistry of the retinoids. In “The Retinoids: Biology, Chemistry and Medicine,” (M. B. Aporn, A. B. Roberts, and D. S. Goodman, Eds.) 2nd. ed. pp. 442-520, Raven Press, New York.
Hanahan, D. 1983. Studies on transformation of Escherichia coli with plasmids. J. Mol. Biol. 166:557-580.
Hansen, G., and M.-D. Chilton. 1999. Lessons in gene transfer to plants by a gifted microbe. Curr. Top. Microbiol. Immunol. 240:21-57.
Hansen, G., and M. S. Wright. 1999. Recent advances in the transformation of plants. Trends Plant Sci. 4:226-231.
Harlow, E., and D. Lane. 1988. Antibodies: A laboratory manual. Cold Spring Harbor Laboratory Press, Cold Spring Harbor. 726 pp.
Harlow, E., and D. Lane. 1999. Using antibodies: A laboratory manual. Cold Spring Harbor Laboratory PRess, Cold Spring Harbor, N.Y.
Haseloff, J., and W. L. Gerlach. 1988. Simple RNA enzymes with new and highly specific endoribonuclease activities. Nature. 334:585-91.
Hayashi, K. 1992. PCR-SSCP: A method for detection of mutations. Genetic and Analytical Techniques Applications. 9:73-79.
Helene, C. 1991. The anti-gene strategy: control of gene expression by triplex-forming-oligonucleotides. Anticancer Drug Des. 6:569-84.
Helene, C., N. T. Thuong, and A. Harel-Bellan. 1992. Control of gene expression by triple helix-forming oligonucleotides. The antigene strategy. Ann NY Acad Sci. 660:27-36.
Heymsfield, S. B., A. S. Greenberg, K. Fujioka, R. M. Dixon, et al. 1999. Recombinant leptin for weight loss in obese and lean adults: a randomized, controlled, dose-escalation trial. Jama. 282:1568-75.
Hill, J. O., and J. C. Peters. 1998. Environmental contributions to the obesity epidemic. Science. 280:1371-4.
Hinnen, A., J. B. Hicks, and G. R. Fink. 1978. Transformation of yeast. Proc. Natl. Acad. Sci. USA. 75:1929-1933.
Hoffman, F. 1996. Laser microbeams for the manipulation of plant cells and subcellular structures. Plant Sci. 113:1-11.
Hofmann, C. and G. Eichele. 1994. Retinoids in development. In “The Retinoids: Biology, Chemistry and Medicine,” (M. B. Sporn, A. B. Roberts, and D. S. GOodman, Eds.), 2nd ed. pp. 443-520, Raven Press, New York.
Hogan, B., Beddington, R., Costantini, F., Lacy, E. 1994. Manipulating the Mouse Embryo: A Laboratory Manual. Cold Spring Harbor Laboratory Press. 500 pp.
Holliger, P., T. Prospero, and G. Winter. 1993. “Diabodies”: small bivalent and bispecific antibody fragments. Proc Natl Acad Sci USA. 90:6444-8.
Hoogenboom, H. R., A. D. Griffiths, K. S. Johnson, D. J. Chiswell, et al. 1991. Multi-subunit proteins on the surface of filamentous phage: methodologies for displaying antibody (Fab) heavy and light chains. Nucleic Acids Res. 19:4133-7.
Houghten, R. A., J. R. Appel, S. E. Blondelle, J. H. Cuervo, et al. 1992. The use of synthetic peptide combinatorial libraries for the identification of bioactive peptides. Biotechniques. 13:412-21.
Hsu, I. C., Q. Yang, M. W. Kahng, and J. F. Xu. 1994. Detection of DNA point mutations with DNA mismatch repair enzymes. Carcinogenesis. 15:1657-62.
Hwang, K. J., K. F. Luk, and P. L. Beaumier. 1980. Hepatic uptake and degradation of unilamellar sphingomyelin/cholesterol liposomes: a kinetic study. Proc Natl Acad Sci USA. 77:4030-4.
Hyrup, B., and P. E. Nielsen. 1996. Peptide nucleic acids (PNA): synthesis, properties and potential applications. Bioorg Med Chem. 4:5-23.
Infante, J. P., and V. A. Huszagh. 2001. Zellweger syndrome knockout mouse models challenge putative peroxisomal beta-oxidation involvement in docosahexaenoic acid (22:6n-3) biosynthesis. Mol Genet Metab. 72:1-7.
Inoue, H., Y. Hayase, A. Imura, S. Iwai, et al. 1987a. Synthesis and hybridization studies on two complementary nona(2′-O-methyl)ribonucleotides. Nucleic Acids Res. 15:6131-48.
Inoue, H., Y. Hayase, S. Iwai, and E. Ohtsuka. 1987b. Sequence-dependent hydrolysis of RNA using modified oligonucleotide splints and RNase H. FEBS Lett. 215:327-30.
Inui, A. 2001. Ghrelin: an orexigenic and somatotropic signal from the stomach. Nat. Rev. Neurosci. 2(8):551-60.
Ishiura, M., S. Hirose, T. Uchida, Y. Hamada, et al. 1982. Phage particle-mediated gene transfer to cultured mammalian cells. Molecular and Cellular Biology. 2:607-616.
Ito, H., Y. Fukuda, K. Murata, and A. Kimura. 1983. Transformation of intact yeast cells treated with alkali cations. J. Bacteriol. 153:163-168.
Iwabuchi, K., B. Li, P. Bartel, and S. Fields. 1993. Use of the two-hybrid system to identify the domain of p53 involved in oligomerization. Oncogene. 8:1693-6.
Jayasena, S. D. 1999. Aptamers: an emerging class of molecules that rival antibodies in diagnostics. Clin Chem. 45:1628-50.
Ji, H. T., M. Grossmann, and I. Ji. 1998. G Prtotein-coupled Receptors. Jour. Biol. Chem. 273(28):17299-17302.
Jones, P. T., P. H. Dear, J. Foote, M. S. Neuberger, et al. 1986. Replacing the complementarity-determining regions in a human antibody with those from a mouse. Nature. 321:522-5.
Karla, S. P. et al., 1999. Interacting appetite-regulating pathways in the hypothalamic regulation of body weight. Endocr. Rev. 20:68-100.
Kaufman, R. J. 1990. Vectors used for expression in mammalian cells. Methods Enzymol. 185:487-511.
Kaufman, R. J., P. Murtha, D. E. Ingolia, C.-Y. Yeung, et al. 1986. Selection and amplification of heterologous genes encoding adenosine deaminase in mammalian cells. Proc. Natl. Acad. Sci. USA. 83:3136-3140.
Kawai, S., and M. Nishizawa. 1984. New procedure for DNA transfection with polycation and dimethyl sulfoxide. Mol. Cell. Biol. 4:1172.
Keen, J., D. Lester, C. Inglehearn, A. Curtis, et al. 1991. Rapid detection of single base mismatches as heteroduplexes on Hydrolink gels. Trends Genet. 7:5.
Kelly, J. M., and M. J. Hynes. 1985. Transformation of Aspergillus niger by the amdS gene of Aspergillus nidulans. Embo J. 4:475-9.
Kersten, S. 2001. Mechanisms of nutritional and hormonal regulation of lipogenesis. EMBO Rep. 2:282-6.
Kostelny, S. A., M. S. Cole, and J. Y. Tso. 1992. Formation of a bispecific antibody by the use of leucine zippers. J Immunol. 148:1547-53.
Koster, H. WO94/16101. DNA SEQUENCING BY MASS SPECTROMETRY. 1994.
Kozal, M. J., N. Shah, N. Shen, R. Yang, et al. 1996. Extensive polymorphisms observed in HIV-1 clade B protease gene using high-density oligonucleotide arrays. Nat Med. 2:753-9.
Kozbor, D., P. Tripputi, J. C. Roder, and C. M. Croce. 1984. A human hybrid myeloma for production of human monoclonal antibodies. J Immunol. 133:3001-5.
Kriegler, M. 1990. Gene transfer and expression: A laboratory manual. Stockton Press, New York. 242 pp.
Kucherlapati, R. S., B. H. Koller, and O. Smithies. WO 91/01140. HOMOLOGOUS RECOMBINATION FOR UNIVERSAL DONOR CELLS AND CHIMERIC MAMMALIAN HOSTS. 1991.
Kwoh, D. Y., G. R. Davis, K. M. Whitfield, H. L. Chappelle, et al. 1989. Transcription-based amplification system and detection of amplified human immunodeficiency virus type I with a bead-based sandwich hybridization format. Proc Natl Acad Sci USA. 86:1173-7.
Ladner, R. C., S. K. Guterman, B. L. Roberts, W. Markland, et al. U.S. Pat. No. 5,223,409. Directed evolution of novel binding proteins. 1993.
Lakso, M., B. Sauer, B. Mosinger, E. J. Lee, et al. 1992. Targeted oncogene activation by site-specific recombination in transgenic mice. Proc Natl Acad Sci USA. 89:6232-6.
Lam, K. S. 1997. Application of combinatorial library methods in cancer research and drug discovery. Anticancer Drug Design. 12:145-167.
Lam, K. S., S. E. Salmon, E. M. Hersh, V. J. Hruby, et al. 1991. General method for rapid synthesis of multicomponent peptide mixtures. Nature. 354:82-84.
Landegren, U., R. Kaiser, J. Sanders, and L. Hood. 1988. A ligase-mediated gene detection technique. Science. 241:1077-80.
Le Mouellic, H., and P. Brullet. WO 90/11354. Process for the specific replacement of a copy of a gene present in the receiver genome via the integration of a gene. 1990.
Leder, P., and T. A. Stewart. U.S. Pat. No. 4,736,866. Transgenic non-human animals. 1988.
Leduc, N., and e. al. 1996. Isolated maize zygotes mimic in vivo embryogenic development and express microinjected genes when cultured in vitro. Dev. Biol. 10:190-203.
Lee, J. S., D. A. Johnson, and A. R. Morgan. 1979. Complexes formed by (pyrimidine)n. (purine)n DNAs on lowering the pH are three-stranded. Nucleic Acids Res. 6:3073-91.
Lee, V. H. L. 1990. Peptide and protein drug delivery. Marcel Dekker, New York, N.Y.
Lefebvre, O., M. P. Chenard, R. Masson, J. Linares, et al. 1996. Gastric mucosa abnormalities and tumorigenesis in mice lacking the pS2 trefoil protein. Science. 274:259-62.
Lemaitre, M., B. Bayard, and B. Lebleu. 1987. Specific antiviral activity of a poly(L-lysine)-conjugated oligodeoxyribonucleotide sequence complementary to vesicular stomatitis virus N protein mRNA initiation site. Proc Natl Acad Sci USA. 84:648-52.
Lemischka, I. R., D. H. Raulet, and R. C. Mulligan. 1986. Developmental potential and dynamic behavior of hematopoietic stem cells. Cell. 45:917-927.
Letsinger, R. L., G. R. Zhang, D. K. Sun, T. Ikeuchi, et al. 1989. Cholesteryl-conjugated oligonucleotides: synthesis, properties, and activity as inhibitors of replication of human immunodeficiency virus in cell culture. Proc Natl Acad Sci USA. 86:6553-6.
Lewin, M. J. and A. Bado. 2001. Gastric Leptin. Microsc. Res. Tech. Rev. 53(5):372-6.
Li, E., T. H. Bestor, and R. Jaenisch. 1992. Targeted mutation of the DNA methyltransferase gene results in embryonic lethality. Cell. 69:915-26.
Linder, M. W., R. A. Prough, and R. Valdes. 1997. Pharmacogenetics: a laboratory tool for optimizing therapeutic efficiency. Clin Chem. 43:254-66.
Littlefield, J. W. 1964. Selection of hybrids from matings of fibroblasts in vitro and their presumed recombinants. Science. 145:709-710.
Lizardi, P. M., C. E. Guerra, H. Lomeli, I. Tussie-Luna, et al. 1988. Exponential amplification of recombinant-RNA hybridization probes. Biotechnology. 6:1197-1202.
Lonberg, N., and D. Huszar. 1995. Human antibodies from transgenic mice. Int Rev Immunol. 13:65-93.
Lonberg, N., L. D. Taylor, F. A. Harding, M. Trounstine, et al. 1994. Antigen-specific human antibodies from mice comprising four distinct genetic modifications [see comments]. Nature. 368:856-9.
Lopata, M. A., D. W. Cleveland, and B. Sollner-Webb. 1984. High-level expression of a chloramphenicol acetyltransferase gene by DEAEdextran-mediated DNA traansfection couled with a dimethylsulfoxide or glycerol shock treatment. Nucleic Acids Research. 12:5707.
Lotan, R. 1996. Retinoids in cancer chemoprevention. FASEB J. 10:1031-1039.
Luckow, V. A. 1991. Cloning and expression of heterologous genes in insect cells with baculovirus vectors. In Recombinant DNA technology and applications. A. Prokop, R. K. Bajpai, and C. Ho, editors. McGraw-Hill, New York. 97-152.
Madura, K., R. J. Dohmen, and A. Varshavsky. 1993. N-recognin/Ubc2 interactions in the N-end rule pathway. J Biol Chem. 268:12046-54.
Maher, L. J. 1992. DNA triple-helix formation: an approach to artificial gene repressors? Bioessays. 14:807-15.
Mandel, M., and A. Higa. 1970. Calcium-dependent bacteriophage DNA infection. J. Mol. biol 53:159-162.
Marasco, W. A., W. A. Haseltine, and S. Y. Chen. 1993. Design, intracellular expression, and activity of a human anti-human immunodeficiency virus type 1 gp120 single-chain antibody. Proc Natl Acad Sci USA. 90:7889-93.
Marks, J. D., A. D. Griffiths, M. Malmqvist, T. P. Clackson, et al. 1992. By-passing immunization: building high affinity human antibodies by chain shuffling. Biotechnology (NY). 10:779-83.
Marks, J. D., H. R. Hoogenboom, T. P. Bonnert, J. McCafferty, et al. 1991. By-passing immunization. Human antibodies from V-gene libraries displayed on phage. J Mol Biol. 222:581-97.
Martin, F. J., and D. Papahadjopoulos. 1982. Irreversible coupling of immunoglobulin fragments to preformed vesicles. An improved method for liposome targeting. J Biol Chem. 257:286-8.
Maxam, A. M., and W. Gilbert. 1977. A new method for sequencing DNA. Proc Natl Acad Sci USA. 74:560-4.
Miller, A. D., and C. Buttimore. 1986. Redesign of retrovirus packaging cell lines to avoid recombination leading to helper virus production. Mol. Cell biol. 6:2895-2902.
Miller, L. K. 1988. Baculoviruses as gene expression vectors. Annu. Rev. Microbiol. 42:177-199.
Milstein, C., and A. C. Cuello. 1983. Hybrid hybridomas and their use in immunohistochemistry. Nature. 305:537-40.
Modrich, P., S.-S. Su, K. G. Au, and R. S. Lahue. U.S. Pat. No. 5,459,039. Methods for mapping genetic mutations. 1995.
Montague, C. T., I. S. Farooqi, J. P. Whitehead, M. A. Soos, et al. 1997. Congenital leptin deficiency is associated with severe early-onset obesity in humans. Nature. 387:903-8.
Morrison, S. L., L. Wims, S. Wallick, L. Tan, et al. 1987. Genetically engineered antibody molecules and their application. Ann NY Acad Sci. 507:187-98.
Mullis, K. B. U.S. Pat. No. 4,683,202. Process for amplifying nucleic acid sequences. 1987.
Mullis, K. B., H. A. Erlish, N. Arnheim, G. T. Horn, et al. U.S. Pat. No. 4,683,195. Process for amplifying, detecting, and/or cloning nucleic acid sequences. 1987.
Munson, P. J., and D. Rodbard. 1980. Ligand: a versatile computerized approach for characterization of ligand-binding systems. Anal Biochem. 107:220-39.
Myers, R. M., Z. Larin, and T. Maniatis. 1985. Detection of single base substitutions by ribonuclease cleavage at mismatches in RNA:DNA duplexes. Science. 230:1242-6.
Nabel, E. G., and G. J. Nabel. U.S. Pat. No. 5,328,470. Treatment of diseases by site-specific instillation of cells or site-specific transformation of cells and kits therefor. 1994.
Naeve, C. W., G. A. Buck, R. L. Niece, R. T. Pon, et al. 1995. Accuracy of automated DNA sequencing: a multi-laboratory comparison of sequencing results. Biotechniques. 19:448-53.
Nakai, K., and P. Horton. 1999. PSORT: a program for detecting sorting signals in proteins and predicting their subcellular localization. Trends Biochem Sci. 24:34-6.
Nakazato, M., N. Murakami, Y. Date, M. Kojima, et al. 2001. A role for ghrelin in the central regulation of feeding. Nature. 409:194-8.
Nakazawa, H., D. English, P. L. Randell, K. Nakazawa, et al. 1994. UV and skin cancer: specific p53 gene mutation in normal skin as a biologically relevant exposure measurement. Proc Natl Acad Sci USA. 91:360-4.
Neumann, E., M. Schaefer-Ridder, Y. Wang, and P. H. Hofschneider. 1982. Gene transfer into mouse lyoma cells by electroporation in high electric fields. EMBO J. 1:841-845.
Norman, R. A., C. Bogardus, and E. Ravussin. 1995. Linkage between obesity and a marker near the tumor necrosis factor-alpha locus in Pima Indians. J Clin Invest. 96:158-62.
O'Gorman, S., D. T. Fox, and G. M. Wahl. 1991. Recombinase-mediated gene activation and site-specific integration in mammalian cells. Science. 251:1351-5.
Okano, H., J. Aruga, T. Nakagawa, C. Shiota, et al. 1991. Myelin basic protein gene and the function of antisense RNA in its repression in myelin-deficient mutant mouse. J Neurochem. 56:560-7.
Oomura Y. 1980. Input-output organization in the hypothalamus relting to food intake behavior. In Handbook of the Hypothalamus: Physiology of the Hypothalamus, ed. P. J. Morgane, J. Panksepp, 2:577-620. New York: Marcel Dekker.
O'Reilly, D. R., L. K. Miller, and V. A. Luckow. 1992. Baculovirus expression vectors. W.H. Freeman and Company, New York.
Orita, M., H. Iwahana, H. Kanazawa, K. Hayashi, et al. 1989. Detection of polymorphisms of human DNA by gel electrophoresis as single-strand conformation polymorphisms. Proc Natl Acad Sci USA. 86:2766-70.
Ou-Lee, T. M., R. Turgeon, and R. Wu. 1986. Uptake and expression of a foreign gene linked to either a plant virus or Drosophila promoter in protoplasts of rice, wheat and sorghum. Proc. Natl. Acad. Sci. USA. 83:6815-6819.
Palmer, T. D., R. A. Hock, W. R. A. osborne, and A. D. Miller. 1987. Efficient retrovirus-mediated transfer and expression of a human adenosine deaminase gene in diploid skin fibroblasts from an adenosie-deficient human. Proc. Natl. Acad. Sci. USA. 84:1055-1059.
Pardridge, W., and P. Schimmel. WO89/10134. Chimeric peptides for neuropeptide delivery through the blood-brain barrier. 1989.
Pear, W., G. Nolan, M. Scott, and D. Baltimore. 1993. Production of high-titer helper-free retroviruses by transient transfection. Proc. Natl. Acad. Sci. USA. 90:8392-8396.
Perry-O'Keefe, H., X. W. Yao, J. M. Coull, M. Fuchs, et al. 1996. Peptide nucleic acid pre-gel hybridization: an alternative to southern hybridization. Proc Natl Acad Sci USA. 93:14670-5.
Perusse, L., and C. Bouchard. 1999. Role of genetic factors in childhood obesity and in susceptibility to dietary variations. Ann Med. 31 Suppl 1: 19-25.
Petersen, K. H., D. K. Jensen, M. Egholm, O. Buchardt, et al. 1976. A PNA-DNA linker synthesis of N-((4,4′-dimethoxytrityloxy)ehtyl)-N-(thymin-1-ylacetyl)glycine. Biorganic and Medicianl Chemistry Letters. 5:1119-1124.
Phillips, M. S., Q. Liu, H. A. Hammond, V. Dugan, et al. 1996. Leptin receptor missense mutation in the fatty Zucker rat. Nat Genet. 13:18-9.
Pi-Sunjer, F. C., and E. NHLBI Obesity Education Initiative Expert Panel on the Identification, and Treatment of Overweight and Obesity in Adults. 1998. Clinical guidelines on the identification, evaluation, and treatment of overweight and obesity in adults. In The evidence report. National Institutes of Health, Bethesda, Md. 263.
Playford, R. J., T. Marchbank, R. A. Goodlad, R. A. Chinery, et al. 1996. Transgenic mice that overexpress the human trefoil peptide pS2 have an increased resistance to intestinal damage. Proc Natl Acad Sci USA. 93:2137-42.
Potter, H. 1988. Electroporation in biology: Methods, applications, and instrumentation. Analytical Biochemistry. 174:361-373.
Potter, H., L. Weir, and P. Leder. 1984. Enhancer-dependent expression of human kappa immunoglobulin genes introduced into mouse pre-B lymphocytes by electroporation. Proc. Natl. Acad. Sci. USA. 81:7161-7165.
Presta, L. G. 1992. Antibody engineering. Curr Opin Biotechnol. 3:394-8.
Prosser, J. 1993. Detecting single-base mutations. Trends Biotechnol. 11:238-46.
Rassoulzadegan, M., B. Binetruy, and F. Cuzin. 1982. High frequency of gene transfer after fusion between bacteria and eukaryotic cells. Nature. 295:257.
Reisfeld, R. A., and S. Sell. 1985. Monoclonal antibodies and cancer therapy: Proceedings of the Roche-UCLA symposium held in Park City, Utah, Jan. 26-Feb. 2, 1985. Alan R. Liss, New York. 609 pp.
Report, A. 1997. Position of the American Dietetic Association: weight management. J Am Diet Assoc. 97:71-4.
Rhodes, C. A., D. A. Pierce, I. J. Mettler, D. Mascarenhas, et al. 1988. Genetically transformed maize plants from protoplasts. Science. 240:204-207.
Riechmann, L., M. Clark, H. Waldmann, and G. Winter. 1988. Reshaping human antibodies for therapy. Nature. 332:323-7.
Robbins, M. J., D. Michalovich, J. Hill, A. R. Calver, A. D. Medhurst, I. Gloger, M. Sims, D. N. Middlemiss, and M. N. Pangalos. 2000. Molecular Cloning and Characterization of Two Novel Retinoic Acid-Inducible Orphan G-Protein-Coupled Receptors (GPRC5B and GPRC5C). Genomics. 67:8-18.
Rose, J. K., L. Buonocore, and M. Whitt. 1991. A new cationic liposome reagent mediating nearly quantitative transfection of animal cells. BioTechniques. 10:520-525.
Rossiter, B. J., and C. T. Caskey. 1990. Molecular scanning methods of mutation detection. J Biol Chem. 265:12753-6.
Saifer, M., R. Somack, and L. D. Williams. U.S. Pat. No. 5,283,317. Intermediates for conjugation of polypeptides with high molecular weight polyalkylene glycols. 1994.
Saiki, R. K., T. L. Bugawan, G. T. Horn, K. B. Mullis, et al. 1986. Analysis of enzymatically amplified beta-globin and HLA-DQ alpha DNA with allele-specific oligonucleotide probes. Nature. 324:163-6.
Saiki, R. K., P. S. Walsh, C. H. Levenson, and H. A. Erlich. 1989. Genetic analysis of amplified DNA with immobilized sequence-specific oligonucleotide probes. Proc Natl Acad Sci USA. 86:6230-4.
Sakurai, T., A. Amemiya, M. Ishii, I. Matsuzaki, et al. 1998a. Orexins and orexin receptors: a family of hypothalamic neuropeptides and G protein-coupled receptors that regulate feeding behavior. Cell. 92:573-85.
Sakurai, T., A. Amemiya, M. Ishii, I. Matsuzaki, et al. 1998b. Orexins and orexin receptors: a family of hypothalamic neuropeptides and G protein-coupled receptors that regulate feeding behavior. Cell. 92:1 page following 696.
Saleeba, J. A., and R. G. Cotton. 1993. Chemical cleavage of mismatch to detect mutations. Methods Enzymol. 217:286-95.
Salton, S. R., S. Hahm, and T. M. Mizuno. 2000. Of mice and MEN: what transgenic models tells us about hypothalamic control of energy balance. Neuron. 25:265-68.
Sambrook, J. 1989. Molecular cloning: a laboratory manual. Cold Spring Harbor Laboratory, Cold Spring Harbor.
Sandri-Goldin, R. M., A. L. Goldin, J. C. Glorioso, and M. Levine. 1981. High-frequency transfer of cloned herpes simjplex virus type I sequences to mammalian cells by protoplast fusion. Mol. Cell. Biol. 1:7453-752.
Sanger, F., S. Nicklen, and A. R. Coulson. 1977. DNA sequencing with chain-terminating inhibitors. Proc Natl Acad Sci USA. 74:5463-7.
Saunders, J. A., B. F. Matthews, and P. D. Miller. 1989. Plant gene transfer using electrofusion and electroporation. In Electroporation and electrofusion in cell biology. E. Neumann, A. E. Sowers, and C. A. Jordan, editors. Plenum Press, New York. 343-354.
Savarese, T. M., C. D. Wang and C. M. Fraser. 1992. Site-directed mutagenesis of the rat ml muscarinic acetylcholine receptor. ROle of conserved cysteines in receptor function. J. Biol. Chem. 266:10807-10812.
Schade, R., C. Staak, C. Hendriksen, M. Erhard, et al. 1996. The production of avian (egg yold) antibodies: IgY. The report and recommendations of ECVAM workshop. Alternatives to Laboratory Animals (ATLA). 24:925-934.
Schaffner, W. 1980. Direct transfer of cloned genes from bacteria to mammalian cells. Proc. Natl. Acad. Sci. USA. 77:2163.
Schoepp, D. D. 2001. Unveiling the functions of presynaptic metabotropic glutamate receptors in the central nervous system. J. Pharmacol. Exp. Ther. 299(1):12-20.
Schook, L. B. 1987. Monoclonal antibody production techniques and applications. Marcel Dekker, Inc., New York. 336 pp.
Schrauwen, P., K. Walder, and E. Ravussin. 1999. Human uncoupling proteins and obesity. Obes Res. 7:97-105.
Scott, J. K., and G. P. Smith. 1990. Searching for peptide ligands with an epitope library. Science. 249:386-90.
Selden, R. F., K. Burke-Howie, M. E. Rowe, H. M. Goodman, et al. 1986. Human growth hormone as a reporter gene in regulation studies employing transient gene expression. Molecular and Cellular Biololgy. 6:3173-3179.
Shalaby, M. R., H. M. Shepard, L. Presta, M. L. Rodrigues, et al. 1992. Development of humanized bispecific antibodies reactive with cytotoxic lymphocytes and tumor cells overexpressing the HER2 protooncogene. J Exp Med. 175:217-25.
Shigekawa, K., and W. J. Dower. 1988. Electroporation of eukaryotes and prokaryotes: A general approach to the introduction of macomolecules into cells. BioTechniques. 6:742-751.
Shillito, R. 1999. Methods of genetic transformations: Electroporation and polyethylene glycol treatment. In Molecular improvement of cereal crop. I. Vasil, editor. Kluwer, Dordrecht, The Netherlands. 9-20.
Shilo, B. Z., and R. A. Weinberg. 1981. DNA sequences homologous to vertebrate oncogenes are conserved in Drosophila melanogaster. Proc Natl Acad Sci USA. 78:6789-92.
Shimkets, R. A., D. G. Lowe, J. T. Tai, P. Sehl, et al. 1999. Gene expression analysis by transcript profiling coupled to a gene database query. Nat Biotechnol. 17:798-803.
Shopes, B. 1992. A genetically engineered human IgG mutant with enhanced cytolytic activity. J Immunol. 148:2918-22.
Simonsen, C. C., and A. D. Levinson. 1983. Isolation and expression of an altered mouse dihydrofolate reductase cDNA. Proc. Natl. Acad. Sci. USA. 80:2495-2499.
Sjarif, D. R., J. K. Ploos van Amstel, M. Duran, F. A. Beemer, et al. 2000. Isolated and contiguous glycerol kinase gene disorders: a review. J Inherit Metab Dis. 23:529-47.
Smulson, M. E., B. Kishor, and H. Konrad. U.S. Pat. No. 5,272,057. Method of detecting a predisposition to cancer by the use of restriction fragment length polymorphism of the gene for human poly (ADP-ribose) polymerase. 1993.
Southern, P. J., and P. Berg. 1982. Transformation of mammalian cells to antibiotic resistanced with a bacterial gene under control of the SV40 early region promoter. J. Mol. Appl. Gen. 1:327-341.
Spiegelman, B. M., and J. S. Flier. 1996. Adipogenesis and obesity: rounding out the big picture. Cell. 87:377-89.
Sreekrishna, K., R. H. Potenz, J. A. Cruze, W. R. McCombie, et al. 1988. High level expression of heterologous proteins in methylotrophic yeast Pichia pastoris. J Basic Microbiol. 28:265-78.
Stein, C. A., and J. S. Cohen. 1988. Oligodeoxynucleotides as inhibitors of gene expression: a review. Cancer Res. 48:2659-68.
Stevenson, G. T., A. Pindar, and C. J. Slade. 1989. A chimeric antibody with dual Fc regions (bisFabFc) prepared by manipulations at the IgG hinge. Anticancer Drug Des. 3:219-30.
Strosberg, A. D. 1997. Structure and function of the beta 3-adrenergic receptor. Annu Rev Pharmacol Toxicol. 37:421-50.
Suresh, M. R., A. C. Cuello, and C. Milstein. 1986. Bispecific monoclonal antibodies from hybrid hybridomas. Methods Enzymol. 121:210-28.
Thomas, K. R., and M. R. Capecchi. 1987. Site-directed mutagenesis by gene targeting in mouse embryo-derived stem cells. Cell. 51:503-12.
Thompson, J., D. Higgins, and T. Gibson. 1994. CLUSTAL W: Improving the sensitivity of progressive multiple sequence alignment through sequence weighting, positions-specific gap penalties and weight matrix choice. Nucl. Ac. Res. 22:4673-4680.
Thompson, J. A., and e. al. 1995. Maize transformation utilizing silicon carbide whiskers: A review. Euphytica. 85:75-80.
Tilburn, J., C. Scazzocchio, G. G. Taylor, J. H. Zabicky-Zissman, et al. 1983. Transformation by integration in Aspergillus nidulans. Gene. 26:205-21.
Toshinai, K., M. S. Mondal, M. Nakazato, Y. Date, N. Murakami, M. Kojima, K. Kangawa, and S. Matsukura. 2001. Upregulation of Ghrelin expression in the stomach upon fastin, insulin-induced hypoglycemia, and leptin administration. Biochem. Biophys. Res. Commun. 281(5):1220-5.
Touraev, A., and e. al. 1997. Plant male germ line transformation. Plant J. 12:949-956.
Traunecker, A., F. Oliveri, and K. Karjalainen. 1991. Myeloma based expression system for production of large mammalian proteins. Trends Biotechnol. 9:109-13.
Trick, H. N., and e. al. 1997. Recent advances in soybean transformation. Plant Tissue Cult. Biotechnol. 3:9-26.
Tschop, M., C. Weyer, P. A. Tataranni, C. Devanarayan, E. Ravussin, and M. L. Heiman. 2001. Circulating Ghrelin LEvels are Decreased in Human Obesity. Diabetes. 50:707-709.
Tuerk, C., and L. Gold. 1990. Systematic evolution of ligands by exponential enrichment: RNA ligands to bacteriophage T4 DNA polymerase. Science. 249:505-10.
Turner, D. L., E. Y. Snyder, and C. L. Cepko. 1990. Lineage-independent determinationh of cell type in the embryonic mouse retina. Neuron. 4:833-845.
Tutt, A., G. T. Stevenson, and M. J. Glennie. 1991. Trispecific F(ab′)3 derivatives that use cooperative signaling via the TCR/CD3 complex and CD2 to activate and redirect resting cytotoxic T cells. J Immunol. 147:60-9.
van der Krol, A. R., J. N. Mol, and A. R. Stuitje. 1988a. Modulation of eukaryotic gene expression by complementary RNA or DNA sequences. Biotechniques. 6:958-76.
van der Krol, A. R., J. N. Mol, and A. R. Stuite. 1988b. Modulation of eukaryotic gene expression by complementary RNA or DNA sequences. Biotechniques. 6:958-76.
Verhoeyen, M., C. Milstein, and G. Winter. 1988. Reshaping human antibodies: grafting an antilysozyme activity. Science. 239:1534-6.
Vitetta, E. S., R. J. Fulton, R. D. May, M. Till, et al. 1987. Redesigning nature's poisons to create anti-tumor reagents. Science. 238:1098-104.
Wagner, T. E., and P. C. Hoppe. U.S. Pat. No. 4,873,191. Genetic transformation of zygotes. 1989.
Weigle, D. S., and J. L. Kuijper. 1996. Obesity genes and the regulation of body fat content. Bioessays. 18:867-74.
Wells, J. A., M. Vasser, and D. B. Powers. 1985. Cassette mutagenesis: an efficient method for generation of multiple mutations at defined sites. Gene. 34:315-23.
Whitt, M. A., L. Buonocore, J. K. Rose, V. Ciccarone, et al. 1990. TransfectACE reagent promotes transient transfection frequencies greater than 90%. Focus. 13:8-12.
Wigler, M., A. Pellicer, S. Silversttein, and R. Axel. 1978. Biochemical transfer of single-copy eucaryotic genes using total cellular DNA as donor. Cell. 14:725.
Wikberg, J. E., R. Muceniece, I. Mandrika, P. Prusis, et al. 2000. New aspects on the melanocortins and their receptors. Pharmacol Res. 42:393-420.
Williams, D. A., I. R. Lemischka, D. G. Nathan, and R. C. Mulligan. 1984. Introduction of a new genetic material into pluripotent haematopoietic stem cells of the mouse. Nature. 310:476-480.
Willie, J. T., R. M. Chemelli, C. M. Sinton and M. Yanagisawa. 2001. To eat or to sleep? Orexin in the regulation of feeding and wakefulness. Annu. Revn Neurosci. 24:429-58.
Wilmut, I., A. E. Schnieke, J. McWhir, A. J. Kind, et al. 1997. Viable offspring derived from fetal and adult mammalian cells. Nature. 385:810-3.
Wolff, E. A., G. J. Schreiber, W. L. Cosand, and H. V. Raff. 1993. Monoclonal antibody homodimers: enhanced antitumor activity in nude mice. Cancer Res. 53:2560-5.
Wong, T. K., and E. Neumann. 1982. Electric field mediated gene transfer. Biochemical and Biophysical Research Communications. 107:584-587.
Wong, W. M., R. Poulsom, and N. A. Wright. 1999. Trefoil peptides. Gut. 44:890-5.
Woods, S. C., R. J. Seely, D. Porte, and M. W. Schwartz. 1998. Signals that regulate food intake and energy homestasis. Science. 280:1378-83.
Wright, N. A., W. Hoffmann, W. R. Otto, M. C. Rio, et al. 1997. Rolling in the clover: trefoil factor family (TFF)-domain peptides, cell migration and cancer. FEBS Lett. 408:121-3.
Wyborski, D. L., L. C. DuCoeur, and J. M. Short. 1996. Parameters affecting the use of the lac repressor system in eukaryotic cells and transgenic animals. Environ Mol Mutagen. 28:447-58.
Wyborski, D. L., and J. M. Short. 1991. Analysis of inducers of the E. coli lac repressor system in mammalian cells and whole animals. Nucleic Acids Res. 19:4647-53.
Yaswen, L., N. Diehl, M. B. Brennan, and U. Hochgeschwender. 1999. Obesity in the mouse model of pro-opiomelanocortin deficiency responds to peripheral melanocortin. Nat Med. 5:1066-70.
Yelton, M. M., J. E. Hamer, and W. E. Timberlake. 1984. Transformation of Aspergillus nidulans by using a trpC plasmid. Proc Natl Acad Sci USA. 81:1470-4.
Zervos, A. S., J. Gyuris, and R. Brent. 1993. Mxil, a protein that specifically interacts with Max to bind Myc-Max recognition sites. Cell. 72:223-32.
Zhou, G., and e. al. 1983. Introduction of exogenous DNA into cotton embryos. Methods Enzymol. 101:433-481.
Zoller, M. J., and M. Smith. 1987. Oligonucleotide-directed mutagenesis: a simple method using two oligonucleotide primers and a single-stranded DNA template. Methods Enzymol. 154:329-50.
Zon, G. 1988. Oligonucleotide analogues as potential chemotherapeutic agents. Pharm Res. 5:539-49.
Zuckermann, R. N., E. J. Martin, D. C. Spellmeyer, G. B. Stauber, et al. 1994. Discovery of nanomolar ligands for 7-transmembrane G-protein-coupled receptors from a diverse N-(substituted)glycine peptoid library. J Med Chem. 37:2678-85.

1. An isolated polypeptide comprising an amino acid sequence having at least 80% sequence identity to the sequence of SEQ ID NO:2.

2-3. (canceled)

4. The polypeptide of claim 1, wherein the amino acid sequence has at least 98% sequence identity to the sequence of SEQ ID NO:2.

5. An isolated polynucleotide encoding the polypeptide of claim 1, or a complement of the polynucleotide.

6. The isolated polynucleotide of claim 5 comprising a polynucleotide sequence having at least 80% sequence identity to the sequence of SEQ ID NO:1, or a complement of the polynucleotide.

7-8. (canceled)

9. An antibody that specifically binds to the polypeptide of claim 1.

10. A method of treating metabolic disorders comprising modulating the activity of a polypeptide having at least 80% sequence identity to the sequence of SEQ ID NO:1 or 3.

11. The method of claim 10, wherein the modulating comprises decreasing the activity of the polypeptide.

12. The method of claim 11, wherein the decreasing activity comprises decreasing the expression of the polypeptide, transforming a cell to express a polynucleotide anti-sense to at least a portion of an endogenous polynucleotide encoding the polypeptide, introducing into a cell an antagonist to the polypeptide, disrupting a gene that encodes the polypeptide or administering to a cell an antibody that selectively binds to the polypeptide.

13-17. (canceled)

18. The method of claim 11, wherein the metabolic disorder is obesity, anorexia, cachexia or diabetes.

19. The method of claim 10, wherein the modulating activity of the polypeptide comprises increasing the activity of the polypeptide.

20. The method of claim 19, wherein the increasing activity comprises increasing the expression of the polypeptide, transforming a cell with a polynucleotide encoding the polypeptide, or administering to a cell an antibody that selectively binds the polypeptide.

21-22. (canceled)

23. The method of claim 10, wherein the modulation comprises controlling the expression with a gene encoding the polypeptide with an exogenous promoter.

24. The method of claim 23, wherein the controlling comprises operably-linking the promoter to an endogenous gene encoding the polypeptide.

25. The method of claim 23, wherein the controlling comprises transforming a cell with a gene encoding the polypeptide operably-linked to a promoter.

26. The method of claim 23, wherein the promoter is an inducible promoter.

27. The method of claim 19, wherein the metabolic disorder is obesity or diabetes.

28. A method of detecting a disorder associated with changes in GPCR-like RAIG1 gene expression comprising: detecting a change in expression or activity of GPCR-like RAIG1 polypeptide.

29. The method of claim 28, wherein the metabolic disorder is associated with an up-regulation of GPCR-like RAIG1 polypeptide activity.

30. The method of claim 29, wherein the metabolic disorder is obesity, anorexia, cachexia or diabetes.

31. The method of claim 28, wherein the metabolic disorder is associated with a down-regulation of GPCR-like RAIG1 polypeptide activity.

32. The method of claim 31, wherein the metabolic disorder is obesity or diabetes.

33. A method for determining whether a compound up-regulates or down-regulates the transcription or translation of a GPCR-like RAIG1 gene, comprising: contacting the compound with a composition comprising a RNA polymerase and the gene and measuring the amount of GPCR-like RAIG1 gene transcription or translation.

34. The method of claim 33, wherein the composition is in a cell.

35-36. (canceled)

37. A vector comprising a polynucleotide encoding the polypeptide of claim 1.

38. A cell, comprising the vector of claim 37.

39. A transgenic non-human animal, comprising a disrupted GPCR-like RAIG1 gene of claim 5.

40. (canceled)

41. A transgenic non-human animal comprising the isolated polynucleotide of claim 6.

42-43. (canceled)

44. A method of screening a sample for a GPCR-like RAIG1 mutation, comprising: comparing a GPCR-like RAIG1 polynucleotide sequence in the sample with SEQ ID NO:1 or SEQ ID NO:3.

45-48. (canceled)

49. An assay for detecting a metabolic disorder, comprising the polypeptide of claim 1.

50. The assay of claim 49, wherein the metabolic disorder is obesity, anorexia, cachexia or diabetes.

51. A kit for treating metabolic disorders, comprising: a container containing the polypeptide of claim 1.

52. The kit of claim 51, wherein the polypeptide comprises a pharmaceutically acceptable carrier.

53. A kit for detecting metabolic disorders, comprising: a container containing the polypeptide of claim 1, the nucleotide of claim 5 or the antibody of claim 9.


SEQUENCE LISTING (20 sequences)

SEQ ID NO: 1 - DNA, 1934 bp, Mus musculus
cccacgcgtctgcccacgcgtcgcgacccacgcgtcctccttgtcccaggacctgcccagtagccaggggttgagcgctctcgcctttcacgcgtgagtcgcaagttctcggttcaccccgagcgccgcagcgcccaggaccaaccagaatgactactcctacaactgcccctagcggttgccgctcagacctggattccaggtaccacagactttgtgacctggcggaaggctggggcatcgcgttggaaacactggctgcagtcggggctgtggctaccgtggcttgcatgtttgcactcgttttcctcatctgcaaagtgcaggactccaacaaaagaaagatgctccccgcccagttcctcttcctcctgggagtgctaggggtctttggcctcaccttcgccttcatcatcaagctggatggggccacaggacccacgcgcttcttcctcttcggagtcctcttcgccatctgcttctcttgcctcctggcccacgctttcaacttgatcaagctggtccgagggaggaagcccctgtcgtggcttgtgatcctcagtctggcggtgggcttcagcctggtccaggacgtcattgccattgaatacctggtcctcaccatgaacaggaccaacgtccatgtcttttctgagctgcactgctcctcggcgcaatgaggatttcgtccatgctcctcatttacgtgctcgtcttgatggtgctgaccttcttcacatccttcttggttttctgtggatccttctctggctggaagagacacgggtttccacatatgcttcacctcgttcctctccattgccatctgggtggcctggatcgttctgctcctgattcccgacattgaccggaaatgggatgataccattctcagcacagccttggtggccaatggctgggttttcctggcattttatatcttgcccgagtttcgacagctcccaaggcaacggagccccactgattacccagttgaagatgctttttgtaaacctcagctcatgaagcagagctatggtgtggagaacagagcctactcccaagaggaaatcacccaaggtcttgagatgggggacacactctatgcaccttattccacccattttcagctacagaatcaccaaaaggatttctctatcccgagggcccaggcccggccagtccgtacaatgactacgaagggcgcaaaggcgacacgtaagtgttgggaagagtgggacaaccagaggcaggtagcaggtccagccaggaatcctgctgatgtgaactgaacctcagggcatcccggggaaacagtacagagaggcttgcaacctgcccagcacaccgccgtcttgcctggggctgctaagcctaacaaactgtcttcaaagagctccagggtttcatttgccccattcctaggacacttctgggaggtgggagtcttggcaactccgggtgagactcttacctctcccgggagtatgagcaagcctccagtcatctgactgctcactgtttggtcatccttggaagccagttcacctaacccacggtgggtcctatgaggagttgctgcacacaatgcaccactcaagattcggaaacgcccagcgaagtatgcgccccggaagaaacctcatcggcgtcctcggacctttggtccaactcgccctcccaaccggccgccccccaggcacctggcacgaaggtcacgtgtctaccctcagtgcactgccccacagtggcctctcggtgcagacaccgatttccaagggccatgtttttatcccaattgcctccaaactcactgccaaccccagaacctctgtgtcctttgccaggagctctttgggacattactggagtagacaaggtctgtttctctctgccaggagaattgggtttgttctcgctataaattcctggcc

SEQ ID NO: 2 - protein, 176 aa, Mus musculus
MTTPTTAPSGCRSDLDSRYHRLCDLAEGWGIALETLAAVGAVATVACMFALVFLICKVQDSNKRKMLPAQFLFLLGVLGVFGLTFAFIIKLDGATGPTRFFLFGVLFAICFSCLLAHAFNLIKLVRGRKPLSWLVILSLAVGFSLVQDVIAIEYLVLTMNRTNVHVFSELHCSSAQ

SEQ ID NO: 3 - DNA, 2302 bp, Homo sapiens
ccaaggtctcccccagcactgaggagctcgcctgctgccctcttgcgcgcgggaagcagcaccaagttcacggccaacgccttggcactagggtccagaatggctacaacagtccctgatggttgccgcaatggcctgaaatccaagtactacagactttgtgataaggctgaagcttggggcatcgtcctagaaacggtggccacagccggggttgtgacctcggtggccttcatgctcactctcccgatcctcgtctgcaaggtgcaggactccaacaggcgaaaaatgctgcctactcagtttctcttcctcctgggtgtgttgggcatctttggcctcaccttcgccttcatcatcggactggacgggagcacagggcccacacgcttcttcctctttgggatcctcttttccatctgcttctcctgcctgctggctcatgctgtcagtctgaccaagctcgtccgggggaggaagcccctttccctgttggtgattctgggtctggccgtgggcttcagcctagtccaggatgttatcgctattgaatatattgtcctgaccatgaataggaccaacgtcaatgtcttttctgagctttccgctcctcgtcgcaatgaagactttgtcctcctgctcacctacgtcctcttcttgatggcgctgaccttcctcatgtcctccttcaccttctgtggttccttcacgggctggaagagacatggggcccacatctacctcacgatgctcctctccattgccatctgggtggcctggatcaccctgctcatgcttcctgactttgaccgcaggtgggatgacaccatcctcagctccgccttggctgccaatggctgggtgttcctgttggcttatgttagtcccgagttttggctgctcacaaagcaacgaaaccccatggattatcctgttgaggatgctttctgtaaacctcaactcgtgaagaagagctatggtgtggagaacagagcctactctcaagaggaaatcactcaaggttttgaagagacaggggacacgctctatgccccctattccacacattttcagctgcagaaccagcctccccaaaaggaattctccatcccacgggcccacgcttggccgagcccttacaaagactatgaagtaaagaaagagggcagctaactctgtcctgaagagtgggacaaatgcagccgggcggcagatctagcgggagctcaaagggatgtgggcgaaatcttgagtcttctgagaaaactgtacaagacactacgggaacagtttgcctccctcccagcctcaaccacaattcttccatgctggggctgatgtgggctagtaagactccagttcttagaggcgctgtagtatttttttttttttgtctcatcctttggatacttcttttaagtgggagtctcaggcaactcaagtttagacccttactctttttgtttgttttttgaaacaggatcttgctctgtcacccaggcttgagtgcagtggtgcgatcacagcccagtgcagcctcgaccacctgtgctcaagcaatcctcccatctccatctcccaaagtgctgggatgacaggcgtgagccacagctcccagcctaggcccttaatcttgctgttattttccatggactaaaggtctggtcatctgagctcacgctggctcacacagctctaggggcctgctcctctaactcacagtgggttttgtgaggctctgtggcccagagcagacctgcatatctgagcaaaaatagcaaaagcctctctcagcccactggcctgaatctacactggaagccaacttgctggcacccccgctccccaacccttcttgcctgggtaggagaggctaaagatcaccctaaatttactcatctctctagtgctgcctcacattgggcctcagcagctccccagcaccaattcacaggtcacccctctcttcttgcactgtccccaaacttgctgtcaattccgagatctaatctccccctacgctctgccaggaattctttcagacctcactagcacaagcccggttgctccttgtcaggagaatttgtagatcattctcacttcaaattcctggggctgatacttctctcatcttgcaccccaacctctgtaaatagatttaccgcatttacggctgcattctgtaagtgggcatggtctcctaatggaggagtgttcattgtataataagttattcacctgagtatgcaataaagatgtggtggccactctttcatggtggtggcagcaaaaaaaaaaaaaa

SEQ ID NO: 4 - protein, 357 aa, Homo sapiens
MATTVPDGCRNGLKSKYYRLCDKAEAWGIVLETVATAGVVTSVAFMLTLPILVCKVQDSNRRKMLPTQFLFLLGVLGIFGLTFAFIIGLDGSTGPTRFFLFGILFSICFSCLLAHAVSLTKLVRGRKPLSLLVILGLAVGFSLVQDVIAIEYIVLTMNRTNVNVFSELSAPRRNEDFVLLLTYVLFLMALTFLMSSFTFCGSFTGWKRHGAHIYLTMLLSIAIWVAWITLLMLPDFDRRWDDTILSSALAANGWVFLLAYVSPEFWLLTKQRNPMDYPVEDAFCKPQLVKKSYGVENRAYSQEEITQGFEETGDTLYAPYSTHFQLQNQPPQKEFSIPRAHAWPSPYKDYEVKKEGS

SEQ ID NO: 5 - DNA, 1619 bp, Homo sapiens
tgcgagggcgggatagctgtccaaggtctcccccagcactgaggagctcgcctgctgccctcttgcgcgcgggaagcagcaccaagttcacggccaacgccttggcactagggtccagaatggctacaacagtccctgatggttgccgcaatggcctgaaatccaagtactacagactttgtgataaggctgaagcttggggcatcgtcctagaaacggtggccacagccggggttgtgacctcggtggccttcatgctcactctcccgatcctcgtctgcaaggtgcaggactccaacaggcgaaaaatgctgcctactcagtttctcttcctcctgggtgtgttgggcatctttggcctcaccttcgccttcatcatcggactggacgggagcacagggcccacacgcttcttcctctttgggatcctcttttccatctgcttctcctgcctgctggctcatgctgtcagtctgaccaagctcgtccgggggaggaagcccctttccctgttggtgattctgggtctggccgtgggcttcagcctagtccaggatgttatcgctattgaatatattgtcctgaccatgaataggaccaacgtcaatgtcttttctgagctttccgctcctcgtcgcaatgaagactttgtcctcctgctcacctacgtcctcttcttgatggcgctgaccttcctcatgtcctccttcaccttctgtggttccttcacgggctggaagagacatggggcccacatctacctcacgatgctcctctccattgccatctgggtggcctggatcaccctgctcatgcttcctgactttgaccgcaggtgggatgacaccatcctcagctccgccttggctgccaatggctgggtgttcctgttggcttatgttagtcccgagttttggctgctcacaaagcaacgaaaccccatggattatcctgttgaggatgctttctgtaaacctcaactcgtgaagaagagctatggtgtggagaacagagcctactctcaagaggaaatcactcaaggttttgaagagacaggggacacgctctatgccccctattccacacattttcagctgcagaaccagcctccccaaaaggaattctccatcccacgggcccacgcttggccgagcccttacaaagactatgaagtaaagaaagagggcagctaactctgtcctgaagagtgggacaaatgcagccgggcggcagatctagcgggagctcaaagggatgtgggcgaaatcttgagtcttctgagaaaactgtacaagacactacgggaacagtttgcctccctcccagcctcaaccacaattcttccatgctggggctgatgtgggctagtaagactccagttcttagaggcgctgtagtatttttttttttttttggctcatccttaggatacttcttttaagtgggagtctcaggcaactcaagtttagacccttactctttttgtttgttttttgaaacaggatcttgctctgtcacccaggcttgagtgcagtggtgcgatcacagcccagtgcagcctcgaccacctgtgctcaagcaatcctcccatctccatctcccaaagtgctgggatgaca

SEQ ID NO: 6 - DNA, 1324 bp, Mus musculus
gaggagagattaaagcacctgaagttgtctcctccttactcatttgcagccacagcttccccaccagcacagcctcagaggcttccggagtagactcggaggaggagaccagacatccgttctcgtgaggtctacctataagtcaccatgtatgaggactgcgtgaagtccacagaagactattacctcttctgtgacaacgaggggccatgggccattgttctggagtccttggcagtgattggcatagtggttaccatattgctactcctggcatttctgttcctcatgcggaaggttcaggactgcagccagtggaacgtgcttcccactcagttcctcttcctgctggctgtgctcgggctcttcggacttacttttgccttcatcatccaactcaaccatcaaactgcccctgttcgctacttcctctttggggttctctttgctatctgcttctcctgcctcctggctcatgcctccaacctggtgaagctggtccggggtagagtctccttctgctggacaacaattctgttcattgctatcggtgtcagcctgttgcagaccatcattgcgatagagtatgtgaccctcatcatgaccagaggcttgatgttcgagcatatgacaccgtatcagctcaatgtggactttgtctgtctcctgatctatgtcctcttcctgatggccctcactttcttcgtctccaaggccaccttctgtggcccatgtgagaactggaaacagcacggaaggctcatatttgctactgtgctggtctctatcattatctgggtggtgtggatctccatgctcttgagaggcaacccccagctccagcgacagccccactgggacgatgcagtcatctgcattggcctggtcaccaacgcttgggtcttcctgctgatctacatcatccctgagctgagcatactctacaggtcatgtaggcaggagtgtcctacacaaggcaacgtctgccaggtccctgtctaccaacgcagcttcaggatggatacccaggaacccaccagagagtgctgatcccagccgggagtatctcatcccatcagctacactaagcccacagcaagatgcaggattgtaaagctactggaaacagcatagagacaacctggaagagtgccctgctccacacagccctaaagagcccaggggagcactggacacactgtcaatgaagcatccttcctgtcccttcctctctgtttccctgccctttccactcttctggacccagcctctgaagactgtcatgtcctgcacaattaaaatcttgttgccaccctaaaaaaaa

SEQ ID NO: 7 - DNA, 1864 bp, Homo sapiens
  modified_base (3)..(3): a, t, c or g
  modified_base (6)..(8): a, t, c or g
  modified_base (10)..(13): a, t, c or g
  modified_base (15)..(15): a, t, c or g
  modified_base (17)..(17): a, t, c or g
  modified_base (19)..(19): a, t, c or g
  modified_base (22)..(23): a, t, c or g
  modified_base (26)..(26): a, t, c or g
  modified_base (28)..(28): a, t, c or g
  modified_base (31)..(33): a, t, c or g
  modified_base (36)..(36): a, t, c or g
  modified_base (39)..(40): a, t, c or g
ggnaannnannnnangntnctnnctngnccnnnacntcnngcacgaggctccctcaccagccggaaagtacgagtcggctcagcctggagggacccaaccagagcctggcctgggagccaggatggccatccacaaagccttggtgatgtgcctgggactgcctctcttcctgttcccaggggcctgggcccagggccatgtcccacccggctgcagccaaggcctcaaccccctgtactacaacctgtgtgaccgctctggggcgtggggcatcgtcctggaggccgtggctggggcgggcattgtcaccacgtttgtgctcaccatcatcctggtggccagcctcccctttgtgcaggacaccaagaaacggagcctgctggggacccaggtattcttccttctggggaccctgggcctcttctgcctcgtgtttgcctgtgtggtgaagcccgacttctccacctgtgcctctcggcgcttcctctttggggttctgttcgccatctgcttctcttgtctggcggctcacgtctttgccctcaacttcctggcccggaagaaccacgggccccggggctgggtgatcttcactgtggctctgctgctgaccctggtagaggtcatcatcaatacagagtggctgatcatcaccctggttcggggcagtggcgagggcggccctcagggcaacagcagcgcaggctgggccgtggcctccccctgtgccatcgccaacatggactttgtcatggcactcatctacgtcatgctgctgctgctgggtgccttcctgggggcctggcccgccctgtgtggccgctacaagcgctggcgtaagcatggggtctttgtgctcctcaccacagccacctccgttgccatatgggtggtgtggatcgtcatgtatacttacggcaacaagcagcacaacagtcccacctgggatgaccccacgctggccatcgccctcgccgccaatgcctgggccttcgtcctcttctacgtcatccccgaggtctcccaggtgaccaagtccagcccagagcaaagctaccagggggacatgtaccccacccggggcgtgggctatgagaccatcctgaaagagcagaagggtcagagcatgttcgtggagaacaaggccttttccatggatgagccggttgcagctaagaggccggtgtcaccatacagcgggtacaatgggcagctgctgaccagtgtgtaccagcccactgagatggccctgatgcacaaagttccgtccgaaggagcttacgacatcatcctcccacgggccaccgccaacagccaggtgatgggcagtgccaactcgaccctgcgggctgaagacatgtactcggcccagagccaccaggcggccacaccgccgaaagacggcaagaactctcaggtctttagaaacccctacgtgtgggactgagtcagcggtggcgaggagaggcgggcggatttggggagggccctgaggacctggccccgggcaagggactctccaggctcctcctccccctggcaggccagcaacatgtgccccagatctggaagggcctccctctctgccagtgtttgggtgggtgtcatgggtgtccccacccactcctcagtgtttgtggagtcgaggagccaaccccagcctcctgccaggatcacctcggcggtcacactccagccaaatagtgttctcggggtggtggctgggcagcgcctatgtttctctggagattcctgcaacctcaagagacttcccaggcgctcaggcctggatcttgctcctctgtgaggaacaagggtgcctaataaatacatttctgctttattaaaaaaaaaaaaaaaaaa

SEQ ID NO: 8 - DNA, 1078 bp, Homo sapiens
  modified_base (3)..(3): a, t, c or g
  modified_base (6)..(8): a, t, c or g
  modified_base (10)..(13): a, t, c or g
  modified_base (15)..(15): a, t, c or g
  modified_base (17)..(17): a, t, c or g
  modified_base (19)..(19): a, t, c or g
  modified_base (22)..(23): a, t, c or g
  modified_base (26)..(26): a, t, c or g
  modified_base (28)..(28): a, t, c or g
  modified_base (30)..(33): a, t, c or g
  modified_base (36)..(36): a, t, c or g
  modified_base (39)..(40): a, t, c or g
ggnaannnannnnangntnctnnctngncnnnnacntcnnatgtacaaggactgcatcgagtccactggagactattttcttctctgtgacgccgaggggccatggggcatcattctggagtccctggccatacttggcatcgtggtcacaattctgctactcttagcatttctcttcctcatgcgaaagatccaagactgcagccagtggaatgtcctccccacccagctcctcttcctcctgagtgtcctggggctcttcggactcgcttttgccttcatcatcgagctcaatcaacaaactgcccccgtacgctactttctctttggggttctctttgctctctgtttctcatgcctcttagctcatgcctccaatctagtgaagctggttcggggttgtgtctccttctcctggacgacaattctgtgcattgctattggttgcagtctgttgcaaatcattattgccactgagtatgtgactctcatcatgaccagaggtatgatgtttgtgaatatgacaccctgccagctcaatgtggactttgttgtactcctggtctatgtcctcttcctgatggccctcacattcttcgtctccaaagccaccttctgtggcccgtgtgagaactggaagcagcatggaaggctcatctttatcactgtgctcttctccatcatcatctgggtggtgtggatctccatgctcctgagaggcaacccgcagttccagcgacagccccagtgggacgacccggtcgtctgcattgctctggtcaccaacgcatgggttttcctgctgctgtacatcgtccctgagctctgcattctctacagatcgtgtagacaggagtgccctttacaaggcaatgcctgccccgtcacagcctaccaacacagcttccaagtggagaaccaggagctctccagagcccgagacagtgatggagctgaggaggatgtagcattaacttcatatggtactcccattcagccgcagactgttgatcccacacaagagtgtttcatcccacaggctaaactaagcccccagcaagatgcaggaggagtataa

SEQ ID NO: 9 - DNA, 3764 bp, Mus musculus
  modified_base (3)..(3): a, t, c or g
  modified_base (6)..(6): a, t, c or g
  modified_base (8)..(9): a, t, c or g
  modified_base (11)..(11): a, t, c or g
  modified_base (15)..(16): a, t, c or g
  modified_base (18)..(18): a, t, c or g
  modified_base (22)..(24): a, t, c or g
  modified_base (27)..(28): a, t, c or g
  modified_base (30)..(30): a, t, c or g
  modified_base (32)..(34): a, t, c or g
  modified_base (36)..(37): a, t, c or g
  modified_base (39)..(39): a, t, c or g
  modified_base (41)..(43): a, t, c or g
ggnaantnntncttnntncacnnntcnntngnnntnngnannncctcctcctcctcctcctcctcctcctcctcctcctcctccttcttcttcttcttcttcttcttcttcttcttcttcgatttactgatttactttgtaagtacactgaagctgtcttcagacacaccagacgaggcatcctatcccattagagatggttgtgagtcaccacgtggttgctgggatttgaactcaggatatttagaagaacagtcagtgctcttaacctctgagccatctctccagtccatccatggttctcttggatactggtctagcttcagtttctatagatgagattgtagaagtttagagagaagccctttccagcctcatcattccccaagtcctttccccaggcccctgtctcttggtgaaccccaggagaggaggagaacttgtcgacctctgacctgcaggccacaagggagatggccaagttaaatattgactgcggtttcttaggtacgagatcaatagaaatatagactccaaagatttaggttgttttggctctggtgtttgaaccagtaccttatacatcacagcataagctcatgaagagtctttctattggtgtgtgtgtgtgtgtgtgtgtgtgtgtgtgtgtgtgtgtgtgtgtgtgtatgtgcgcgtgtgtgtagcactcaccatggtcttgtgtataggtcagaggacaactttacagtcttggttctctccttggacttggttgaccttcgtgccgattttgggaattggactctggttaggcagcacgtggtttattggctgaccttcccatctctctgcctttttgagtcaggctcttgagagtagcccccccctccccctttcccgttgccctatatcactatgtggctcaggctggtggctatcttcctgtttcagtctcccaaatgctgactcgaactgtcatttgcagcaagtcaccttcaggtgacctcaaactcctatgtagtagaaactggccttggagtgtttttctacctgcttctaccacccaagtgctggcattacagaggtgccccgcgccaggcctggcttgttacattttattcttttcttttctttttttgagacagggtttctctgtgcatcccggattgttctggaactcactgtgtagagcagactgacctccaattcactgagatctgcttgcctctcccttaggagtgctgggattaaagttgtgagcccctaccatctggcaagattgtttgttacattttaaaaagcatttaagggtgtggagaaaagtgggtctcagccccttgtaggggttaaacatctttttcacaggggtcacctaagcccagtaaaaagcacagatatgtatattaagattcataacagaagcaaaattacagttatgaggtaataacaaagataattttatggctgggggtcgccatacatgaaaaattgtattaaaactgaactaaagcacttagaaggcctgctccaggccttccctactacctaaaaagcttagttcccccgctgcccaccaacgcaaccaaccgggtaaacctggtccagtgcgttgacattccaggactttttgactacggagcaggcaggtttcagccggactggcctgataatggatgcgctgcggggcccctgcctaagcaggaggaatcagacaccaaaggcaaattccttcatttcaaaattttatctggcgtctctgttcctatcaactgctttgtcaaacttttggagaggtggttaaaagaaaggcgatatgatctctcagtcaggccatgctaacttggctatgcgacagttccaagaagggccttgctgaagcgtctcgacttttccttggtctttactctttagccgcgactgggggacccacaggagagtgagtggctgtctcttctcttttaaacttggcttctccctgctcaccggaggaaggagtgggtgatggagagacaggtgccgccaaggggaaataaagaccaacttctcccgcagaaagtaacaaaggagctgcctggagctgctggctaagtctttgcccttggggccctgtagtgccctcagggaggatgggtgtggctccctccaggagatgacagccagcagcgctcatccatagctgggttcaccgtgtggctcttttcatacgggcgggcgctctgggaccgacctgagcactagtcgagagctacgcctggcagtctagggtgcttcccccgcaaccccgccacctatttcatgagtgactgtgcttctgacgtcagggaggctttcagttcattctctggcacggtgactttattttcacaacagcgagaagtgccgcggaactggattaggcgtgtcctctccctccgccccgcccccgccccttgtccctcggttcaccctctccctccctccggcgacagaagcctttataaccgctttgcgcggccaaggcgggatccttgtcccaggacctgcccagtagccaggggttgagcgctctcgcctttcacgcgtgagtcgcaagttctcggttcaccccgagcgccgcagcgcccaggtaagaggggtttacccaggagcacgctccgggccccagttttaaatccggagagtggggcgtggggagccgtgggaaggagaactgagacagtacagcccgtgatggactacagctggctaggagaaacagatgaaaccgagaaaggaatgtccttaacttctctcacctctgaatgtgttctctgggctaaaacagaggggaaagaccaatcagcaaacagttagaaactgtagcacaggcctgtaattctgcacctgggaggtagatgcagaagaatcaagagttcaaggccagccttgactctactgtgagttccaggtcagcttgggctatataagtctctgtctccagatacaaaacagtagtttaaaataccaatattaaaagagaggtccgtcttgaactgtactaggcccctcccacaggttctatgtagccactggtggaagatgggctatgatgctttcaactcccatctgagtttcaagggtctgtgaatgaatataggggtagagggagaggatgccagctgcttccttcccaaaggagcgaaggccttggaggtcaggaagtgactcacctcaacgcccccccccttctcatgtgcgtgttgttcctccaccccatcccttacttttcctgttgccccagtctctaagaccctgtctctctttaacaagagggggtgaaaagcttgtcttcccggaggagaagagacacaggcaggaggaggctggcagatcacaatgaatggaaagacaggaaacagcagttgtttttttttttttattgttgttgtttttttgtttgtttgtttgttttagacattcaggctggcctcgagcttgtaaccaccattctgcctcagtttgcagagtgttgggataacaggtatcggtctcatggtagaaggctcccaaatggcaaaagcctgcctggaagcaggtaattgaaggaccatcaccgtgcctgcctgccttttctccaggcaagtgtctgaggcttttgttgaagcctggtgaaaggttctctggaaacagtctcttctttgctctttactgaaggagaattctttagccacttcagatacaatggcctgggaaggtggaggggagggtgaggccaacacttcacccttaggggtttttgtactggccatcactgatagagagaa

SEQ ID NO: 10 - protein, 342 aa, Homo sapiens
TTVPDGCRNGLKSKYYRLCDKAEAWGIVLETVATAGVVTSVAFMLTLPILVCKVQDSNRRKMLPTQFLFLLGVLGIFGLTFAFIIGLDGSTGPTRFFLFGILFSICFSCLLAHAVSLTKLVRGRKPLSLLVILGLAVGFSLVQDVIAIEYIVLTMNRTNVNVFSELSAPRRNEDFVLLLTYVLFLMALTFLMSSFTFCGSFTGWKRHGAHIYLTMLLSIAIWVAWITLLMLPDFDRRWDDTILSSALAANGWVFLLAYVSPEFWLLTKQRNPMDYPVEDAFCKPQLVKKSYGVENRAYSQEEITQGFEETGDTLYAPYSTHFQLQNQPPQKEFSIPRAHAWP

SEQ ID NO: 11 - DNA, 25 bp, Artificial Sequence
  Description of Artificial Sequence Synthetic oligonucleotide
ttttttttttttttttttttttttt

SEQ ID NO: 12 - protein, 12 aa, Mus musculus
GFRPCSSFTCSS

SEQ ID NO: 13 - protein, 151 aa, Mus musculus
PSSHPSWFSVDPSLAGRDTGFHICFTSFLSIAIWVAWIVLLLIPDIDRKWDDTILSTALVANGWVFLAFYILPEFRQLPRQRSPTDYPVEDAFCKPQLMKQSYGVENRAYSQEEITQGLEMGDTLYAPYSTHFQLQNHQKDFSIPRAQARP

SEQ ID NO: 14 - protein, 32 aa, Mus musculus
MLLIYVLVLMVLTFFTSFLVFCGSFSGWKRHG

SEQ ID NO: 15 - protein, 32 aa, Homo sapiens
LLLTYVLFLMALTFLMSSFTFCGSFTGWKRHG

SEQ ID NO: 16 - protein, 34 aa, Mus musculus
TLFHPFSATESPKGFLYPEGPGPASPYNDYEGRK

SEQ ID NO: 17 - protein, 40 aa, Homo sapiens
TLYAPYSTHFQLQNQPPQKEFSIPRAHAWPSPYKDYEVKK

SEQ ID NO: 18 - protein, 14 aa, Mus musculus
FLSCTAPRRNEDFV

SEQ ID NO: 19 - protein, 14 aa, Homo sapiens
FSELSAPRRNEDFV

SEQ ID NO: 20 - DNA, 26 bp, Artificial Sequence
  Description of Artificial Sequence Synthetic oligonucleotide
tttttttttttttttttttttttttv